(19) United States
(12) Patent Application Publication
(10) Pub. No.: US 2015/0258197 A1
(43) Pub. Date: Sep. 17, 2015

(54) EUTECTIC ISOMETHEPTENE MUCATE

(71) Applicant: TONIX PHARMA HOLDINGS LIMITED, Hamilton (BM)

(72) Inventors: Seth Lederman, New York, NY (US); Marino Nebuloni, Rho (MI) (IT); Patrizia Colombo, Trezzano sul Naviglio (MI) (IT)

(21) Appl. No.: 14/657,885

(22) Filed: Mar. 13, 2015

Related U.S. Application Data
(60) Provisional application No. 61/953,715, filed on Mar. 14, 2014.

(51) Int. Cl. A61K 47/26; A61K 45/06; A61K 31/131

(52) U.S. Cl. CPC A61K 47/26; A61K 31/131; A61K 45/06

(57) ABSTRACT

The present invention relates to pharmaceutical compositions and methods of manufacturing the same, comprising a eutectic of racemic isometheptene mucate and mannitol or (R)-isometheptene mucate and mannitol.

### RELATED APPLICATION

This application claims priority and benefit from U.S. Provisional Patent Application 61/953,715, filed Mar. 14, 2014, the contents and disclosures of which are incorporated by reference in their entirety.

### BACKGROUND OF THE INVENTION

Racemic isometheptene is an aliphatic amine commercially available as a combination drug product with acetaminophen and dichloralphenazone or acetaminophen and caffeine. It has been used in the treatment of tension-type headache, vascular headache, and migraine headache, either alone or as a combination drug product.

One theory of headache pathogenesis is that cranial vasodilation results in pressure on the pain producing areas surrounding blood vessels. Under this theory, the effect of racemic isometheptene on relieving headache is believed to be due to isometheptene-induced cranial vasoconstriction which reduces the pressure on the pain producing areas surrounding blood vessels. Racemic isometheptene has sympathomimetic effects, and based on pharmacological studies, some of these effects are blocked by α- and β-adrenergic receptor antagonists. This has led some to conclude that racemic isometheptene interacts with α- and β-adrenergic receptors or that its effects are mediated by α- and β-adrenergic receptors indirectly. Racemic isometheptene has also been shown to increase heart rate and diastolic blood pressure, which are properties associated with sympathomimetic agents.

Recent studies have isolated and purified (R)-isometheptene and (S)-isometheptene and demonstrated that (R)-isometheptene has a significant binding affinity for the imidazoline-1 (I₁) receptor (see WO2014/113734, incorporated herein by reference), and binds to I₁ with higher affinity than (S)-isometheptene. Studies have also suggested that (R)-isometheptene may have lower potential adverse effects to the cardiovascular system than (S)-isometheptene.

Development of stable formulations of inert additives of excipients with racemic isometheptene as well as (R)-isometheptene are required.

### SUMMARY OF THE INVENTION

Some embodiments of the invention are:

1. A pharmaceutical composition comprising a eutectic of mannitol and racemic isometheptene mucate.
2. The pharmaceutical composition of embodiment 1, wherein the mannitol is β-mannitol.
3. The pharmaceutical composition of embodiment 1 or 2, wherein the eutectic melts at 142±4° C.
4. The pharmaceutical composition of embodiment 1, wherein the mannitol is δ-mannitol.
5. The pharmaceutical composition of embodiment 1, comprising 60%-90% racemic isometheptene mucate and 40%-10% mannitol by weight.
6. The pharmaceutical composition of embodiment 5, comprising amounts of racemic isometheptene mucate and mannitol selected from: 60%±2% racemic isometheptene mucate and 40%±2% mannitol, 65%±2% racemic isometheptene mucate and 35%±2% mannitol, 70%±2% racemic isometheptene mucate and 30%±2% mannitol, 75%±2% racemic isometheptene mucate and 25%±2% mannitol, 80%±2% racemic isometheptene mucate and 20%±2% mannitol, 85%±2% racemic isometheptene mucate and 15%±2% mannitol, and 90%±2% racemic isometheptene mucate and 10%±2% mannitol by weight.
7. The pharmaceutical composition of embodiment 6, comprising 75%±2% racemic isometheptene mucate and 25%±2% mannitol by weight.
8. The pharmaceutical composition of any one of embodiments 1-7, wherein the racemic isometheptene mucate:mannitol molar ratio is 1.00±0.1:1.00±0.1.
9. The pharmaceutical composition of any one of embodiments 1-8, wherein the racemic isometheptene mucate is micronized racemic isometheptene mucate.
10. The pharmaceutical composition of any one of embodiments 1-9, further comprising one or more excipients.
11. A method of manufacturing a pharmaceutical composition of any one of embodiments 1-10, comprising mixing racemic isometheptene mucate and mannitol or milling racemic isometheptene mucate and mannitol.
12. The method of embodiment 11, comprising milling racemic isometheptene mucate and mannitol.
13. The method of embodiment 12, wherein, the racemic isometheptene mucate and mannitol are milled in a high shear granulator.
14. The method of embodiment 11, comprising mixing racemic isometheptene mucate and mannitol.
15. The method of embodiment 14, wherein the racemic isometheptene mucate and mannitol are mixed via compression.
16. The method of embodiment 15, wherein the racemic isometheptene mucate and mannitol are compressed via roller compaction.
17. A method of manufacturing a pharmaceutical composition of any one of embodiments 1-10, comprising spray drying racemic isometheptene mucate and mannitol.
18. The method of any one of embodiments 11-17, wherein the racemic isometheptene mucate is micronized racemic isometheptene mucate.
19. The method of any one of embodiments 11-18, wherein the pharmaceutical composition further comprises one or more excipients.
20. A pharmaceutical composition comprising a eutectic of mannitol and (R)-isometheptene mucate.
21. The pharmaceutical composition of embodiment 20, wherein the mannitol is β-mannitol.
22. The pharmaceutical composition of embodiment 20 or 21, wherein the eutectic melts at 134±4° C.
23. The pharmaceutical composition of embodiment 20, wherein the mannitol is δ-mannitol.
24. The pharmaceutical composition of embodiment 20 or 23, wherein the eutectic melts at 120±4° C.
25. The pharmaceutical composition of embodiment 20, comprising 60%-90% (R)-isometheptene mucate and 40%-10% mannitol by weight.
26. The pharmaceutical composition of embodiment 25, comprising amounts of (R)-isometheptene mucate and mannitol selected from: 60%±2% (R)-isometheptene mucate and 40%±2% mannitol, 65%±2% (R)-isometheptene mucate and 35%±2% mannitol, 70%±2% (R)-isometheptene mucate and 30%±2% mannitol, 75%±2% (R)-isometheptene mucate and 25%±2% mannitol, 80%±2% (R)-isometheptene mucate and 20%±2% mannitol, 85%±2% (R)-isometheptene mucate and 15%±2% mannitol, and 90%±2% (R)-isometheptene mucate and 10%±2% mannitol by weight.
27. The pharmaceutical composition of embodiment 26, comprising 75%±2% (R)-isometheptene mucate and 25%±2% mannitol by weight.
28. The pharmaceutical composition of any one of embodiments 20-27, wherein the (R)-isometheptene mucate:mannitol molar ratio is 1.00±0.1:1.00±0.1.
29. The pharmaceutical composition of any one of embodiments 20-28, wherein the (R)-isometheptene mucate is micronized (R)-isometheptene mucate.
30. The pharmaceutical composition of any one of embodiments 20-29, further comprising one or more excipients.
31. A method of manufacturing a pharmaceutical composition of any one of embodiments 20-30, comprising mixing (R)-isometheptene mucate and mannitol or milling (R)-isometheptene mucate and mannitol.
32. The method of embodiment 31, comprising milling (R)-isometheptene mucate and mannitol.
33. The method of embodiment 32, wherein, the (R)-isometheptene mucate and mannitol are milled in a high shear granulator.
34. The method of embodiment 31, comprising mixing (R)-isometheptene mucate and mannitol.
35. The method of embodiment 34, wherein the (R)-isometheptene mucate and mannitol are mixed via compression.
36. The method of embodiment 35, wherein the (R)-isometheptene mucate and mannitol are compressed via roller compaction.
37. A method of manufacturing a pharmaceutical composition of any one of embodiments 20-30, comprising spray drying (R)-isometheptene mucate and mannitol.
38. The method of any one of embodiments 31-37, wherein the (R)-isometheptene mucate is micronized (R)-isometheptene mucate.
39. The method of any one of embodiments 31-38, wherein the pharmaceutical composition further comprises one or more excipients.
40. The pharmaceutical composition according to any one of embodiments 1-10 or 20-30 for use as an analgesic.
41. A method of treating or preventing a condition selected from pain, tension-type headache (TTH), allodynia, and fibromyalgia in a patient in need thereof, comprising administering to said patient a therapeutically effective amount of the pharmaceutical composition according to any one of embodiments 1-10 or 20-30.
42. The method of embodiment 41, wherein the condition is pain.
43. The method of embodiment 41, wherein the condition is tension-type headache (TTH).
44. The method of embodiment 41, wherein the condition is allodynia.
45. The method of embodiment 41, wherein the condition is fibromyalgia.
46. The method of any one of embodiments 41-45, wherein the pharmaceutical composition is administered with one or more substances selected from the group consisting of acetaminophen, a non-steroidal anti-inflammatory drug (NSAID), ibuprofen, naprosyn, a cyclooxygenase-2 inhibitor, aspirin, caffeine, dichloralphenazone, a triptan, an antidepressant, a serotonin-norepinephrine reuptake inhibitor (SNRI), and a gabapentinoid.
47. The method of any one of embodiments 41-45, wherein the pharmaceutical composition is administered with one or more additional therapeutics selected from the group consisting of an anti-inflammatory agent, a corticosteroid, a CYP2D6 inhibitor, and a TNF-alpha inhibitor.
48. The method of embodiment 47, wherein the pharmaceutical composition is administered with a CYP2D6 inhibitor.
49. The method of any one of embodiments 41-45, wherein the pharmaceutical composition is administered with one or more opiates.
50. Use of a pharmaceutical composition according to any one of embodiments 1-10 or 20-30 for the manufacture of a medicament for use as an analgesic.
51. Use of a pharmaceutical composition according to any one of embodiments 1-10 or 20-30 for the manufacture of a medicament for treating a condition selected from pain, tension-type headache (TTH), allodynia, and fibromyalgia.
52. The use of embodiment 51, wherein the condition is pain.
53. The use of embodiment 51, wherein the condition is tension-type headache (TTH).
54. The use of embodiment 51, wherein the condition is allodynia.
55. The use of embodiment 51, wherein the condition is fibromyalgia.
56. The use of any one of embodiments 50-55, wherein the pharmaceutical composition is administered with one or more substances selected from the group consisting of acetaminophen, a non-steroidal anti-inflammatory drug (NSAID), ibuprofen, naprosyn, a cyclooxygenase-2 inhibitor, aspirin, caffeine, dichloralphenazone, a triptan, an antidepressant, a serotonin-norepinephrine reuptake inhibitor (SNRI), and a gabapentinoid.
57. The use of any one of embodiments 50-55, wherein the pharmaceutical composition is administered with one or more additional therapeutics selected from the group consisting of an anti-inflammatory agent, a corticosteroid, a CYP2D6 inhibitor, and a TNF-alpha inhibitor.
58. The use of embodiment 57, wherein the pharmaceutical composition is administered with a CYP2D6 inhibitor.
59. The use of any one of embodiments 50-55, wherein the pharmaceutical composition is administered with one or more opiates.

### BRIEF DESCRIPTION OF THE DRAWINGS

FIG. 1: DSC heating curve of racemic isometheptene mucate.

FIG. 2: DSC heating curve of racemic isometheptene mucate+mannitol SD 1:1.

FIG. 3: DSC heating curves of racemic isometheptene mucate+mannitol SD 1:1, 1:3, and 3:1.

FIG. 4: X-ray powder diffraction (XRPD) of racemic isometheptene mucate+mannitol SD 1:3 and 3:1, racemic isometheptene mucate, and mannitol beta.

FIG. 5: XRPD of racemic isometheptene mucate+mannitol SD (1:3) compared to racemic isometheptene mucate alone and different mannitols (α, β, and δ).

FIG. 6: DSC heating curve of racemic isometheptene mucate+magnesium stearate 1:1.

FIG. 7: DSC heating curve of racemic isometheptene mucate+Ac-Di-Sol 1:1.

FIG. 8: DSC heating curve of racemic isometheptene mucate+plasdone 1:1.

FIG. 9: DSC heating curve of racemic isometheptene mucate+colloidal silica 1:1.

FIG. 10: DSC heating curve of racemic isometheptene mucate+crospovidone stearate 1:1.

FIG. 11: DSC heating curve of racemic isometheptene mucate+stearic acid 1:1.

FIG. 12: DSC heating curve of racemic isometheptene mucate+isomalt 1:1.

FIG. 13: DSC heating curve of racemic isometheptene mucate+mannitol 1:1.

FIG. 14: DSC heating curve of racemic isometheptene mucate+opadry II clear 1:1.

FIG. 15: DSC heating curve of racemic isometheptene mucate+povidone 1:1.

FIG. 16: DSC heating curve of racemic isometheptene mucate+mannitol 1:1.

FIG. 17: DSC heating curve of racemic isometheptene mucate+extra granules isomalt 1:1.

FIG. 18: DSC heating curves of racemic isometheptene mucate and (R)-isometheptene mucate.

FIG. 19: XRPD of racemic isometheptene mucate and (R)-isometheptene mucate.

FIG. 20: DSC heating curves of racemic isometheptene mucate and (R)-isometheptene mucate (90:10, 75:25, 62.5:37.5, 55:45, 50:50, 30:70, and 25:75 racemic isometheptene mucate:(R)-isometheptene mucate).

FIG. 21: XRPD of mixtures of racemic isometheptene mucate and (R)-isometheptene mucate.

FIG. 22: Phase diagram of mixtures of racemic isometheptene mucate and (R)-isometheptene mucate.

FIGS. 23 and 23A: DSC heating curves of racemic isometheptene mucate+mannitol SD 1:1, 1:3, and 3:1 and 60:40, 40:60, and 90:10 racemic isometheptene mucate:mannitol.

FIG. 24: XRPD of mixtures of racemic isometheptene mucate and mannitol (different region)

FIG. 25: Phase diagram of mixtures of racemic isometheptene mucate and β mannitol.

FIG. 26: DSC heating curves of racemic isometheptene mucate+mannitol SD 1:1 and (R)-isometheptene mucate+mannitol SD 1:1.

FIG. 27: DSC heating curves of racemic isometheptene mucate+magnesium stearate 1:1 and (R)-isometheptene mucate+magnesium stearate 1:1.

FIG. 28: DSC heating curves of racemic isometheptene mucate+plasdone 1:1 and (R)-isometheptene mucate+plasdone 1:1.

FIG. 29: DSC heating curves of racemic isometheptene mucate+colloidal silica 1:1 and (R)-isometheptene mucate+colloidal silica 1:1.

FIG. 30: DSC heating curves of racemic isometheptene mucate+crospovidone 1:1 and (R)-isometheptene mucate+crospovidone 1:1.

FIG. 31: DSC heating curves of racemic isometheptene mucate+isomalt 1:1 and (R)-isometheptene mucate+isomalt 1:1.

FIG. 32: DSC heating curves of (R)-isometheptene mucate and mannitol (90:10, 75:25, 60:40, 50:50, 40:60, and 25:75 (R)-isometheptene mucate:mannitol).

FIG. 33: XRPD of mixtures of (R)-isometheptene mucate and mannitol.

FIG. 34: Phase diagram of mixtures of (R)-isometheptene mucate and 13 mannitol.

FIG. 35: DSC heating curves of mechanical granulations of 75:25 (R)-isometheptene mucate:mannitol and wet granulations of 75:25 (R)-isometheptene mucate:mannitol.

FIG. 36: DSC heating curve of a fast evaporation test with 75:25 (R)-isometheptene mucate:mannitol in 1:1 water:ethanol.

FIG. 37: DSC heating curve of a spray dry test with 75:25 (R)-isometheptene mucate:mannitol. Spray dry yields a composition with δ-mannitol.

FIG. 38: XRPD of a spray dry test with 75:25 (R)-isometheptene mucate:mannitol.

FIG. 39: XRPD of mixtures made using spray dry or mechanical granulations with 75:25 (R)-isometheptene mucate:mannitol.

FIG. 40: DSC heating curve of (R)-isometheptene maleate.

FIG. 41: XRPD of (R)-isometheptene maleate.

FIG. 42: DSC heating curves of mechanical granulations of 75:25 (R)-isometheptene maleate:mannitol and wet granulations of 75:25 (R)-isometheptene maleate:mannitol.

FIG. 43: XRPD of 75:25 (R)-isometheptene maleate:mannitol, β-mannitol, and (R)-isometheptene maleate.

FIG. 44: DSC heating curve of (R)-isometheptene malate.

FIG. 45: XRPD of (R)-isometheptene malate.

FIG. 46: DSC heating curves of mechanical granulations of 75:25 (R)-isometheptene malate:mannitol and wet granulations of 75:25 (R)-isometheptene malate:mannitol.

FIG. 47: XRPD of 75:25 (R)-isometheptene malate:mannitol, β-mannitol, and (R)-isometheptene malate.

FIG. 48: DSC heating curve of (R)-isometheptene tartrate.

FIG. 49: XRPD of (R)-isometheptene tartrate.

FIG. 50: DSC heating curves of mechanical granulations of 75:25 (R)-isometheptene tartrate:mannitol and wet granulations of 75:25 (R)-isometheptene tartrate:mannitol.

FIG. 51: XRPD of 75:25 (R)-isometheptene tartrate:mannitol, β-mannitol, and (R)-isometheptene tartrate.

FIG. 52: Data from the evaluation of (R)-isometheptene mucate and (S)-isometheptene mucate for analgesic activity using the Formalin Test, late phase (licking score) in a mouse.

FIG. 53: Data from the evaluation of (R)-isometheptene mucate and (S)-isometheptene mucate for analgesic activity using the Tail-flick Test in a mouse.

FIG. 54: Data from the evaluation of (R)-isometheptene mucate and (S)-isometheptene mucate for analgesic activity using the Hot Plate Test in a mouse.

FIG. 55: Data from tactile sensory testing in STA rats using von Frey monofilaments for (R)-isometheptene mucate and (S)-isometheptene mucate.

FIG. 56: DSC curves of Formulation 1 at t=0 and after storage at 50° C. for 1 month.

FIG. 57: XRPD of Formulation 1 at t=0 and after storage at 50° C. for 1 month.

FIG. 58: DSC curves of Formulation 2 at t=0 and after storage at 50° C. for 1 month.

FIG. 59: XRPD of Formulation 2 at t=0 and after storage at 50° C. for 1 month.

### DETAILED DESCRIPTION OF THE INVENTION

Unless otherwise defined herein, scientific and technical terms used in this application shall have the meanings that are commonly understood by those of ordinary skill in the art. Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, chemistry described herein, are those well known and commonly used in the art.

The methods and techniques of the present invention are generally performed, unless otherwise indicated, according to conventional methods well known in the art and as described in various general and more specific references that are cited and discussed throughout this specification.

Chemistry terms used herein are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

A “patient”, “subject”, or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” or “treatment” of a condition or patient refers to taking steps to obtain beneficial or desired results, including clinical results. Beneficial or desired clinical results include, but are not limited to, alleviation or amelioration of one or more symptoms associated with a disease or condition as described herein.

“Administering” or “administration of” a substance, a compound or an agent to a subject can be carried out using one of a variety of methods known to those skilled in the art. For example, a compound or an agent can be administered sublingually or intranasally, by inhalation into the lung or rectally. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

### Compounds

The compounds useful in embodiments of the present invention include racemic isometheptene mucate (IMH) and (R)-isometheptene mucate [(R)-IMH]. In some embodiments, the compounds are micronized. In alternative embodiments, the compounds are not micronized. In some embodiments, the compounds may be present in one or more crystal isoforms.

As used herein, “racemic isometheptene mucate” refers to the pharmaceutically acceptable racemic (RS)-isometheptene mucate salt of racemic isometheptene.

As used herein, “(R)-isometheptene mucate” refers to the pharmaceutically acceptable (R)-isometheptene mucate salt of racemic (R)-isometheptene. The structure of (R)-isometheptene mucate is:

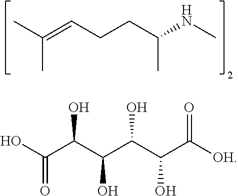

As used herein, “(S)-isometheptene mucate” refers to the pharmaceutically acceptable (S)-isometheptene mucate salt of racemic (S)-isometheptene. The structure of (S)-isometheptene mucate is:

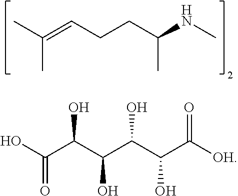

### Eutectic Compositions

In solid drug product formulation, the knowledge of possible interactions between the drug substance and the excipients is a crucial point for the prediction of chemical and physical stability.

Very often, the excipients can modify the biological activity and chemical stability of the API because the dissolution or chemical structures are changed. In some cases, the excipient can improve the chemical stability profile over time and avoid undesirable physical behavior of the final dosage form.

A eutectic is a mixture of chemical compounds or elements that has a single chemical composition that melts at a lower temperature than any other composition made up of the same ingredients. A composition comprising a eutectic is known as the eutectic composition. The melting temperature of a eutectic is known as the eutectic temperature. To define a eutectic, a binary phase diagram should be built by analyzing different compounds ratios.

The effect of a eutectic on tablet properties shows that compaction provides the intimate contact and mutual solubility sufficient for eutectic formation. Eutectic compositions often have higher stability and/or dissolution rates than their non-eutectic counterparts. Because eutectics enhance dissolution, they can be employed to increase permeability in solid dispersions and dispersion systems. However, in the development of certain tableted dosage forms, undesired eutectic formation (during manufacturing operation such as wet granulation), can lead to unwanted changes in physical or chemical characteristics of the tablet, such as low eutectic melting temperature, sticking, unpredictable hardness, instability or difficulties in accelerated assessment of stability.

Mannitol is an excipient commonly used in solid drug products. Mannitol is a 6-carbon sugar alcohol. Sugar alcohols are hydrogenated carbohydrates whose carbonyl group has been reduced to a primary or secondary hydroxyl group. Other 6-carbon sugar alcohols include sorbitol, inositol, galactitol, fucitol, and iditol.

Although mannitol can be included in pharmaceutical compositions, it is typically because it provides qualitative benefits such as sweet taste or a cooling effect in the mouth, but is physically inert. Thus, it was surprising to discover that mannitol formed a eutectic composition with racemic or (R)-isometheptene mucate. Without wishing to be bound by theory, it is possible that the two co-penetrating crystal lattices of mannitol and isometheptene provide protection of the isometheptene from other chemical interactions. Interestingly, mannitol does not form a eutectic composition with (R)-isometheptene maleate, (R)-isometheptene malate, or (R)-isometheptene tartrate. Without wishing to be bound by theory, it is possible that the extra hydroxyl groups on mucate compared to maleate, malate, or tartrate may interact more strongly with mannitol.

In some embodiments, the invention provides a pharmaceutical composition comprising a eutectic mixture of mannitol and an active pharmaceutical ingredient. In certain embodiments, the active pharmaceutical ingredient is racemic isometheptene mucate or (R)-isometheptene mucate. Without wishing to be bound by theory, in a eutectic composition with mannitol, racemic or (R)-isometheptene mucate can be solubilized by binding to the mannitol, possibly improving rapidity of onset and bioavailability.

In some embodiments, the invention provides a pharmaceutical composition comprising a eutectic mixture of mannitol and racemic isometheptene mucate. In certain embodiments (for example, when the composition comprises a β mannitol eutectic), the eutectic has a melting temperature of 142.0±4° C. In certain embodiments, a melting temperature of the eutectic is approximately 132.0° C., 133.0° C., 134.0° C., 135.0° C., 136.0° C., 137.0° C., 138.0° C., 139.0° C., 140.0° C., 141.0° C., 142.0° C., 143.0° C., 144.0° C., 145.0° C., 146.0° C., 147.0° C., 148.0° C., 149.0° C., 150.0° C., 151.0° C., or 152.0° C. In particular embodiments, the melting temperature of the eutectic is the temperature at which melting begins. In alternative embodiments, the melting temperature of the eutectic is the temperature at which maximum melting is observed. In certain embodiments, the composition comprises greater than about 5% racemic isometheptene mucate and less than about 95% mannitol by weight. In certain embodiments, the composition comprises about 1%-5% racemic isometheptene mucate and about 99%-95% mannitol by weight. In certain embodiments, the composition comprises about 5%40% racemic isometheptene mucate and about 95%-90% mannitol by weight. In certain embodiments, the composition comprises about 10%-20% racemic isometheptene mucate and about 90%-80% mannitol by weight. In certain embodiments, the composition comprises about 10%-90% racemic isometheptene mucate and about 90%-10% mannitol by weight, for example, about 60%-90% racemic isometheptene mucate and about 40%-10% mannitol or about 70%-80% racemic isometheptene mucate and about 30%-20% mannitol by weight. Exemplary compositions comprise 25%±2% racemic isometheptene mucate and 75%±2% mannitol, 35%±2% racemic isometheptene mucate and 65%±2% mannitol, 40%±2% racemic isometheptene mucate and 60%±2% mannitol, 50%±2% racemic isometheptene mucate and 50%±2% mannitol, 60%±2% racemic isometheptene mucate and 40%±2% mannitol, 65%±2% racemic isometheptene mucate and 35%±2% mannitol, 70%±2% racemic isometheptene mucate and 30%±2% mannitol, 75%±2% racemic isometheptene mucate and 25%±2% mannitol, 80%±2% racemic isometheptene mucate and 20%±2% mannitol, 85%±2% racemic isometheptene mucate and 15%±2% mannitol, and 90%±2% racemic isometheptene mucate and 10%±2% mannitol by weight. In certain embodiments, a composition comprises 75%±10% racemic isometheptene mucate and 25%±10% mannitol by weight. In certain embodiments, a composition comprises 75%±2% racemic isometheptene mucate and 25%±2% mannitol by weight. In certain embodiments, a composition comprises 75% racemic isometheptene mucate and 25% mannitol by weight. In certain embodiments, the composition comprises racemic isometheptene mucate and mannitol in a racemic isometheptene mucate:mannitol molar ratio of 0.50±0.1:1.0±0.1 to 1.50±0.1:1.0±0.1. In certain embodiments, the molar ratio is about 0.8:1.0 to 1.2:1.0. In particular embodiments, the molar ratio is 0.6±0.1:1.0±0.1, 0.7±0.1:1.0±0.1, 0.8±0.1:1.0±0.1, 0.9±0.1:1.0±0.1, 1.0±0.1:1.0±0.1, 1.1±0.1:1.0±0.1, 1.2±0.1:1.0±0.1, 1.3±0.1:1.0±0.1, 1.4±0.1:1.0±0.1, or 1.5±0.1:1.0±0.1. In certain embodiments, the molar ratio is 0.6±0.5:1.0±0.5, 0.7±0.5:1.0±0.5, 0.8±0.5:1.0±0.5, 0.9±0.5:1.0±0.5, 1.0±0.5:1.0±0.5, 1.1±0.5:1.0±0.5, 1.2±0.5:1.0±0.5, 1.3±0.5:1.0±0.5, 1.4±0.5:1.0±0.5, or 1.5±0.5:1.0±0.5. In certain embodiments the molar ratio is 1.0±0.1:1.0±0.1. In certain embodiments the molar ratio is 1.0±0.5:1.0±0.5.

In some embodiments, the invention provides a pharmaceutical composition comprising a eutectic mixture of mannitol and (R)-isometheptene mucate. In certain embodiments, the composition has a melting temperature of 134±4° C. In certain embodiments, a melting temperature of the composition is approximately 124° C., approximately 125° C., approximately 126° C., approximately 127° C., approximately 128° C., approximately 129° C., approximately 130° C., approximately 131° C., approximately 132° C., approximately 133° C., approximately 134° C., approximately 135° C., approximately 136° C., approximately 137° C., approximately 138° C., approximately 139° C., approximately 140° C., approximately 141° C., approximately 142° C., approximately 143° C., or approximately 144° C. In certain embodiments (for example, when the composition comprises a δ mannitol eutectic), the eutectic has a melting temperature of 120±4° C. In certain embodiments (for example, when the composition comprises a δ mannitol eutectic), a melting temperature of the eutectic is approximately 112° C., approximately 113° C., approximately 114° C., approximately 115° C., approximately 116° C., approximately 117° C., approximately 118° C., approximately 119° C., approximately 120° C., approximately 121° C., approximately 122° C., approximately 123° C., approximately 124° C., approximately 125° C., approximately 126° C., approximately 127° C., approximately 128° C., approximately 129° C., approximately 130° C., approximately 131° C., approximately 132° C., approximately 133° C., or approximately 134° C. In particular embodiments, the melting temperature of the eutectic is the temperature at which melting begins. In alternative embodiments, the melting temperature of the eutectic is the temperature at which maximum melting is observed. In certain embodiments, the composition comprises greater than approximately 5% (R)-isometheptene mucate and less than approximately 95% mannitol by weight. In certain embodiments, the composition comprises 1%-approximately 5% (R)-isometheptene mucate and approximately 99%-95% mannitol by weight. In certain embodiments, the composition comprises approximately 5%40% (R)-isometheptene mucate and approximately 95%-90% mannitol by weight. In certain embodiments, the composition comprises approximately 10%-20% (R)-isometheptene mucate and approximately 90%-80% mannitol by weight. In certain embodiments, the composition comprises approximately 10%-90% (R)-isometheptene mucate and approximately 90%-10% mannitol by weight, for example, approximately 60%-90% (R)-isometheptene mucate and approximately 40%-10% mannitol or approximately 70%-80% (R)-isometheptene mucate and approximately 30%-20% mannitol by weight. Exemplary compositions comprise 25%±2% (R)-isometheptene mucate and 75%±2% mannitol, 35%±2% (R)-isometheptene mucate and 65%±2% mannitol, 40%±2% (R)-isometheptene mucate and 60%±2% mannitol, 50%±2% (R)-isometheptene mucate and 50%±2% mannitol, 60%±2% (R)-isometheptene mucate and 40%±2% mannitol, 65%±2% (R)-isometheptene mucate and 35%±2% mannitol, 70%±2% (R)-isometheptene mucate and 30%±2% mannitol, 75%±2% (R)-isometheptene mucate and 25%±2% mannitol, 80%±2% (R)-isometheptene mucate and 20%±2% mannitol, 85%±2% (R)-isometheptene mucate and 15%±2% mannitol, and 90%±2% (R)-isometheptene mucate and 10%±2% mannitol by weight. In certain embodiments, a composition comprises 75%±10% (R)-isometheptene mucate and 25%±10% mannitol by weight. In certain embodiments, a composition comprises 75%±2% (R)-isometheptene mucate and 25%±2% mannitol by weight. In certain embodiments, a composition comprises 75% (R)-isometheptene mucate and 25% mannitol by weight. In certain embodiments, the composition comprises (R)-isometheptene mucate and mannitol in a (R)-isometheptene mucate:mannitol molar ratio of 0.50±0.1:1.0±0.1 to 1.50±0.1:1.0±0.1. In certain embodiments, the molar ratio is about 0.8:1.0 to 1.2:1.0. In particular embodiments, the molar ratio is 0.6±0.1:1.0±0.1, 0.7±0.1:1.0±0.1, 0.8±0.1:1.0±0.1, 0.9±0.1:1.0±0.1, 1.0±0.1:1.0±0.1, 1.1±0.1:1.0±0.1, 1.2±0.1:1.0±0.1, 1.3±0.1:1.0±0.1, 1.4±0.1:1.0±0.1, or 1.5±0.1:1.0±0.1. In certain embodiments, the molar ratio is 0.6±0.5:1.0±0.5, 0.7±0.5:1.0±0.5, 0.8±0.5:1.0±0.5, 0.9±0.5:1.0±0.5, 1.0±0.5:1.0±0.5, 1.1±0.5:1.0±0.5, 1.2±0.5:1.0±0.5, 1.3±0.5:1.0±0.5, 1.4±0.5:1.0±0.5, or 1.5±0.5:1.0±0.5. In certain embodiments the molar ratio is 1.0±0.1:1.0±0.1. In certain embodiments the molar ratio is 1.0±0.5:1.0±0.5.

In certain embodiments, the invention provides a tablet containing a eutectic comprising racemic isometheptene mucate or (R)-isometheptene mucate and mannitol. In some embodiments, the invention provides a pharmaceutical composition comprising racemic isometheptene mucate or (R)-isometheptene mucate and mannitol, wherein the composition may have an increased stability in tablet form as compared to the same tablet without mannitol, e.g., to a tablet comprising isomalt but not mannitol.

In some embodiments, the invention provides a pharmaceutical composition comprising racemic isometheptene mucate and mannitol or (R)-isometheptene mucate and mannitol, wherein the composition has an increased dissolution rate of a stable tablet compared to racemic isometheptene mucate or (R)-isometheptene mucate alone or in a formulation containing one or more excipients. For example, the composition at 5 minutes can exhibit greater than 55%, greater than 50%, greater than 45%, greater than 40%, greater than 35%, greater than 30%, or greater than 25% dissolution when mixed with 100 mL of pH 4.5 sodium acetate buffer at 37.0±0.5° C. For example, the composition at 10 minutes can exhibit greater than 80%, greater than 75%, greater than 65%, greater than 60%, greater than 55%, greater than 50%, dissolution when mixed with 100 mL of pH 4.5 sodium acetate buffer at 37.0±0.5° C. For example, the composition at 240 minutes can exhibit greater than 80%, greater than 75%, greater than 65%, greater than 60%, greater than 55%, greater than 50%, dissolution when mixed with 100 mL of pH 4.5 sodium acetate buffer at 37.0±0.5° C.

Mannitol is capable of crystallizing in three polymorphic states: α, β, and δ. These three forms can be distinguished by X-ray powder diffraction (XRPD), and each polymorph has a different melting point. See, e.g., Sharma and Kalonia, AAPS PharmaSciTech 5(1):E10 (2004). Even more surprising than the observation of a first eutectic with racemic isometheptene mucate or (R)-isometheptene mucate and mannitol (β polymorph) was the observation of a second eutectic with a different polymorphic form of mannitol (δ polymorph). The eutectic comprising δ mannitol and racemic isometheptene mucate or (R)-isometheptene mucate (also referred to herein as the “δ mannitol eutectic”) has several advantages over the eutectic comprising β mannitol and racemic isometheptene mucate or (R)-isometheptene mucate (also referred to herein as the “β mannitol eutectic”). Prime among these are a lower melting point than the β mannitol eutectic with (R)-isometheptene mucate (m.p.=134°±1° C.).

In some embodiments, the invention provides a eutectic pharmaceutical composition comprising racemic isometheptene mucate and mannitol or (R)-isometheptene mucate and mannitol, wherein the mannitol is in its β polymorphic state. In some embodiments, the invention provides a eutectic pharmaceutical composition comprising racemic isometheptene mucate and mannitol or (R)-isometheptene mucate and mannitol, wherein the mannitol is in its δ polymorphic state. In certain embodiments, the pharmaceutical composition comprising the mannitol in its β polymorphic state is a sublingual composition. In certain embodiments, the pharmaceutical composition comprising the mannitol in its β polymorphic state is an oral composition. In certain embodiments, the pharmaceutical composition comprising the mannitol in its δ polymorphic state is a sublingual composition. In certain embodiments, the pharmaceutical composition comprising the mannitol in its δ polymorphic state is an oral composition.

In some embodiments, the invention provides a composition comprising eutectic of mannitol and racemic isometheptene mucate. In some embodiments, the invention provides a composition comprising eutectic of mannitol and (R)-isometheptene mucate. The skilled worker will understand that these compositions may be suitable for administration in a variety of ways, such as those described herein. For example, a composition may be suitable for administration orally (administration wherein the racemic isometheptene or (R)-isometheptene is absorbed in the gastrointestinal tract), or for transmucosal absorption (e.g., sublingual, buccal, or intranasal absorption, or by inhalation).

### Methods of Manufacturing Eutectic Compositions

The skilled worker will appreciate that a eutectic composition of the invention can be manufactured according to any of a number of known methods. In some embodiments, the invention provides methods for producing a eutectic composition of the invention comprising milling an active pharmaceutical ingredient (API) (e.g., racemic isometheptene mucate or (R)-isometheptene mucate) with mannitol, mixing an API (e.g., racemic isometheptene mucate or (R)-isometheptene mucate) with mannitol, or a combination thereof. For example, the API and mannitol can be milled in an agate mortar or mixed in a high shear granulator. High shear mixing combines dry powders using a high speed impellor and chopper blades to uniformly mix the ingredients. Some particle size reduction is possible due to the shear force and the high speed of the mixing blades. The API and mannitol also can be milled and mixed in a Turbula® Shaker-Mixer. In certain embodiments, the API and mannitol can be mixed via compression, for example, via roller compaction. Roller compaction forces fine powders between two counter-rotating rolls and presses the raw materials into a solid compact or sheet (referred to as flakes). The flakes are reduced in size until they reach a desired grain size. In certain embodiments, a small amount of water is added during milling. In certain embodiments, mannitol can be melted and mixed with racemic isometheptene mucate or (R)-isometheptene mucate to form a eutectic composition. In certain embodiments, the API is a micronized API (e.g., micronized racemic isometheptene mucate or micronized (R)-isometheptene mucate). Without wishing to be bound by theory, the above methods of manufacturing can afford a eutectic comprising β-mannitol.

In some embodiments, the invention provides methods for producing a eutectic composition of the invention comprising spray drying a solution of an API (e.g., racemic isometheptene mucate or (R)-isometheptene mucate) with mannitol. The skilled worker will appreciate that spray drying is routine, and parameters for spray drying can be determined without undue experimentation. For example, spray drying can be performed under any of the following conditions:

### T Inlet (° C.): 120

### T Outlet (° C.): 73-76

Feed rate (ml/min): 4

### Flow Rate (L/h): 600

### Aspiration (100%): 100

delta Pressure (mbar): 2-10

These conditions also may be scaled up to provide higher throughput manufacturing. Without wishing to be bound by theory, spray drying can afford a eutectic comprising δ-mannitol.

### Methods of Detecting Eutectic Compositions

Methods of detecting eutectic compositions are well known. The skilled worker will appreciate that eutectic compositions can be detected by any of these methods. For example, rapid differential scanning calorimetry (“DSC”) can be used to detect a eutectic melting point by evaluating the amount of heat recorded from eutectic melting and comparing it with the melting heat of the eutectic composition. During a slow scan of DSC, the increased temperature in the crucible facilitates the formation of the eutectic even when the two components (such as mannitol and racemic isometheptene mucate may not have been mixed before the start of the experiment.) In contrast, a rapid DSC scan reduces the time during which eutectic compositions can form in the crucible because the temperature inside the crucible rapidly increases during the analysis and rapidly reaches the values at which the mannitol melts. Another useful method is measuring compaction force vs. DSC eutectic melting point. In this method, mixtures are prepared with known ratios and then submitted to well-defined compaction forces. DSC analyses are then performed and the heat of the eutectic melting versus the forces is then recorded and plotted. These values are compared with those obtained with the eutectic ratio, providing the percentage of eutectic in the formulation.

An additional method that can be used to detect the amount of eutectic in a composition is to compare tensile strength and compression force. In this method, tablets are prepared with only mannitol and API at different compression forces. For each tablet prepared, the percentage of eutectic formed versus tensile strength of the tablets is correlated. There is a proportionally linear correlation between the tensile strength and the intimate contact area. The slope of this correlation provides the percentage of the eutectic formed.

There is a linear correlation between the percentage of eutectic composition in a preparation and the porosity of powders in a composition. In this method, a standard curve can be generated by preparing samples with different ratios of components in which at least one of the components has a variety of different particle sizes, measuring the specific surface area and the porosity of the powders and plotting porosity against the percentage of eutectic. Because there is a linear correlation between the two parameters, the slope of this correlation with what is recorded for the eutectic mixture provides the percentage of the eutectic formed.

Dissolution rate also can be used to detect the percent of eutectic because a eutectic may have higher dissolution and higher bioavailability. In this method, the intrinsic dissolution rate (using disk sample holder in a defined and appropriate medium) of the single components is calculated, followed by the dissolution rate of the eutectic mixture. Based on the thermodynamic parameters (entropy), the eutectic should have a more rapid dissolution rate than the other mixtures. By these analyses, it is also possible to obtain information on the performance of a tablet in terms of bioavailability. This approach also can evaluate the higher bioavailability of a eutectic versus mixtures of the individual components.

Scanning Electron Microscopy (SEM) can be used by performing a scanning EM of each pure component, on the eutectic, and on the mixtures, and observing the different crystal morphology by pointing out the differently shaped particles.

### Methods of Administering Eutectic Compositions

Appropriate methods of administering a pharmaceutical composition of the invention to a subject will depend, for example, on the age of the subject, whether the subject is active or inactive at the time of administering, whether the subject is experiencing symptoms of a disease or condition at the time of administering, the extent of the symptoms, and the chemical and biological properties of the API (e.g. solubility, digestibility, bioavailability, stability and toxicity). In some embodiments, the pharmaceutical composition is administered for oral or transmucosal absorption.

Methods of administering compositions for oral absorption are well known in the art. For example, a composition may be administered orally through tablets, capsules, pills, or powders. In these embodiments, the compositions are absorbed by the gastrointestinal tract after swallowing. In certain embodiments, the composition lacks a film or membrane (e.g., a semipermeable membrane).

Methods of administering compositions for transmucosal absorption are well known in the art. For example, a composition may be administered for buccal absorption through buccal tablets, lozenges, buccal powders, and buccal spray solutions. A composition may be administered for sublingual absorption through sublingual tablets, sublingual films, liquids, sublingual powders, and sublingual spray solutions. In certain embodiments, the composition lacks a film or membrane (e.g., a semipermeable membrane). A composition may be administered for intranasal absorption through nasal sprays. A composition may be administered for pulmonary absorption through aerosolized compositions and inhalable dried powders. Because mannitol powder is an inhalation product in the U.S. (trade name: Aridol®; Pharmaxis Ltd.), inhalation may be an especially beneficial form of administration. When administered via sprays or aerosolized compositions, a composition may be prepared with saline as a solution, employ benzyl alcohol or other suitable preservatives, or include absorption promoters to enhance bioavailability, fluorocarbons, and/or other solubilizing or dispersing agents.

Doses and dosing regimens can be determined by one of skill in the art according to the needs of a subject to be treated. The skilled worker may take into consideration factors such as the age or weight of the subject, the severity of the disease or condition being treated, and the response of the subject to treatment. A composition of the invention can be administered, for example, as needed or on a daily basis. In some embodiments, a composition can be administered immediately prior to sleep or several hours before sleep. Administration prior to sleep may be beneficial by providing the therapeutic effect before the onset of the symptoms of the disease or condition being treated. Dosing may take place over varying time periods. For example, a dosing regimen may last for 1 week, 2 weeks, 3 weeks, 4 weeks, 5 weeks, 6 weeks, 7 weeks, 8 weeks, 9 weeks, 10 weeks, 11 weeks, 12 weeks, or longer. In some embodiments, a dosing regimen will last 1 month, 2 months, 3 months, 4 months, 5 months, 6 months, 7 months, 8 months, 9 months, 10 months, 11 months, 12 months, or longer.

### Therapeutic Uses

### Analgesic

The pharmaceutical compositions of these inventions act as potent and selective pain inhibitors to treat, ameliorate, reduce the severity of, or prevent diseases or disorders, including, but not limited to, inflammatory diseases, allergic diseases, allodynia, fibromyalgia, migraine, and rheumatoid arthritis.

### Pain

The pharmaceutical compositions of these inventions may be used in the treatment, therapy, or prevention of pain. Pain is an unpleasant feeling triggered by the nervous system. It is often classified by the region of the body involved, the system whose dysfunction may be causing the pain, the duration and pattern of occurrence, the intensity and time since onset, and the etiology. Many types of pain exist, including, but not limited to, nociceptive pain, neuropathic pain, psychogenic pain, visceral pain, and chronic pain.

### Headaches and Episodic Tension-Type Headaches

In some embodiments, pharmaceutical compositions of these inventions may be used in treatment, therapy, or prevention of pain caused by headaches and episodic tension-type headaches. A headache is pain in any region of the head, and may occur on one or both sides of the head, be isolated to a certain location, radiate across the head from one point, or have a vise-like quality. Headaches can cause sharp pain, a throbbing sensation, or a dull ache. Primary headaches can be caused by problems with or overactivity of pain-sensitive structures in the head, and secondary headaches can be caused by diseases, such as brain cancer, glaucoma, and trigeminal neuralgia, which activate the pain-sensitive nerves in the head. A tension-type headache is classified into subtypes based on how often it occurs: infrequent episodic tension-type headache (ETTH) (<1 day/month on average), frequent ETTH (1-14 days/month on average), or chronic TTH, or CTTH, (˜15 days/month on average). An ETTH (infrequent or frequent) may be described as a mild to moderate constant band-like pain, tightness, or pressure around the forehead or back of the head and neck. ETTH may last from 30 minutes to several days. ETTH usually begins gradually, and often occurs in the middle of the day. The severity of a tension headache generally increases significantly with its frequency. Because the symptoms of ETTH overlap with other primary headache types, diagnosis is generally made, not only by inclusion, but also of exclusion of certain symptoms such as nausea, exacerbation by physical exercise and occurrence of both photophobia and phonophobia.

### Migraine

In some embodiments, pharmaceutical compositions of these inventions may be used in treatment, therapy, or prevention of migraines; tension or migraine headaches due to a vascular, neurovascular, or neurogenic disorder or dysfunction during a menstrual cycle episode; or tension or migraine headaches due to a vascular, neurovascular, or neurogenic disorder or dysfunction during a migraine episode. Migraine is described as a paroxysmal disorder or a recurrent, incapacitating, neurovascular disorder characterized by unilateral and throbbing headaches associated characterized by attacks of headache, nausea, vomiting, photophobia, and phonophobia.

Migraine affects people of all races and both sexes with women accounting for 79% (61% between 20 and 49 years of age) of physician visits for migraines and Caucasians for 91% of the physician visits. Migraine without aura often has a strict menstrual relationship. The pathogenesis of migraine headache involves a) the cranial blood vessels, b) the trigeminal innervation of these vessels, and c) the reflex connection of the trigeminovascular system in the cranial parasympathetic outflow.

Migraine pathophysiology is believed by genetic predisposition to involve leakage of ion channels in the brain stem such that the decreased blood flow in the brain leads to neuropeptide release from trigeminal nerves inducing dilatation of cranial extracerebral blood vessel. This condition stimulates the trigeminovascular system producing headache associated phonophobia and photophobia as well as nausea and vomiting.

### Phantom Limb Pain

In some embodiments, pharmaceutical compositions of these inventions may be used in treatment, therapy, or prevention of phantom pain. Phantom pain is pain coming from a body part that's no longer there. This pain originates in the spinal cord and brain and may be described as shooting, stabbing, boring, squeezing, throbbing or burning.

### Depression

Compositions of these inventions may be used in treatment, therapy, or prevention of depression. Depression, clinical depression, major depression, unipolar depression, unipolar disorder, or recurrent depression in the case of repeated episodes is a psychiatric diagnosis for a mood disorder characterized by episodes of all encompassing low mood accompanied by low self-esteem and loss of interest or pleasure in normally enjoyable activities (anhedonia) and disturbed sleep (typically early morning awakening). The term “depression” is ambiguous and can be used to describe manic-depressive disorder, but is also used to describe other mood disorders or to lower mood states lacking clinical significance. For example, endogenous depression or the depressed phases of bipolar disorder can be associated with widespread pain or regional pain disorders.

Pain experienced during depression can include, but is not limited to, psychogenic pain, psychiatric pain, psychic pain, and psychological pain. Psychogenic pain is pain that results from psychological mechanisms including traumatic experiences, empathic reactions or somatization. For example, loss of a loved friend or relative by death or other separation can result in widespread pain, regional pain, and other symptoms including reactive depression. Psychiatric pain is pain that results from conditions that are believed to have biological causes. Psychic pain and psychological pain are caused by a non-physical origin and can lead to emotional suffering and mental agony.

### Allodynia

Compositions of these inventions may be used in treatment, therapy, or prevention of allodynia. Allodynia, or pain due to a stimulus that does not usually provoke pain, is a prominent symptom in patients with neuropathic pain. Allodynia is seen in various peripheral neuropathies and central pain disorders, and affects 15-50% of patients with neuropathic pain. Allodynia is classified according to the sensory modality (touch, pressure, pinprick, cold, and heat) that is used to elicit the sensation.

### Fibromyalgia

Fibromyalgia is a disorder characterized by widespread musculoskeletal pain accompanied by fatigue, sleep, memory and mood issues. Research indicates that fibromyalgia amplifies painful sensations by affecting the way the brain processes pain signals. Symptoms of fibromyalgia sometimes begin after a physical trauma, surgery, infection, or significant psychological stress. In other cases, symptoms gradually accumulate over time with no single triggering event. Symptoms include: widespread pain on both sides of the body and above and below the waist, fatigue, cognitive difficulties, depression, headaches, and pain or cramping in the lower abdomen. Compositions of these inventions may be used in treatment, therapy, or prevention of fibromyalgia.

### Fibromyalgia-ness

Fibromyalgia-ness is the tendency to respond to illness and psychosocial stress with fatigue and widespread pain. Compositions of these inventions may be used in treatment, therapy, or prevention of fibromyalgia-ness.

### Central Sensitization

Compositions of these inventions may be used in treatment, therapy, or prevention central sensitization. Central or chronic sensitization is a condition of the nervous system that is associated with the development and maintenance of chronic pain. When central sensitization occurs, the nervous system goes through a process called “wind-up” and gets regulated in a persistent state of high reactivity. This persistent, or regulated, state of reactivity subsequently comes to maintain pain even after the initial injury might be healed.

Central sensitization has two main characteristics. Both involve a heightened sensitivity to pain and the sensation of touch. They are called ‘allodynia’ and ‘hyperalgesia.’ Allodynia occurs when a person experiences pain with things that are normally not painful. Hyperalgesia occurs when an actual painful stimulus is perceived as more painful than it should. With allodynia and hyperalgesia, the sensation of pain travels through the nervous system, which is in a persistent state of high reactivity, and the pain is registered in the brain as a heightened level of pain.

### Centralization

Compositions of these inventions may be used in treatment, therapy, or prevention of centralization. The pathogenesis of fibromyalgia is believed to involve sensitization of the central nervous system (CNS) to perceiving painful stimuli, which is termed “central sensitization” or “centralization”. Centralization leads to the perception of widespread pain. Pain of this type is termed, “central neuropathic pain” or “central pain”. Centralization also leads and to other symptoms, including visceral pain such as irritable bowel, tension-type headache, and migraine.

### Regional Pain Syndrome

A composition of these inventions may be therapeutic for regional pain syndrome. Regional pain syndrome or complex regional pain syndrome (CRPS) is a chronic pain condition most often affecting one of the limbs (arms, legs, hands, or feet), usually after an injury or trauma to that limb. CRPS is believed to be caused by damage to, or malfunction of, the peripheral and central nervous systems. CRPS is characterized by prolonged or excessive pain and mild or dramatic changes in skin color, temperature, and/or swelling in the affected area.

### Temporomandibular Joint Syndrome (TMJ)

A composition of these inventions may be therapeutic for temporomandibular joint syndrome (TMJ). TMJ disorders can cause pain in the jaw joint and in the muscles that control jaw movement. Signs and symptoms of TMJ disorders may include: pain or tenderness of the jaw, aching pain in and around the ear, difficulty chewing or discomfort while chewing, aching facial pain, locking of the jaw joint, and a clicking sound or grating sensation when opening the mouth or chewing.

### Lower Back Pain

Lower back pain may be dull or sharp pain in the lower back. The pain may be in one small area or over a broad area and may include muscle spasms. Lower back pain may be caused by overuse, strain, or injury; aging; a herniated disc; arthritis; compression fractures; illness; a congenital spine problem; or other causes. A composition of these inventions may be therapeutic for lower back pain.

### Gulf War Syndrome

A prominent condition affecting Gulf War Veterans is a cluster of medically unexplained chronic symptoms that can include fatigue, headaches, joint pain, indigestion, insomnia, dizziness, respiratory disorders, and memory problems. In certain embodiments, a compound or composition of these inventions may be used in treatment or therapy for Gulf War syndrome.

### Visceral Pain

In certain embodiments, a composition of these inventions may be used in treatment, therapy, or prevention of visceral pain. Visceral pain is caused by the activation of pain receptors in the chest, abdomen, or pelvic areas. Visceral pain is caused by problems with internal organs, such as the stomach, kidney, gallbladder, urinary bladder, and intestines. These problems include distension, perforation, inflammation, and impaction or constipation, which can cause associated symptoms, such as nausea, fever, malaise, and pain. Visceral pain is also caused by problems with abdominal muscles and the abdominal wall, such as spasm. Visceral pain is vague and not well localized and is usually described as pressure-like, deep squeezing, dull, or diffuse.

### Neuropathic Pain

Neuropathic pain is a complex, chronic pain state that usually is accompanied by tissue injury. With neuropathic pain, the nerve fibers themselves might be damaged, dysfunctional, or injured, and these damaged nerve fibers send incorrect signals to other pain centers. The impact of a nerve fiber injury includes a change in nerve function both at the site of injury and areas around the injury. In certain embodiments, a composition of these inventions may be used to alleviate or prevent neuropathic pain.

### Sickle Cell Pain

In some embodiments, a composition of these inventions may be used in treatment, therapy, or prevention of sickle cell pain. Sickle cell disease causes red blood cells to form into a crescent shape, like a sickle. The sickle-shaped red blood cells break apart easily, causing anemia, and the damaged sickle red blood cells clump together and stick to the walls of blood vessels, blocking blood flow. This can cause severe pain and permanent damage to the brain, heart, lungs, kidneys, liver, bones, and spleen.

### Nociceptive Pain

Nociceptive pain is caused when special nerve endings—called nociceptors—are irritated. Nociceptors are the nerves which sense and respond to parts of the body which suffer from damage. They signal tissue irritation, impending injury, or actual injury. When activated, they transmit pain signals (via the peripheral nerves as well as the spinal cord) to the brain. The pain is typically well localized, constant, and often with an aching or throbbing quality. In some embodiments, a composition of these inventions may be used in treatment, therapy or prevention of nociceptive pain.

### Post-Operative Pain

Post-operative pain is pain that occurs after an operation. In some embodiments, a composition of these inventions may be used in treatment, therapy or prevention of post-operative pain.

### Orthopedic Injury Pain

Orthopedic injuries are conditions involving the musculoskeletal system, and can include musculoskeletal trauma, sports injuries, degenerative diseases, or infections. Pain caused by orthopedic injury may be treated or prevented by compositions of these inventions.

### Osteoarthritis

Osteoarthritis is the most common form of arthritis, affecting millions of people worldwide. It occurs when the protective cartilage on the ends of the bones wears down over time. Symptoms include: pain, tenderness, stiffness, loss of flexibility, grating sensation, and bone spurs. In some embodiments, a composition of these inventions may be used in treatment or therapy for osteoarthritis.

### Rheumatoid Arthritis

In some embodiments, a composition of these inventions may be used in treatment, therapy, or prevention of rheumatoid arthritis. Rheumatoid arthritis is a chronic inflammatory disorder that typically affects the small joints in the hands and feet. Rheumatoid arthritis affects the lining of the joints, causing painful swelling that can eventually result in bone erosion and joint deformity. An autoimmune disorder, rheumatoid arthritis occurs when the immune system mistakenly attacks the body's own tissues. In addition to causing joint problems, rheumatoid arthritis sometimes can affect other organs of the body—such as the skin, eyes, lungs, and blood vessels. Signs and symptoms of rheumatoid arthritis may include: tender, warm, swollen joints; morning stiffness; rheumatoid nodules; and fatigue, fever, and weight loss.

Pain Associated with Post-Traumatic Stress Disorder (PTSD)

In some embodiments, a composition of these inventions may be used in treatment, therapy, or prevention of pain associated with post-traumatic stress disorder (PTSD). PTSD is a mental health condition that's triggered by a terrifying event—either experiencing it or witnessing it. Symptoms may include chronic pain, flashbacks, nightmares, and severe anxiety, as well as uncontrollable thoughts about the event.

Merely to illustrate some embodiments of the invention, “treatment” of a migraine headache may include an improvement in any of the following symptoms or conditions associated with migraine headache (or combination thereof): pain on one side or both sides of the head, sensitivity to light and sounds, nausea and vomiting, blurred vision, allodynia, and lightheadness. “Treatment” of pain may include a reduction in the pain experienced by the patient. “Treatment” of fibromyalgia may include an improvement in any of the following symptoms or conditions associated with fibromyalgia (or combination thereof): widespread pain, fatigue, and cognitive difficulties (e.g., impaired ability to focus). “Treatment” of a headache or an episodic tension-type headache may include an improvement in any of the following symptoms or conditions associated with a headache or an episodic tension-type headache (or combination thereof): sharp pain, throbbing sensation, dull ache, and nausea. “Treatment” of phantom limb pain may include an improvement in any of the following symptoms or conditions associated with phantom limb pain (or combination thereof): shooting, stabbing, or squeezing pain coming from the body part that is no longer there. “Treatment” of depression may include an improvement in any of the following symptoms or conditions associated with depression (or combination thereof): unexplained aches and pains, concentration problems, loss of energy, and anger or irritability. “Treatment” of psychic or psychological pain may include an improvement in any of the following symptoms or conditions associated with psychic or psychological pain (or combination thereof): emotional suffering and mental agony. “Treatment” of psychiatric pain may include an improvement in any of the following symptoms or conditions associated with psychiatric pain (or combination thereof): widespread pain and regional pain. “Treatment” of allodynia may include an improvement in any of the following symptoms or conditions associated with a symptom related to allodynia (or combination thereof): pain due to a stimulus that does not usually provoke pain. “Treatment” of fibromyalgia-ness may include an improvement in any of the following symptoms or conditions associated with a symptom related to fibromyalgia-ness (or combination thereof): fatigue and widespread pain. “Treatment” of central sensitization may include an improvement in any of the following symptoms or conditions associated with a symptom related to central sensitization (or combination thereof): allodynia and hyperalgesia. “Treatment” of centralization may include an improvement in any of the following symptoms or conditions associated with a symptom related to centralization (or combination thereof): irritable bowel, tension-type headache, and migraine. “Treatment” of regional pain syndrome may include an improvement in any of the following symptoms or conditions associated with a symptom related to regional pain syndrome (or combination thereof): swelling and pain in the arms, legs, hands, or feet. “Treatment” of temporomandibular joint syndrome (TMJ) may include an improvement in any of the following symptoms or conditions associated with a symptom related to TMJ (or combination thereof): pain or tenderness of the jaw, aching pain in and around the ear, difficulty chewing or discomfort while chewing, aching facial pain, locking of the jaw joint, and a clicking sound or grating sensation when opening the mouth or chewing. “Treatment” of lower back pain may include an improvement in any of the following symptoms or conditions associated with a symptom related to lower back pain (or combination thereof): pain in the lower back and muscles spasms in the lower back. “Treatment” of Gulf War syndrome may include an improvement in any of the following symptoms or conditions associated with a symptom related to Gulf War syndrome (or combination thereof): fatigue, headaches, and joint pain. “Treatment” of visceral pain may include an improvement in any of the following symptoms or conditions associated with a symptom related to visceral pain (or combination thereof): pressure-like, deep squeezing, dull, or diffuse pain in the chest, abdomen, or pelvic areas. “Treatment” of neuropathic pain may include an improvement in any of the following symptoms or conditions associated with a symptom related to neuropathic pain (or combination thereof): shooting and burning pain, tingling, and numbness. “Treatment” of sickle cell pain may include an improvement in any of the following symptoms or conditions associated with a symptom related to sickle cell pain (or combination thereof): pain in the chest, abdomen, joints, and bones. “Treatment” of nociceptive pain may include an improvement in any of the following symptoms or conditions associated with a symptom related to nociceptive pain (or combination thereof): aching or throbbing pain. “Treatment” of post-operative pain may include an improvement in any of the following symptoms or conditions associated with a symptom related to post-operative pain (or combination thereof): pain, swelling, and irritation after an operation. “Treatment” of orthopedic injury pain may include an improvement in any of the following symptoms or conditions associated with a symptom related to orthopedic injury pain (or combination thereof): pain, swelling, and irritation after an orthopedic injury. “Treatment” of osteoarthritis may include an improvement in any of the following symptoms or conditions associated with a symptom related to osteoarthritis (or combination thereof): pain, tenderness, stiffness, loss of flexibility, grating sensation, and bone spurs. “Treatment” of rheumatoid arthritis may include an improvement in any of the following symptoms or conditions associated with a symptom related to rheumatoid arthritis (or combination thereof): tender, warm, swollen joints; morning stiffness; rheumatoid nodules; and fatigue, fever and weight loss. “Treatment” of pain associated with post-traumatic stress disorder (PTSD) may include an improvement in any of the following symptoms or conditions associated with a symptom related to pain associated with post-traumatic stress disorder (PTSD) (or combination thereof): chronic pain and headaches.

Improvements in any of these symptoms can be readily assessed according to standard methods and techniques known in the art. Symptoms are not limited to those listed above and other symptoms may also be monitored in order to determine the effectiveness of treatment. The population of subjects treated by the method of the inventions includes subjects suffering from the undesirable condition or disease, as well as subjects at risk for development of the condition or disease. Without wishing to be bound by theory, in certain embodiments, administering any of the compositions described herein may have any one or more of the following effects: analgesia; alleviation of widespread pain; decrease in pain from headaches, tension-type headaches, and migraines; and relief of pain associated with PTSD, rheumatoid arthritis, allodynia, fibromyalgia, and fibromyalgia-ness. It should be noted that any of the compositions described above or herein may be used in any of the methods described herein. Effects are not limited to those listed above and other effects may also be noted during treatment.

The terms “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition or prevents the unwanted condition, whereas if it is administered after manifestation of the unwanted condition, the treatment is therapeutic (i.e., it is intended to diminish, ameliorate, or stabilize the existing unwanted condition or side effects thereof).

The terms “therapeutic agent”, “drug”, “medicament” and “bioactive substance” are art-recognized terms and include molecules and other agents that are biologically, physiologically, or pharmacologically active substances that act locally or systemically in a patient or subject to treat a disease or condition.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular composition without necessitating undue experimentation. In certain embodiments, a therapeutically effective amount of a therapeutic agent for in vivo use will likely depend on a number of factors, including: the identity of the agent and the mode and method of administration.

As used herein. the term “therapeutically effective dose” refers to a dose that produces the desired effect for which it is administered. The exact dose will depend on the purpose of the treatment, and will be ascertainable by one skilled in the art using known techniques (see, e.g., Lloyd (1999) The Art, Science and Technology of Pharmaceutical Compounding).

### Animal Models

Pain, headaches, and migraines have been modeled in animals such as mice and rats. For example, Oshinsky et al. (Oshinsky, M. L., et al., Spontaneous Trigeminal Allodynia in Rats: A Model of Primary Headache, 2012, 52: 1336-1349) describes spontaneous trigeminal allodynia (STA) rats with the inherited trait of spontaneously changing trigeminal von Frey thresholds. These rats are a model of spontaneous headache and can be used as a model of primary headache. Through a series of tactile sensory tests, the periorbital, hind-paw, and jaw-pressure thresholds for STA rats are determined by applying von Frey monofilaments. These determinations are made both before and after receiving treatments with compositions of interest. Analgesic activity of the compositions described herein can be evaluated by determining trigeminal von Frey thresholds in STA rats.

Common mouse models for pain include the Formalin Test (Wheeler-Aceto, et al., Psychopharmacology, 104, 35-44, 1991), the Hot Plate Test (Eddy and Leimbach, J. Pharmacol. Exp. Ther., 107, 385-393, 1953), and the Tail-flick Test (D'Amour and Smith, J. Pharmacol. Exp. Ther., 1, 74-79, 1941). These methods detect analgesic activity of compositions of interest. In the Formalin Test, mice are given an intraplantar injection of 5% formalin into one posterior hindpaw to induce paw licking. Test compositions are given to the mice before treatment with formalin and the mice are evaluated and compared to a control group. In the Hot Plate Test, mice are placed onto a hot metal plate maintained at 54° C. and the latency to the first foot-lick is measured. As with the Formalin test, compositions of interest are given to the mice before the test and the mice are evaluated and compared to a control group. In the Tail-flick Test, a mouse's tail is heated by means of a thermal light source, and the latency before the animal withdraws its tail is measured. Test compositions are administered before the test, and compared with a vehicle control group. The analgesic activity of the compositions described herein can be identified using the mouse formalin, hot plate, and tail-flick tests.

### Excipients

In some embodiments, a composition of the invention is useful as a medicament. In some embodiments, the invention provides for the use of a composition of the invention in the manufacture of a medicament. In some embodiments, it may be beneficial to include one or more excipients in the compositions of the invention. One of skill in the art would appreciate that the choice of any one excipient may influence the choice of any other excipient. For example, the choice of a particular excipient may preclude the use of one or more additional excipient because the combination of excipients would produce undesirable effects. One of skill in the art would be able to empirically determine which additional excipients, if any, to include in the formulations of the invention. For example, racemic isometheptene mucate or (R)-isometheptene mucate can be combined with at least one pharmaceutically acceptable carrier such as a solvent, bulking agents, binder, humectant, disintegrating agent, solution retarder, disintegrant, glidant, absorption accelerator, wetting agent, solubilizing agent, lubricant, sweetening agent, or flavorant agent. A “pharmaceutically acceptable carrier” refers to any diluent or excipient that is compatible with the other ingredients of the formulation, and which is not deleterious to the recipient. A pharmaceutically acceptable carrier can be selected on the basis of the desired route of administration, in accordance with standard pharmaceutical practices.

### Bulking Agents

In some embodiments, it may be beneficial to include a bulking agent in the compositions of the invention. Bulking agents are commonly used in pharmaceutical compositions to provide added volume to the composition. Bulking agents are well known in the art. Accordingly, the bulking agents described herein are not intended to constitute an exhaustive list, but are provided merely as exemplary bulking agents that may be used in the compositions and methods of the invention.

Exemplary bulking agents may include carbohydrates, sugar alcohols, amino acids, and sugar acids. Bulking agents include, but are not limited to, mono-, di-, or poly-, carbohydrates, starches, aldoses, ketoses, amino sugars, glyceraldehyde, arabinose, lyxose, pentose, ribose, xylose, galactose, glucose, hexose, idose, mannose, talose, heptose, glucose, fructose, methyl u-D-glucopyranoside, maltose, lactone, sorbose, erythrose, threose, arabinose, allose, altrose, gulose, idose, talose, erythrulose, ribulose, xylulose, psicose, tagatose, glucosamine, galactosamine, arabinans, fructans, fucans, galactans, galacturonans, glucans, mannans, xylans, inulin, levan, fucoidan, carrageenan, galactocarolose, pectins, amylose, pullulan, glycogen, amylopectin, cellulose, microcrystalline cellulose, pustulan, chitin, agarose, keratin, chondroitin, dermatan, hyaluronic acid, xanthin gum, sucrose, trehalose, dextran, lactose, alditols, inositols, sorbitol, mannitol, glycine, aldonic acids, uronic acids, aldaric acids, gluconic acid, isoascorbic acid, ascorbic acid, glucaric acid, glucuronic acid, gluconic acid, glucaric acid, galacturonic acid, mannuronic acid, neuraminic acid, pectic acids, maize starch, isomalt, and alginic acid.

### Disintegrants

In some embodiments, it may be beneficial to include a disintegrant in the compositions of the invention. Disintegrants aid in the breakup of solid compositions, facilitating delivery of an active pharmaceutical composition. Disintegrants are well known in the art. Some disintegrants have been referred to as superdisintegrants because they have fast properties, and may be used as disintegrants in the context of the invention. Accordingly, the disintegrants described herein are not intended to constitute an exhaustive list, but are provided merely as exemplary disintegrants that may be used in the compositions and methods of the invention. Exemplary disintegrants include crospovidone, povidone, plasdone, microcrystalline cellulose, sodium carboxymethyl cellulose, methyl cellulose, sodium starch glycolate, calcium carboxymethyl croscarmellose sodium, polyvinylpyrrolidone, lower alkyl-substituted hydroxypropyl cellulose, Indion 414, starch, pre-gelatinized starch, calcium carbonate, gums, sodium alginate, Ac-Di-Sol, and Pearlitol Flash®. Pearlitol Flash® (Roquette) is a mannitol-maize starch disintegrant that is specifically designed for orally dispersible tablets (ODT). Certain disintegrants have an effervescent quality.

### Glidants

In some embodiments, it may be beneficial to include a glidant in the compositions of the invention. Glidants aid in the ability of a powder to flow freely. Glidants are well known in the art. Accordingly, the glidants described herein are not intended to constitute an exhaustive list, but are provided merely as exemplary glidants that may be used in the compositions and methods of the invention. Exemplary glidants include colloidal silica (silicon dioxide), magnesium stearate, starch, talc, glycerol behenate, DL-leucine, sodium lauryl sulfate, calcium stearate, and sodium stearate.

### Lubricants

In some embodiments, it may be beneficial to include a lubricant in the compositions of the invention. Lubricants help keep the components of a composition from clumping. Lubricants are well known in the art. Accordingly, the lubricants described herein are not intended to constitute an exhaustive list, but are provided merely as exemplary lubricants that may be used in the compositions and methods of the invention. Exemplary lubricants include calcium stearate, magnesium stearate, stearic acid, sodium stearyl fumarate, vegetable based fatty acids, talc, mineral oil, light mineral oil, hydrogenated vegetable oil (e.g., peanut oil, cottonseed oil, sunflower oil, sesame oil, olive oil, corn oil, safflower oil, canola oil, coconut oil and soybean oil), silica, zinc stearate, ethyl oleate, ethyl laurate.

### Sweeteners

In some embodiments, it may be beneficial to include a sweetener in the compositions of the invention. Sweeteners help improve the palatability of the composition by conferring a sweet taste to the composition. Sweeteners are well known in the art. Accordingly, the sweeteners described herein are not intended to constitute an exhaustive list, but are provided merely as exemplary sweeteners that may be used in the compositions and methods of the invention. Exemplary sweeteners include, without limitation, compounds selected from the saccharide family such as the mono-, di-, tri-, poly-, and oligosaccharides; sugars such as sucrose, glucose (corn syrup), dextrose, invert sugar, fructose, maltodextrin and polydextrose; saccharin and salts thereof such as sodium and calcium salts; cyclamic acid and salts thereof; dipeptide sweeteners; chlorinated sugar derivatives such as sucralose and dihydrochalcone; sugar alcohols such as sorbitol, sorbitol syrup, xylitol, hexa-resorcinol, and the like, and combinations thereof. Hydrogenated starch hydrolysate, and the potassium, calcium, and sodium salts of 3,6-dihydro-6-methyl-1-1,2,3-oxathiazin-4-one-2,2-dioxide many also be used.

### Flavorants

In some embodiments, it may be beneficial to include a flavorant in the compositions of the invention. Flavorants help improve the palatability of the composition by conferring a more desirable taste to the composition. Flavorants are well known in the art. Accordingly, the flavorants described herein are not intended to constitute an exhaustive list, but are provided merely as exemplary flavorants that may be used in the compositions and methods of the invention. Exemplary flavorants include, without limitation, natural and/or synthetic (i.e., artificial) compounds such as mint, peppermint, spearmint, wintergreen, menthol, anise, cherry, strawberry, watermelon, grape, banana, peach, pineapple, apricot, pear, raspberry, lemon, grapefruit, orange, plum, apple, lime, fruit punch, passion fruit, pomegranate, chocolate (e.g., white, milk, dark), vanilla, caramel, coffee, hazelnut, cinnamon, combinations thereof, and the like.

### Coloring Agents

Coloring agents can be used to color code the composition, for example, to indicate the type and dosage of the therapeutic agent therein. Coloring agents are well known in the art. Accordingly, the coloring agents described herein are not intended to constitute an exhaustive list, but are provided merely as exemplary coloring agents that may be used in the compositions and methods of the invention. Exemplary coloring agents include, without limitation, natural and/or artificial compounds such as FD & C coloring agents, natural juice concentrates, pigments such as titanium oxide, silicon dioxide, and zinc oxide, combinations thereof, and the like.

### Combination Therapy

In some embodiments, the compositions of the inventions can be used in combination with other therapeutics as analgesics. In certain embodiments, the compositions of the inventions can be used in combination with other therapeutics to treat pain; an episodic tension-type headache; a migraine headache; a headache; psychic pain; psychological pain; psychiatric pain; depression; allodynia; fibromyalgia; fibromyalgia-ness; central sensitization; centralization; regional pain syndrome; temporomandibular joint syndrome (TMJ); lower back pain; Gulf War syndrome; visceral pain; neuropathic pain; sickle cell pain; nociceptive pain; post-operative pain; orthopedic injury pain; phantom limb pain; osteoarthritis; rheumatoid arthritis; or pain associated with post-traumatic stress disorder (PTSD).

In some embodiments, the phrase “combination therapy” refers to the administration of any of the compositions described herein and an additional therapeutic agent as part of a specific treatment regimen intended to provide a beneficial effect from the co-action of these therapeutic agents. Administration of these therapeutic agents in combination typically is carried out over a defined time period (usually minutes, hours, days or weeks depending upon the combination selected). In certain embodiments, “combination therapy” refers to administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally, or by intravenous injection. “Combination therapy” also can embrace the administration of the therapeutic agents as described above in further combination with other biologically active ingredients (such as, but not limited to, a second and different therapeutic agent) and non-drug therapies (such as, but not limited to, surgery or radiation).

In another example of combination therapy, one or more compositions described herein can be used as part of a therapeutic regimen combined with one or more additional treatment modalities. By way of example, such other treatment modalities include, but are not limited to, dietary therapy, occupational therapy, physical therapy, ventilator supportive therapy, massage, acupuncture, acupressure, mobility aids, assistance animals, speech therapy, language therapy, educational therapy, psychological therapy, occupational therapy, and the like.

In some embodiments, the mammalian disease treated by the combination therapy can include any of the conditions described herein. Besides being useful for human treatment, the combination therapy is also useful for veterinary treatment of companion animals, exotic and farm animals, including rodents, horses, dogs, and cats.

In other embodiments, the therapeutic agents administered in combination therapy with any of the compositions of these inventions can comprise: acetaminophen, non-steroidal anti-inflammatory drugs (NSAIDs), ibuprofen, naprosyn, cyclooxygenase-2 inhibitors, aspirin, caffeine, dichloralphenazone, triptans, antidepressants, serotonin-norepinephrine reuptake inhibitors (SNRIs), and gabapentinoids.

In other embodiments, the therapeutic agents administered in combination therapy with any of the compositions of these inventions can comprise: anti-inflammatory agents, corticosteroids, CYP2D6 inhibitors, and TNF-alpha inhibitors.

In certain embodiments, the therapeutic agents administered in combination therapy with the compositions of the inventions can comprise one or more opiates.

Anti-inflammatory agents include, but are not limited to, non-steroidal anti-inflammatory drugs (NSAIDs) such as ibuprofen and naprosyn (naproxen); TNF-α blockers or inhibitors such as infliximab, adalimumab, and etanercept; IL-RA; azathioprine; cyclophosphamide; sulfasalazine; cyclooxygenase-2 inhibitors such as aspirin; caffeine; acetaminophen; ketoprofen; dichloralphenzone, triptans such as sumatriptan succinate; dexibuprofen; fenoprofen; dexketoprofen; flurbiprofen; oxaprozin; loxoprofen; indomethacin; tolmetin; sulindac; droxicam; lornoxicam; isoxicam; mefenamic acid; cortisol; corticosteroids such as cortisone, hydrocortisone, prednisone, prednisolone, fludrocortisone, methylpredinisone, dexamethasone, betamethasone, and triamcinolone; and meclofenamic acid. Gabapentinoids include, but are not limited to, gabapentin, pregabalin, gabapentin enacarbil, atagabalin, 4-methylpregabalin, and PD-217,014.

Antidepressants include, but are not limited to, selective serotonin reuptake inhibitors (SSRIs) such as fluoxetine, paroxetine, sertraline, citalopram, and escitalopram; serotonin and norepinephrine reuptake inhibitors (SNRIs) such as duloxetine, venlafaxine, desvenlafaxine, tramadol, tapentadol, and levomilnacipran; norepinephrine and dopamine reuptake inhibitors (NDRIs) such as Bupropion; trazodone; mirtazapine; vortioxetine; vilazodone; tricyclic antidepressants such as imipramine, nortriptyline, amitriptyline, doxepin, trimipramine, desipramine, and protriptyline; and monoamine oxidase inhibitors (MAOIs) such as tranylcypromine, phenelzine, and isocarboxazid.

CYP2D6 inhibitors include, but are not limited to, fluoxetine, paroxetine, bupropion, quinidine, cinacalcet, ritonavir, sertraline, duloxetine, and terbinafine. Not to be bound by theory, but in some embodiments a compound of the invention is metabolized by CYP2D6. In such embodiments, a CYP2D6 inhibitor may slow metabolism of a compound of the invention.

Opiates include, but are not limited to, codeine, thebaine, hydrocodone, hydromorphone, morphine, oxycodone, oxymorphone, and tramadol.

Such combination products employ the compositions of this application within the dosage range described herein and the other pharmaceutically active compound or compounds within approved dosage ranges and/or the dosage described in the publication reference.

In some embodiments, any of the compositions described herein can allow the combination therapeutic agents and/or compositions described herein to be administered at a low dose, that is, at a dose lower than has been conventionally used in clinical situations.

Alternatively, the methods and combination of the inventions can also maximize the therapeutic effect at higher doses.

In some embodiments, when administered as a combination, the therapeutic agents can be formulated as separate compositions which are given at the same time or different times, or the therapeutic agents can be given as a single composition.

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention.

The following examples are set forth as being representative of the present invention. These examples are not to be construed as limiting the scope of the invention as these and other equivalent embodiments will be apparent in view of the present disclosure, figures, and accompanying claims.

### EXAMPLES

### Example 1

Thermal analytical techniques were used to assess the compatibility of a drug product containing racemic isometheptene mucate. The compatibility assessment was carried out between the racemic isometheptene mucate and a number of possible excipients in a 1:1 weight ratio. Based on the thermal events recorded for each component and for the mixtures, the analyses were carried out by investigating the peaks recorded by differential scanning calorimetry (DSC) in mixture between racemic isometheptene mucate and the excipients. Differences in thermal profiles between the single compound and the related mixture obtained after milling the products in an agate mortar were evaluated.

The following raw materials were used:

Racemic isometheptene mucate
Granulated racemic isometheptene mucate

### Mannitol SD₂₀₀

### Mannitol

Magnesium stearate

### Ac-Di-Sol

### Plasdone K29/32

Silicon colloidal

### Crospovidone

Stearic acid

### Isomalt

### Opadry II 85F19000 Clear

Aliquots of racemic isometheptene mucate and each excipient were weighed in a ratio of 1:1 (unless specified otherwise) and ground in an agate mortar. The homogeneous mixtures then were analyzed.

### Differential Scanning Calorimetry (DSC)

The DSC heating curves were obtained with a TA 821 DSC Mettler instrument under the following conditions:

Heating rate: 10° C./min
Ambient: Nitrogen 30 mL/min
Sample holder: normal open aluminum pan
Temperature range: from 25° C. to 250° C.
Instrument calibration: Indium sample purity 99.999%

### X-Ray Powder Diffraction (XRPD)

X-ray powder diffraction (XRPD) tests were performed with the ULTIMA IV instrument (Rigaku), laying the sample on a static sample holder. The X-ray focusing slit has a variable width, interlocked with the q value. The X-ray tube has a copper target, with a current intensity of 40 mA and a voltage of 40 kV. Radiation was generated by the Cockcroft-Walton method, and was constituted by K₊₁ (1.540562 Å) and K_{α2} (1.544398 Å). The analytical conditions were:

Fixed Time; sampling width 0.02 deg, scanning rate 1.3 s/step, 2 q range 3.35 deg and sample holder; amorphous glass equiangular 9200/2G, 0.2 mm deep. The sample was pressed with a glass plate.

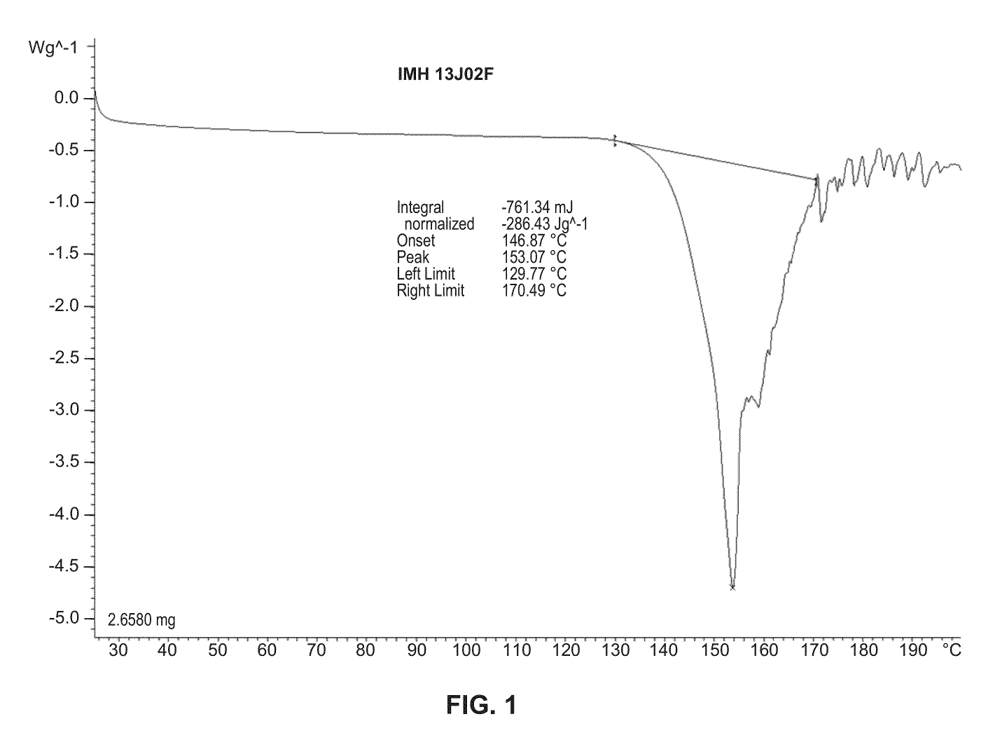

With racemic isometheptene mucate alone, melting with decomposition was detected between 130° C. and 170° C. (onset at 146.9° C., ΔH=−286.4 J/g) (FIG. 1).

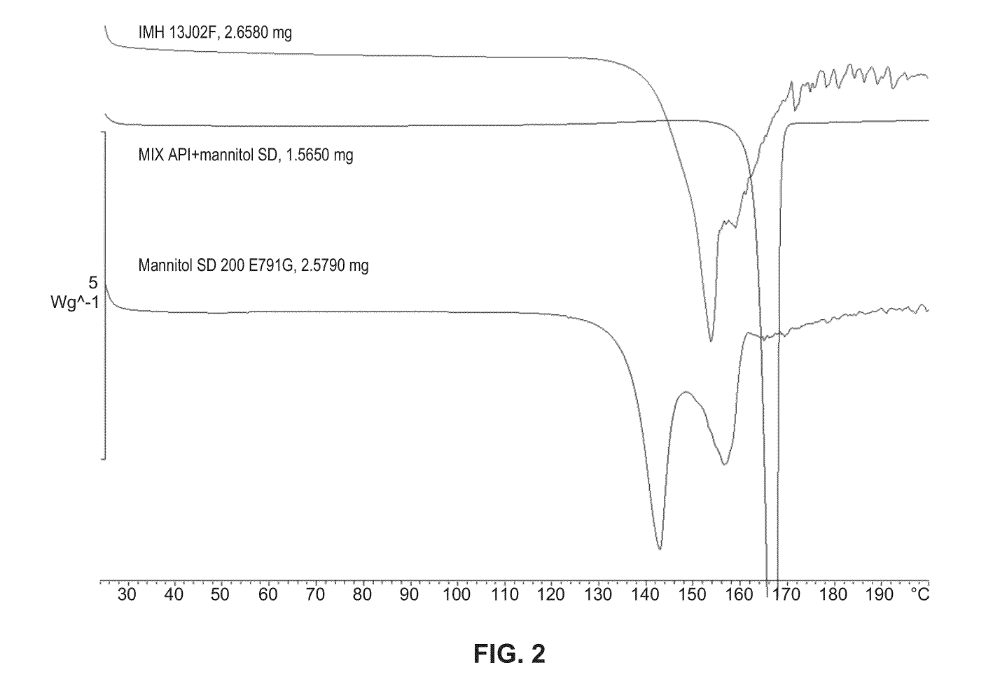

In a 1:1 mixture of racemic isometheptene mucate and mannitol SD₂₀₀, the endothermic transitions were recorded in the range of 126° C. to 162° C. (onset at 136.4° C., ΔH=−249.82 J/g) (FIG. 2). Physical interaction was observed.

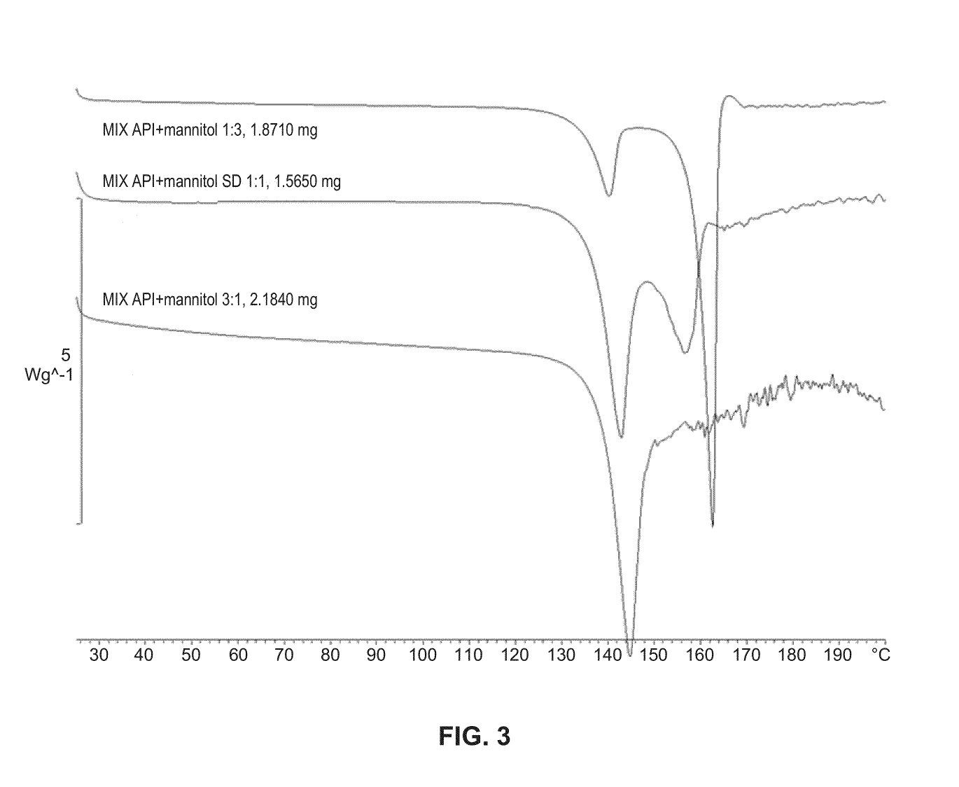

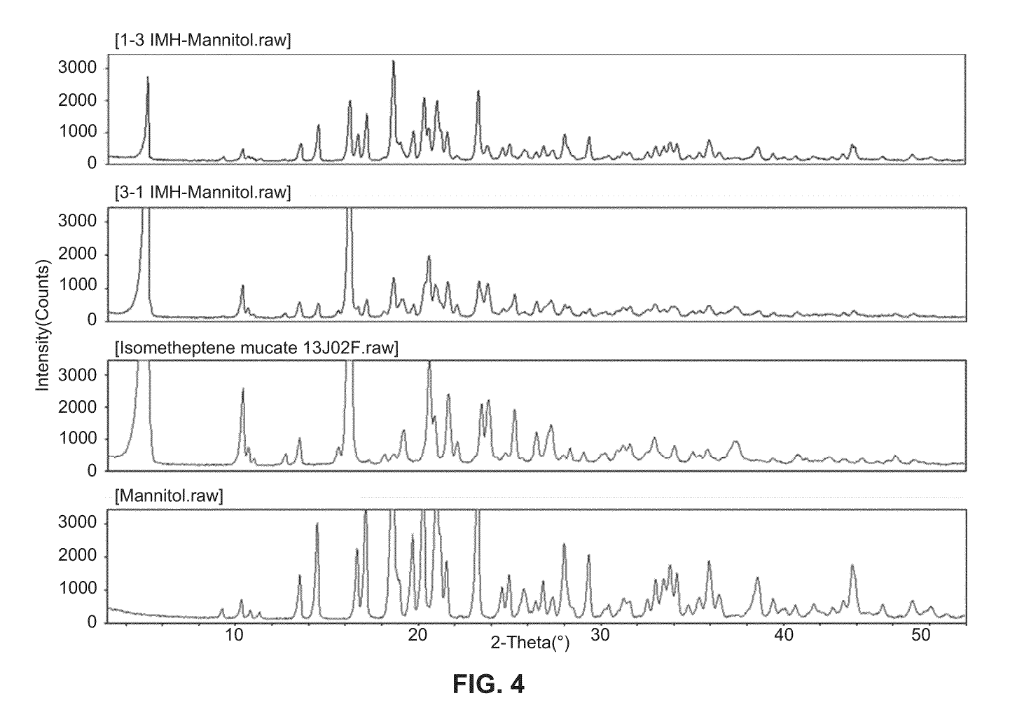

In a 3:1 mixture of racemic isometheptene mucate and mannitol SD₂₀₀, only the melting with decomposition of one single entity was observed (FIG. 3). The mixture of 1:3 racemic isometheptene mucate and mannitol SD₂₀₀ showed a transition peak at 136° C. and the melting point of predominantly mannitol. XRPD patterns of a 3:1 mixture of racemic isometheptene mucate and mannitol SD₂₀₀ and a mixture of 1:3 racemic isometheptene mucate and mannitol SD₂₀₀ showed the whole contribution of both isometheptene mucate and mannitol SD₂₀₀, a sign that only a physical interaction occurs (FIG. 4).

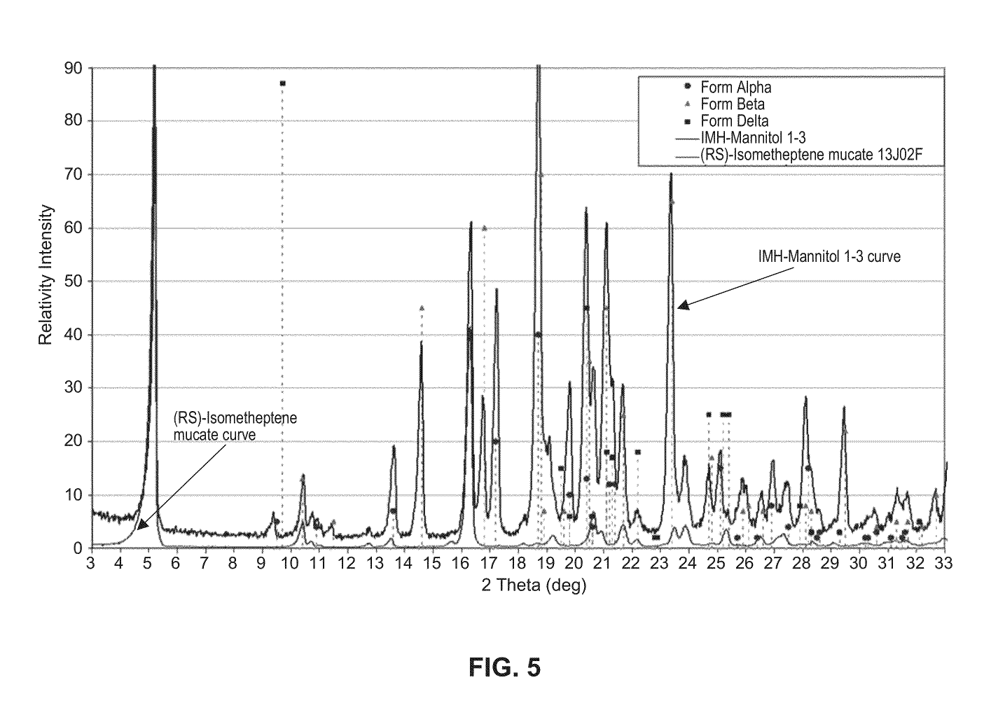

XRPD of a mixture of 1:3 racemic isometheptene mucate and mannitol SD₂₀₀ showed the presence of a mixture of mannitol with mainly β (starting material) and traces of α (FIG. 5).

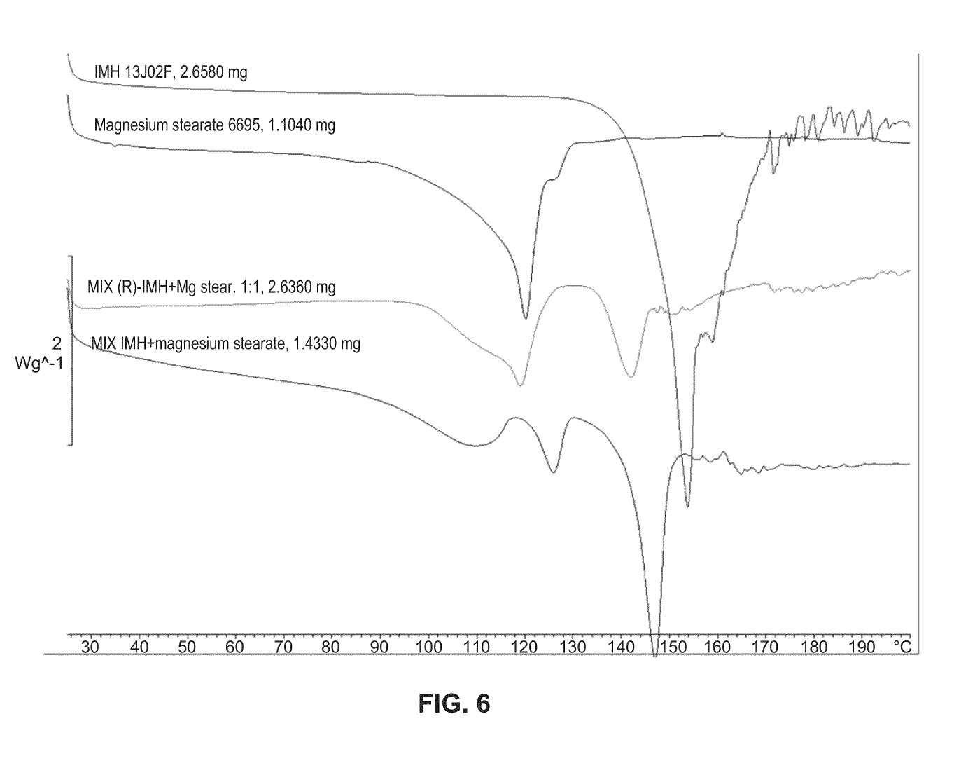

In a 1:1 mixture of racemic isometheptene mucate and magnesium stearate, a small physical interaction was observed between racemic isometheptene mucate and excipient, due to the excipient melting, that anticipates the racemic isometheptene mucate melting. The excipient transition is observed between 83-131° C., while the racemic isometheptene mucate melting peak was recorded between 133-153° C. (onset at 142° C., ΔH=−68.1 J/g) (FIG. 6).

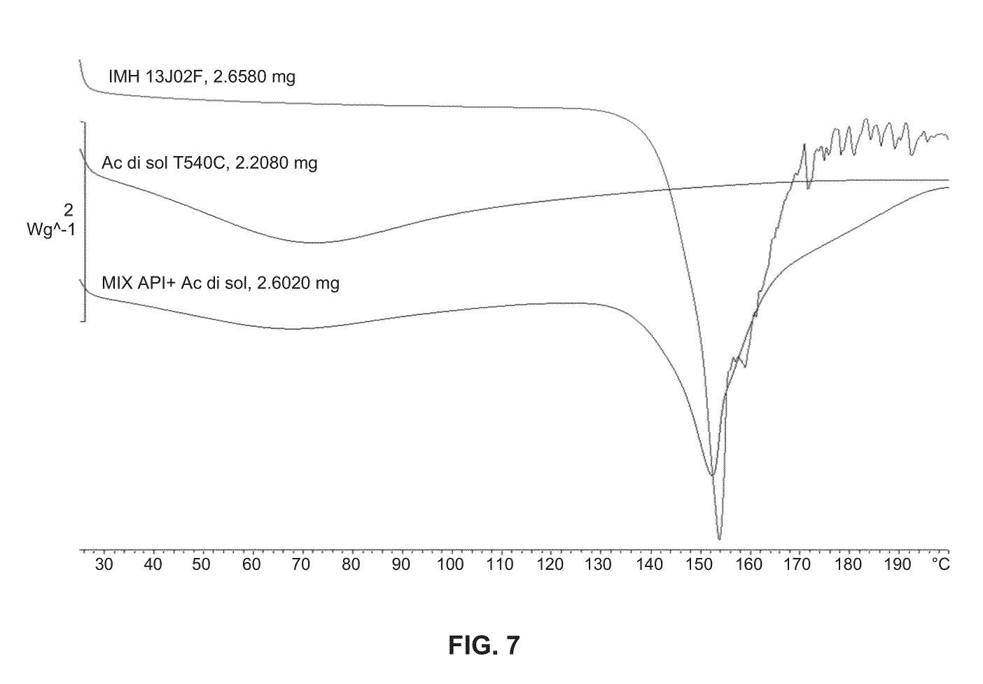

In a mixture of 1:1 racemic isometheptene mucate and Ac-Di-Sol, the release of imbibtion water from the disintegrant Ac-Di-Sol was recorded between 30-90° C., followed by the melting of racemic isometheptene mucate from 125° C. (onset at 142.1° C.) (FIG. 7). No interaction was detected. Ac-Di-Sol is a superdisintegrant (FMS BioPolymer).

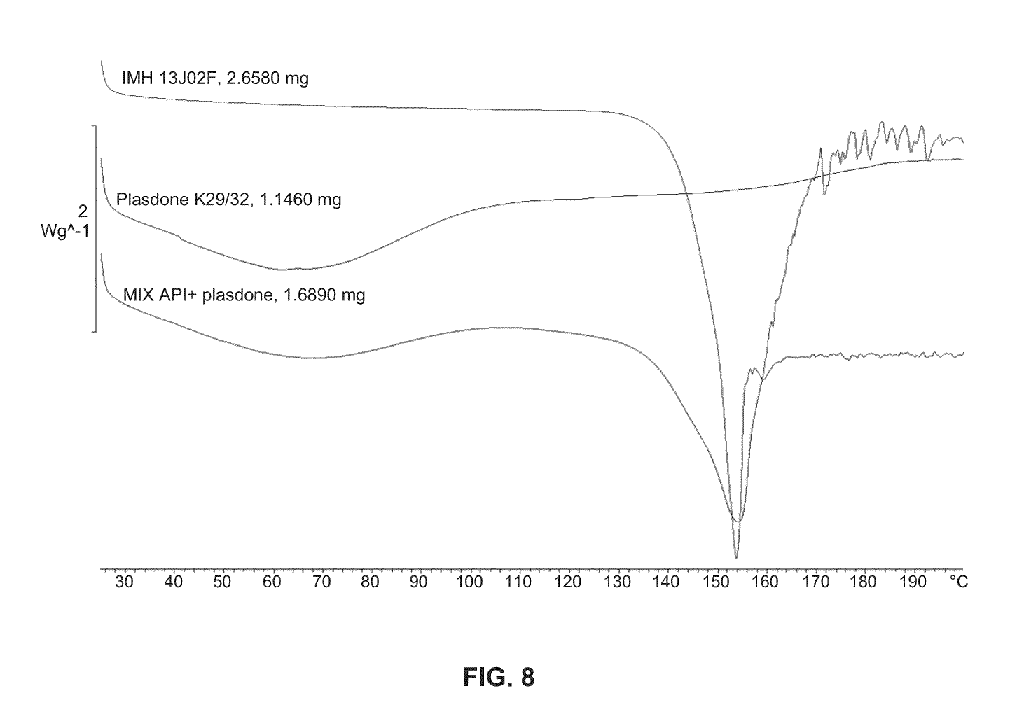

In a 1:1 mixture of racemic isometheptene mucate and plasdone, the release of imbibition water was recorded between 30-100° C., followed by the melting/decomposition of racemic isometheptene mucate from 105° C. (onset at 139.7° C.) (FIG. 8). No interaction was detected.

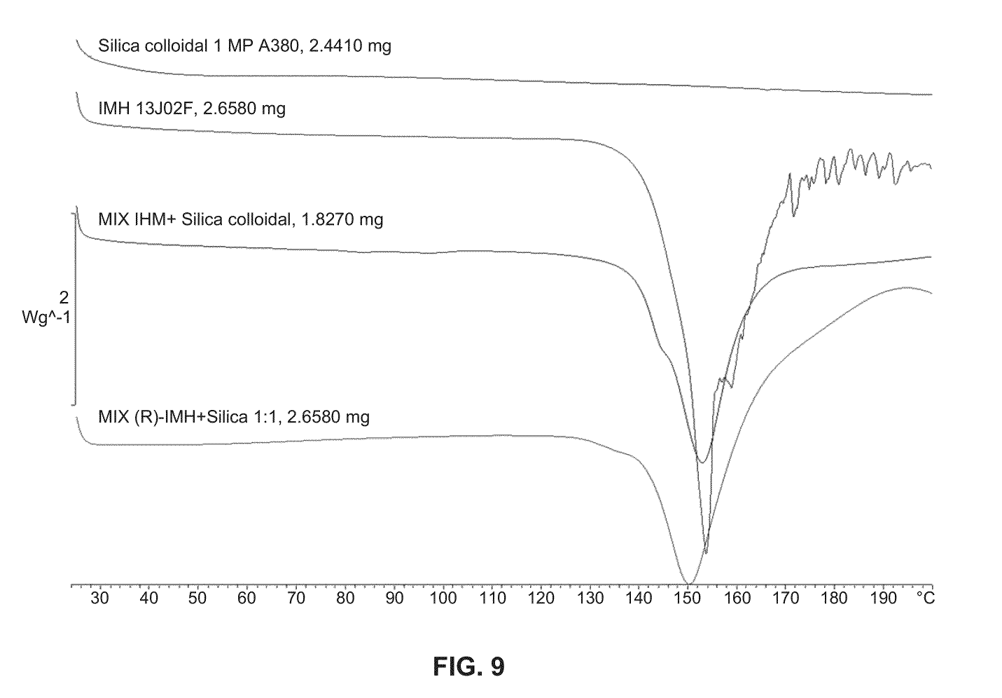

In a 1:1 mixture of racemic isometheptene mucate and silicon (colloidal), the racemic isometheptene mucate melting/decomposition peak was recorded between 130-174° C. (onset at 142.3° C., ΔH=−157.1 J/g) (FIG. 9). No interaction was detected, only a lowering of the degree of crystallinity of racemic isometheptene mucate.

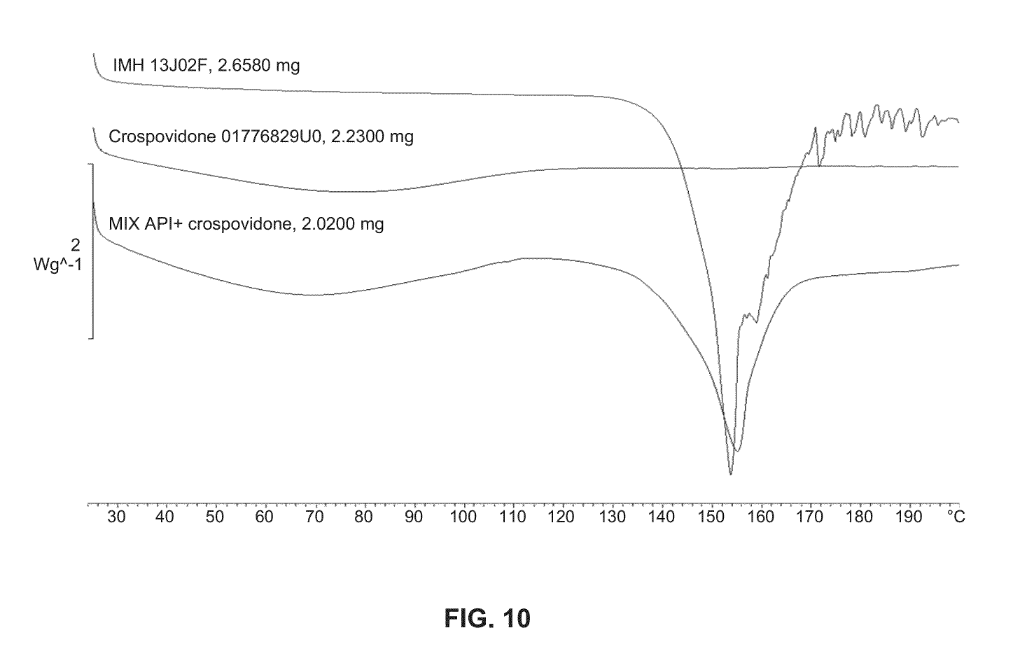

In a 1:1 mixture of racemic isometheptene mucate and crospovidone, the release of imbibition water was recorded between 30-100° C., followed by the melting/decomposition of racemic isometheptene mucate from 117° C. (onset at 143.1° C.) (FIG. 10). No interaction was detected.

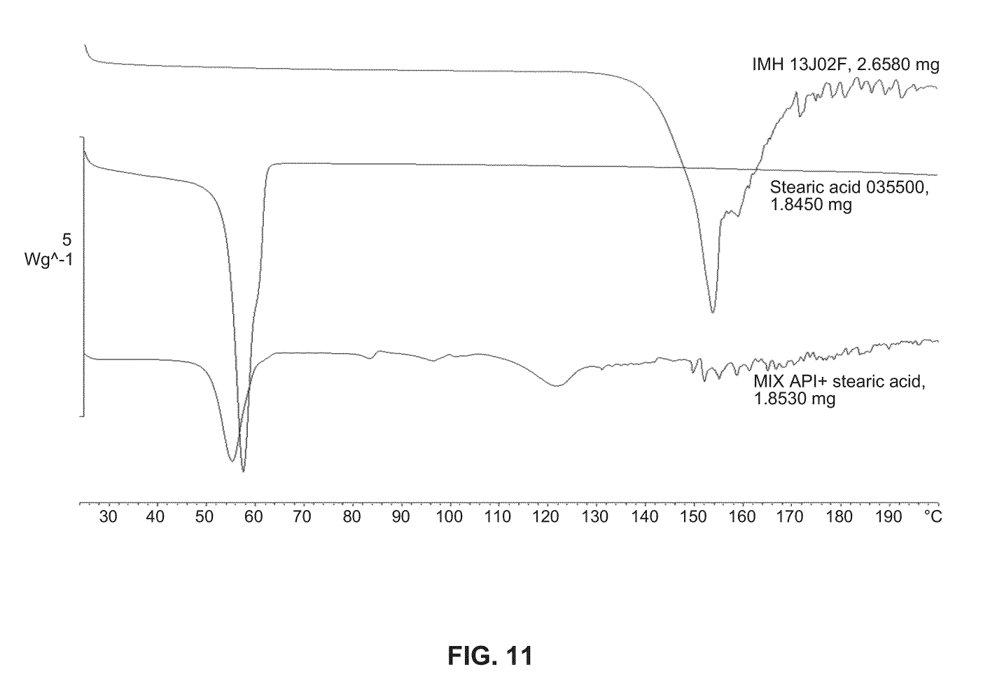

In a 1:1 mixture of racemic isometheptene mucate and stearic acid, a physical interaction was observed due to excipient melting that anticipates racemic isometheptene mucate melting. The excipient transition was observed between 43° C. and 67° C. (onset at 50.7° C., ΔH=−64.0 J/g) (FIG. 11). The racemic isometheptene mucate melting peak was anticipated between 109-130° C. (onset at 113.5° C., ΔH=−18.4 J/g). This was due to partial solubilization of racemic isometheptene mucate by the melted stearic acid.

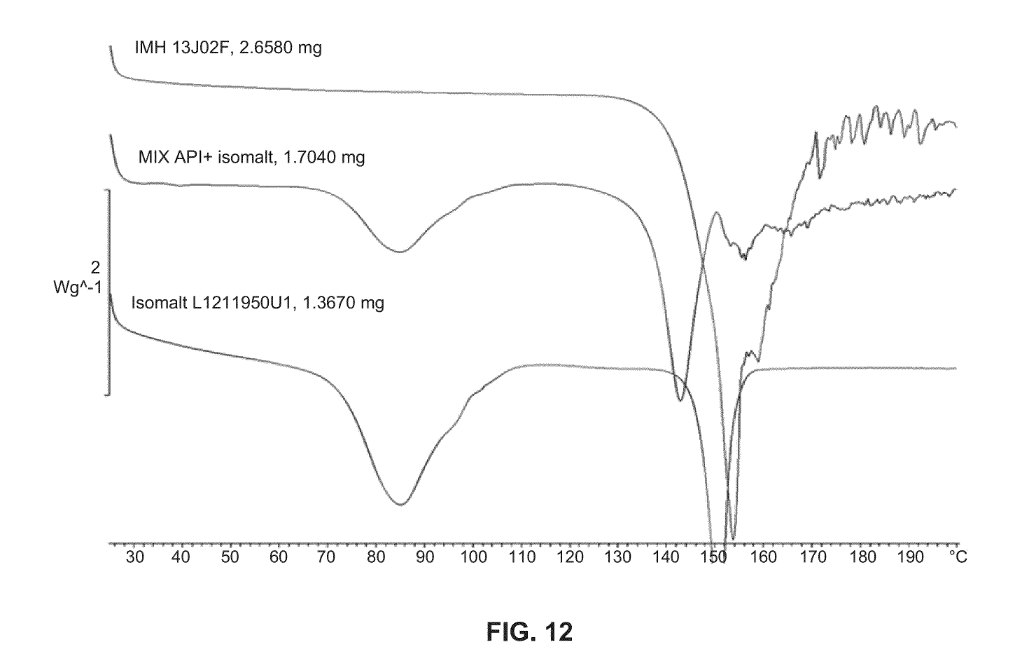

In a 1:1 mixture of racemic isometheptene mucate and isomalt, the excipient transition was observed between 63-104° C. (onset at 72.8° C., ΔH=−43.5 J/g), while the racemic isometheptene mucate melting peak was recorded between 124-151° C. (onset at 136.7° C., ΔH=−84.1 J/g) (FIG. 12). No interaction reported. Isomalt is a sugar substituted with sugar-like physical properties, but very low in calories.

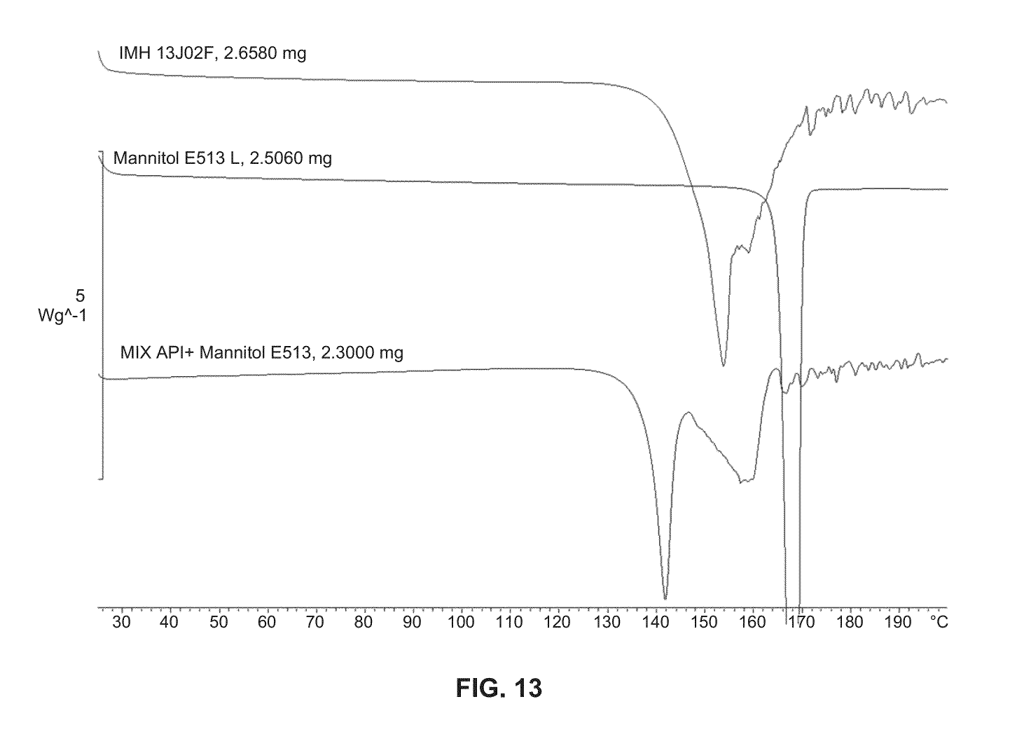

In a 1:1 mixture of racemic isometheptene mucate and mannitol, the endothermic transitions were recorded in the range of 125° C. to 165° C. (onset at 137.7° C., ΔH=−227.7 J/g) (FIG. 13). Physical interaction was observed and mannitol had the same behavior as mannitol SD₂₀₀.

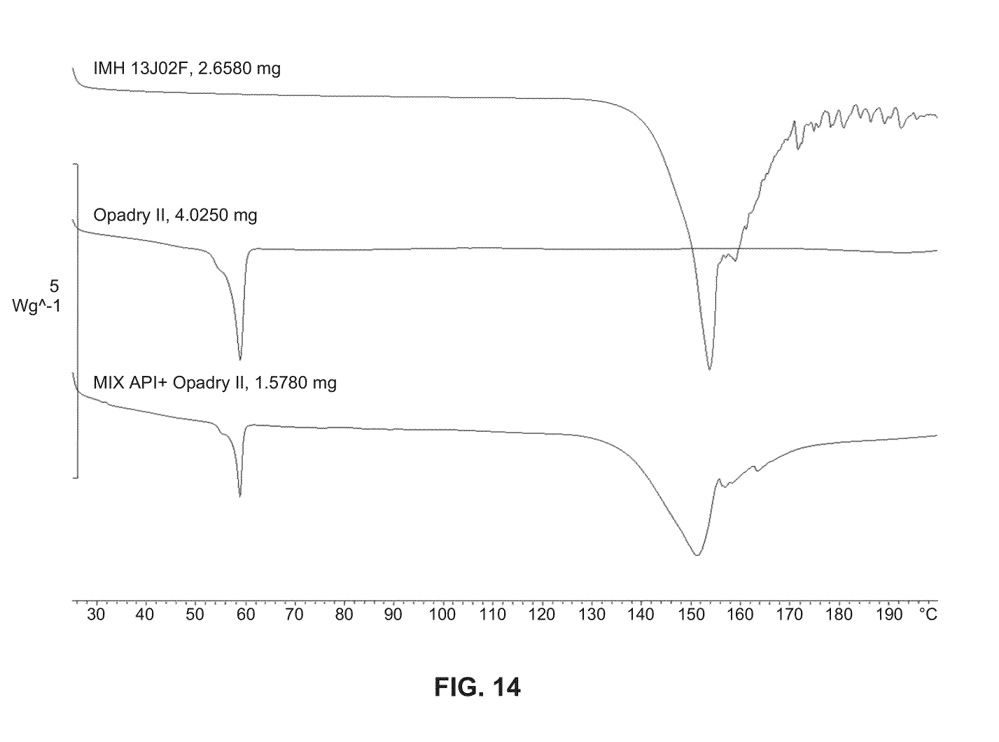

In a 1:1 mixture of racemic isometheptene mucate and Opadry II Clear, the PEG transitions were visible between 51-63° C. followed b the racemic isometheptene mucate peak in the range of 129-156° C. (onset at 139.1° C., ΔH=−74.8 J/g) (FIG. 14). This could be due to partial solubilization of the racemic isometheptene mucate into the melted excipient.

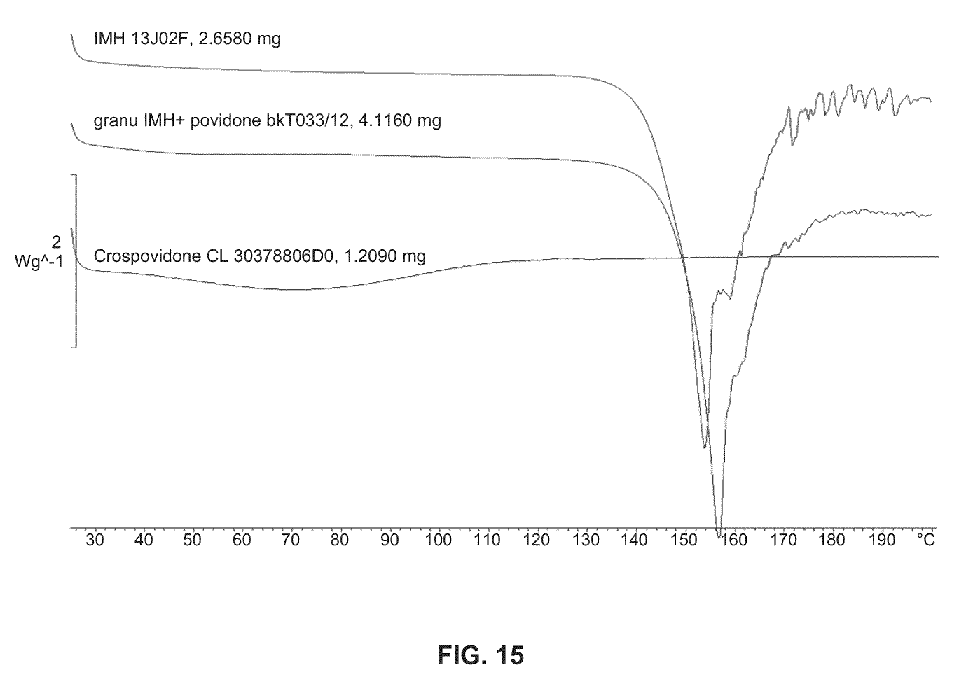

In a 1:1 mixture of granulated racemic isometheptene mucate and povidone, the melting with decomposition of racemic isometheptene mucate was recorded between 128-176° C. (onset at 150.5° C., ΔH=−233.8 J/g) (FIG. 15). No interaction reported.

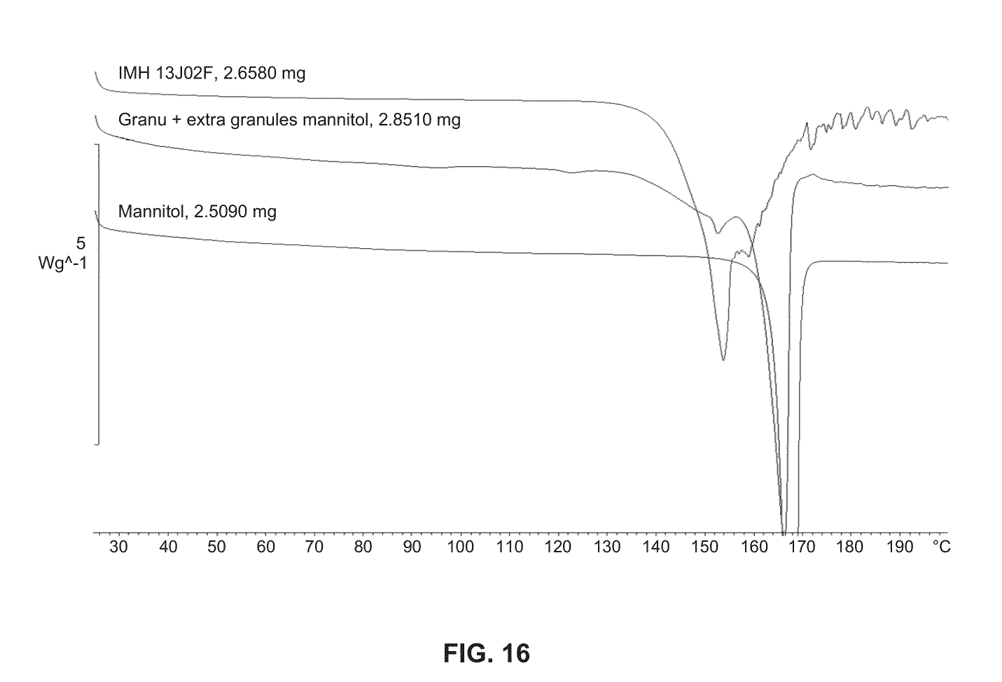

In a 1:1 mixture of granulated racemic isometheptene mucate and extra granules of mannitol, the magnesium stearate transitions were recorded between 80-103° C. (onset at 83.9° C., ΔH=−2.2 J/g) and between 116-128° C. (onset at 119.3° C., ΔH=−1.1 J/g), followed by the melting of racemic isometheptene mucate and mannitol together between 129-172° C. (onset at 160.2° C., ΔH=−221.1 J/g) (FIG. 16). The racemic isometheptene mucate peak is well visible at 150° C. No remarkable interaction reported.

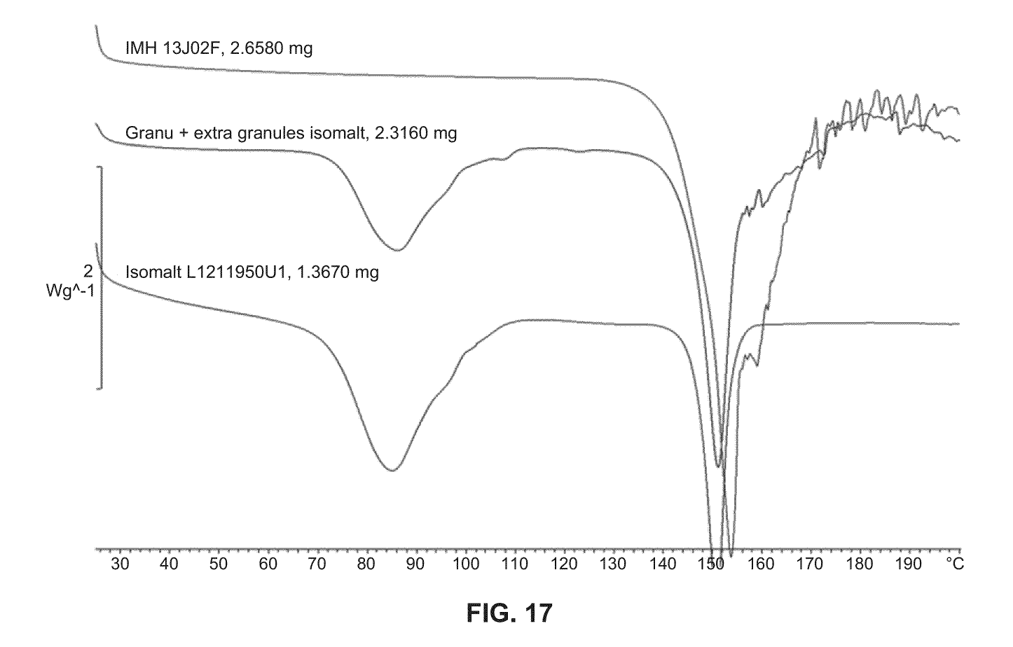

In a 1:1 mixture of granulated racemic isometheptene mucate and extra granules of isomalt, the isomalt transition, due to water release, was observed between 66-100° C. (onset at 74.3° C., ΔH=−56.7 J/g), followed by magnesium stearate transitions between 103-112° C. (onset at 105.1° C., ΔH=−1 J/g) and between 117-127° C. (onset at 119.6° C., ΔH=−0.5 J/g). The melting of racemic isometheptene mucate and isomalt occurred together between 134-157° C. (onset at 145.5° C., ΔH=−84.7 J/g) (FIG. 17). No remarkable interaction observed.

In summary, different types of interactions were observed among the excipients and the racemic isometheptene mucate. A physical interaction was observed both with stearic acid and with magnesium stearate (in a ratio of 1:1), probably due to the partial racemic isometheptene mucate solubilization after excipient melting. Since in the final formulation these two excipients are present in very low amounts, the interaction could be considered unimportant or inconclusive. Lowering the degree of crystallinity of racemic isometheptene mucate was observed with colloidal silicon. A physical interaction was observed with mannitol, both by means of thermal analysis and XRPD. No formation of the adduct was observed at different racemic isometheptene mucate:mannitol ratios (e.g., 1:3 and 3:1). All final granulate compositions with extra granules of excipient show no interaction with racemic isometheptene mucate. The addition of extra granules of isomalt or mannitol does not create interactions racemic isometheptene mucate. Table 1 summarizes the observations of the various excipients with racemic isometheptene mucate API.

TABLE 1
Excipient reactions with API (racemic isometheptene mucate)

| Excipient | Mixture 1:1 |
|---|---|
| Mannitol SD₂₀₀ | Physical interaction (complex) |
| Magnesium Stearate | YES |
| | (physical interaction after melting) |
| Ac-di-Sol | NO |
| Plasdone K29/32 | NO |
| Silicon colloidal | No interaction |
| | (reduction of crystallinity degree) |
| Crospovidone | NO |
| Stearic acid | YES |
| | (physical interaction after melting) |
| Isomalt | NO |
| Mannitol | Physical interaction (complex) |
| Opadry II | NO |
| Granulated IMH + Povidone | NO |
| Mix granulated + extra | NO |
| granules of mannitol | |
| Mix granulated + extra | NO |
| granules of isomalt | |

### Example 2

The compatibility of mannitol with racemic isometheptene mucate was investigated by differential scanning calorimetry (DSC), and the resulting interactions were assessed. In particular, the formation of a eutectic between the mannitol and the racemic isometheptene mucate during mixing improved the cohesion between the particles and provided better physical bonding between the racemic isometheptene mucate active pharmaceutical ingredient (API) and the mannitol excipient.

The interaction between racemic isometheptene mucate and mannitol SD₂₀₀ is an invariant physical interaction because it is in thermal equilibrium in which the two components are well mixed and stabilized. Physically, this means that the melted eutectic, solid eutectic, and solid mannitol all coexist at the same time and are in chemical equilibrium. The resulting solid macrostructure from the eutectic reaction depends on a few factors, including that the two solid solutions nucleate and grow together during a mechanical mixture.

Because mannitol is a common excipient in solid drug formulations, it was examined for compatibility with racemic isometheptene mucate and investigated using DSC and the interactions occurring were assessed. Surprisingly, the formation of a eutectic during mechanical mixing was discovered. To confirm the formation of a eutectic and to characterize its physical properties, several binary mixtures at different ratios of racemic isometheptene mucate and excipient were prepared and analyzed by DSC and by XRPD. The eutectic formation improved the cohesion between the racemic isometheptene mucate and excipient particles and assured better physical linking between the two.

In order to confirm the eutectic formation and to characterize its physical properties, several binary mixtures at different ratios of racemic isometheptene mucate-excipient were prepared and analyzed by DSC and by X-ray powder diffraction (XRPD). The mixtures were obtained by gently milling in agate mortar of micronized racemic isometheptene mucate and mannitol, in order to obtain homogeneous distribution of the particles. For each DSC heating curve, the onset temperature and the enthalpy were evaluated both for the eutectic contribute and for the excess of component. The recorded values were plotted and a phase diagram between the two components was obtained with a characteristic profile of phase diagrams of eutectic mixtures.

Mixtures also were investigated by XRPD and compared with the patterns of pure components. These analyses were carried out to confirm that the eutectic compound is only a physical interaction between the two products and not a formation of a new entity with different chemical properties.

Aliquots of racemic isometheptene mucate API and mannitol were weighed in the ratios described below and ground in an agate mortar, and the homogeneous mixtures subsequently analyzed.

| Mixture | API amount (%) |
|---|---|
| 1 | 0 |
| 2 | 25 |
| 3 | 33 |
| 4 | 40 |
| 5 | 50 |
| 6 | 60 |
| 7 | 67 |
| 8 | 75 |
| 9 | 90 |
| 10 | 100 |

### Differential Scanning Calorimetry (DSC)

DSC heating curves were obtained using a TA 821 DSC Mettler instrument under the following conditions:

| | |
|---|---|
| Heating rate | 10° C./min |
| Ambient | Nitrogen 30 mL/min |
| Sample order | Normal open aluminum pan |
| Temperature range | From 25° C. to 250° C. |
| Instrument calibration | Indium sample purity 99.999% |

### X-Ray Powder Diffraction (XRPD)

X-ray powder diffraction (XRPD) tests were performed with a ULTIMA IV (Rigaku) instrument, laying the sample on a static sample holder. The X-ray focusing slit had a variable width, interlocked with the 0 value. The X-ray tube had a copper target, with a current intensity of 40 mA and a voltage of 50 kV. The radiation generated by the Cockcroft-Walton method is constituted by K_{α1} (1.540562 Å) and K_{α2} (1.544398 Å). The analytical conditions were as follows:

Fixed Time: Sampling width, 0.02 deg; Scanning rate, 1.0 s/step
2θ range: 3/50 deg.
Sample holder: amorphous glass—equiangular 9200/2G, 0.2 mm deep. The sample was pressed with a glass plate.

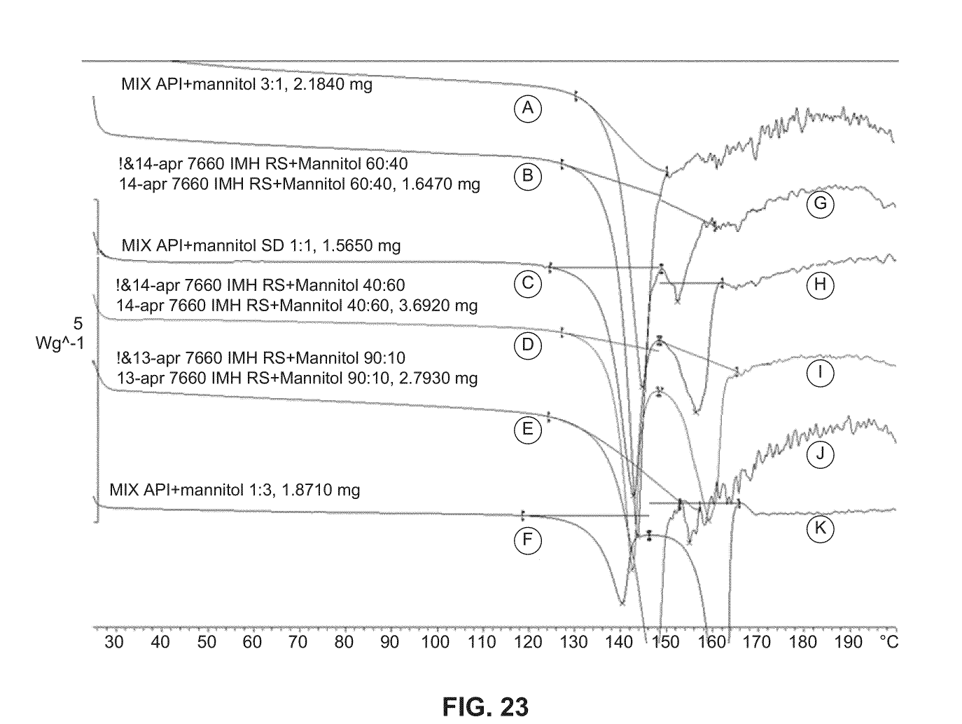

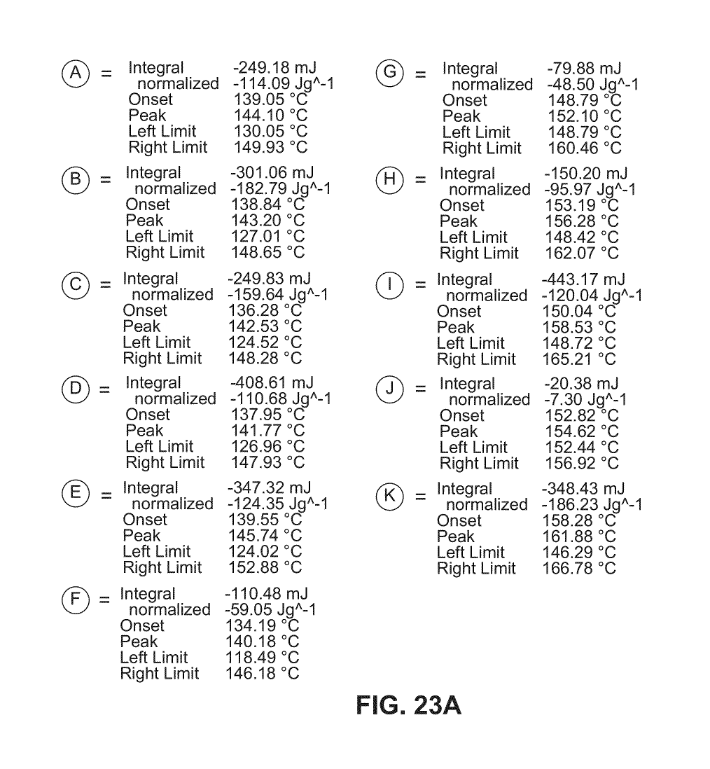

Pure components of racemic isometheptene mucate and mannitol, as well as mixtures of the two, were analyzed with DSC. FIG. 1 depicts the melting curve with 100% racemic isometheptene mucate. Melting with decomposition was detected starting from 125° C. (onset at 146° C.). FIG. 2 depicts the melting curve with 100% mannitol. Melting was detected between 151° C. and 172° C. (onset at 163.9° C., ΔH=−241.1 J/g). FIGS. 23 and 23A depict the various mixtures. Table 2 summarizes the data.

TABLE 2
Summary of DSC data

| | | | T onset | | ΔH | | |
|---|---|---|---|---|---|---|---|
| | % | T onset | 2nd | ΔH | 2nd | ΔH | |
| | Man- | eutectic | effect | eutectic | effect | global | |
| % API | nitol | (° C.) | (° C.) | (J/g) | (J/g) | (J/g) | Plot |
| 0 | 100 | — | 163.86 | — | — | 241.07 | FIG. 2 |
| 33 | 67 | 134.19 | 158.28 | 59.05 | 186.23 | — | FIG. 23 |
| 40 | 60 | 137.95 | 150.04 | 110.68 | 120.04 | — | FIG. 23 |
| 50 | 50 | 136.28 | 153.19 | 159.64 | 95.97 | — | FIG. 23 |
| 60 | 40 | 138.84 | 148.79 | 182.79 | 48.50 | — | FIG. 23 |
| 67 | 33 | 139.05 | — | 114.09 | — | — | FIG. 23 |
| 90 | 10 | 139.55 | 152.82 | 124.35 | 7.30 | — | FIG. 23 |
| 100 | 0 | — | 146.87 | — | — | 286.43 | FIG. 1 |

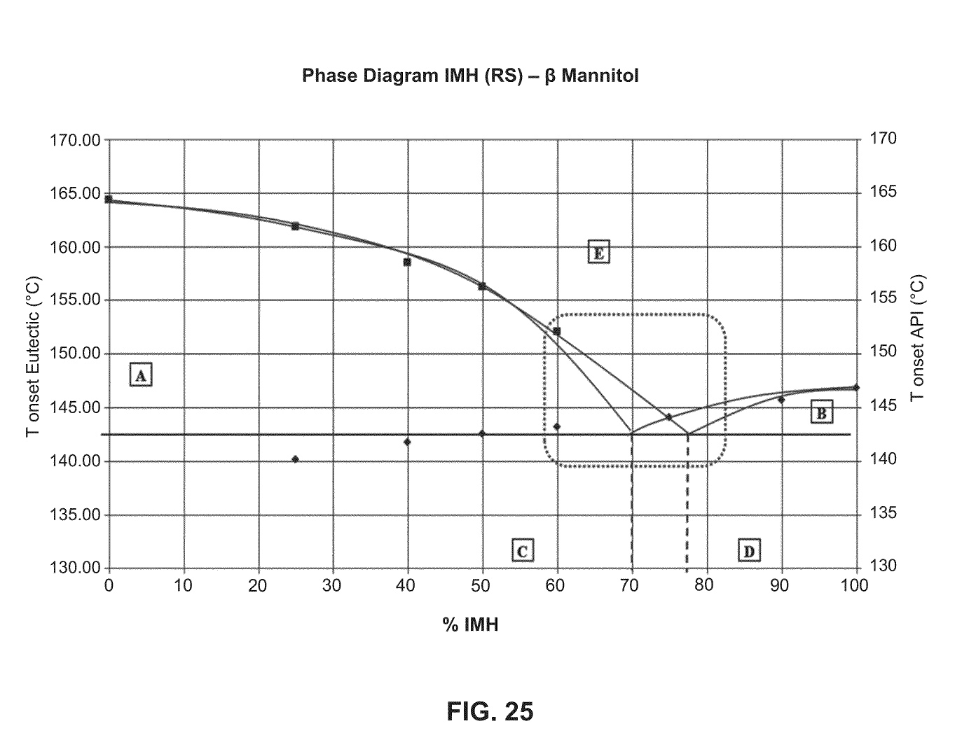

The above results demonstrated that the eutectic composition formed at approximately 77% racemic isometheptene mucate (API) and 23% mannitol. Under approximately 70%, two distinct melting peaks were observed from the melting of the eutectic fraction and the excess of the individual components. FIG. 25 shows a phase diagram depicting the onset melting temperatures of the eutectic fraction and the excess components, plotted as function of racemic isometheptene mucate percentage. Five distinct zones are present in the diagram:

Zone A: Excess of mannitol (liquid eutectic+solid mannitol)
Zone B: Excess of racemic isometheptene mucate (liquid eutectic+solid racemic isometheptene mucate)
Zone C: Solid eutectic with mannitol
Zone D: Solid eutectic with racemic isometheptene mucate
Zone E: Liquid phase with mannitol and racemic isometheptene mucate

In Zone A, when the percentage of racemic isometheptene mucate increased, the onset temperature of the excess of mannitol decreased while the temperature of eutectic fraction remained constant around 143° C. Above the eutectic composition, the excess of racemic isometheptene mucate led to an increase in the temperature (Zone B). In addition, there was a good correlation between mixtures and temperature. A few small deviations from the trend curve were due to an incompletely homogeneous powder mixture.

### XRPD

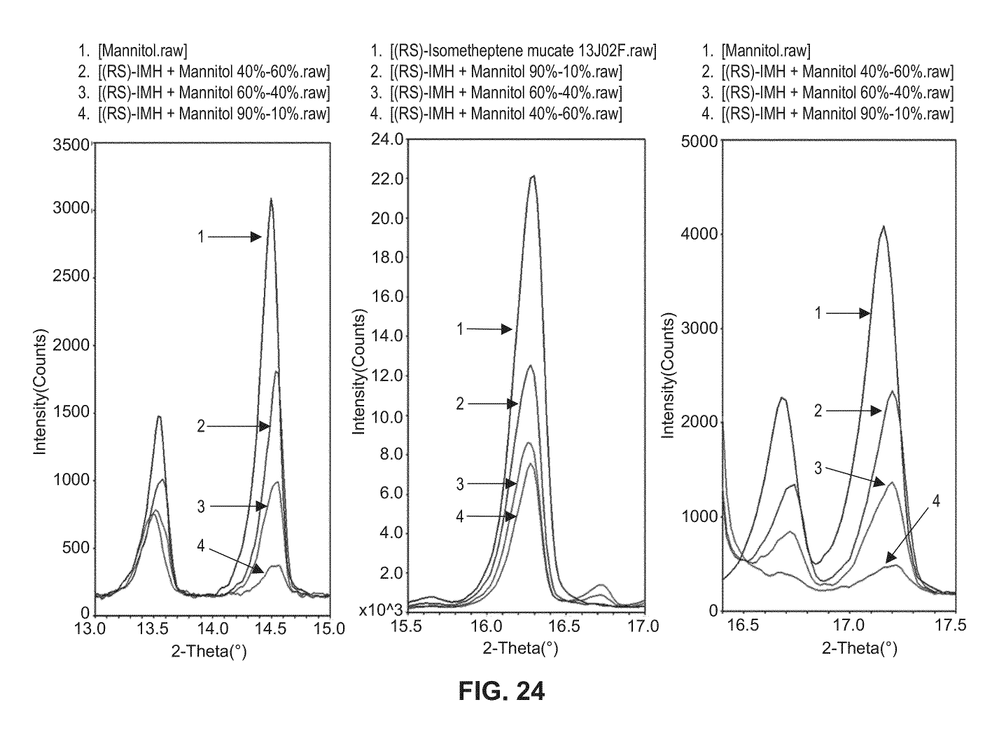

To confirm that the eutectic composition was only a physical mixture and that a new entity or adduct was not formed, the mixtures were analyzed by X-ray Powder Diffraction, where no thermal treatments were applied (pure racemic isometheptene mucate, FIG. 4; pure mannitol, FIG. 4). FIG. 5 depicts the stacking of pure mannitol, racemic isometheptene mucate, and the eutectic mixture at 33%, showing different diffraction zones where no peaks of the pure components were distinguishable and no interferences were detected. FIG. 24 shows the stacking of pure mannitol and racemic isometheptene mucate and mixtures thereof, where it was possible to point out three distinct diffraction peaks: 13.5° 2θ, 14.5° 20 and 17.2° 20.

In summary, the data show that thermal behavior of the mixtures presents two endotherms, relating to the eutectic and to the melting of the excess of the main component. Thermal entities recorded for the mixtures agreed with the percentage of racemic isometheptene mucate/mannitol ratio present in the eutectic mixture. At the eutectic composition, only one melting peak was visible. The eutectic composition was reached at about 75% racemic isometheptene mucate and 25% mannitol. The eutectic composition confirmed the molar stoichiometry (ratio between the two components: 1.0:1.0). The melting temperature of the eutectic was about 142° C. and was recorded for all the investigated mixtures. By XRPD, no adduct interaction occurred between racemic isometheptene mucate and mannitol, only a physical eutectic formation.

### Example 3

Thermal analytical techniques were used to assess the compatibility of a drug product containing racemic isometheptene mucate (API). The compatibility assessment was carried out between the racemic isometheptene mucate and a number of possible excipients in a 1:1 weight ratio. Based on the thermal events recorded for each component and for the mixtures, the analyses were carried out by investigating the peaks recorded by differential scanning calorimetry (DSC) in mixture between racemic isometheptene mucate and the excipients. Differences in thermal profiles between the single compound and the related mixture obtained after milling the products in an agate mortar were evaluated.

The following raw materials were used:

(R)-isometheptene mucate

### Mannitol SD₂₀₀

Magnesium stearate

### Plasdone K29/32

Silicon colloidal

### Crospovidone

### Isomalt

Aliquots of (R)-isometheptene mucate and each excipient were weighed in a ratio of 1:1 (unless specified otherwise) and ground in an agate mortar. The homogeneous mixtures then were analyzed.

### Differential Scanning Calorimetry (DSC)

The DSC heating curves were obtained with a TA 821 DSC Mettler instrument under the following conditions:

Heating rate: 10° C./min
Ambient: Nitrogen 30 mL/min
Sample holder: normal open aluminum pan
Temperature range: from 25° C. to 250° C.
Instrument calibration: Indium sample purity 99.999%

### X-Ray Powder Diffraction (XRPD)

X-ray powder diffraction (XRPD) tests were performed with the ULTIMA IV instrument (Rigaku), laying the sample on a static sample holder. The X-ray focusing slit has a variable width, interlocked with the q value. The X-ray tube has a copper target, with a current intensity of 40 mA and a voltage of 40 kV. Radiation was generated by the Cockcroft-Walton method, and was constituted by K_{α1} (1.540562 Å) and K_{α2} (1.544398 Å). The analytical conditions were:

Fixed Time; sampling width 0.02 deg, scanning rate 1.3 s/step, 2 q range 3.35 deg and sample holder; amorphous glass equiangular 9200/2G, 0.2 mm deep. The sample was pressed with a glass plate.

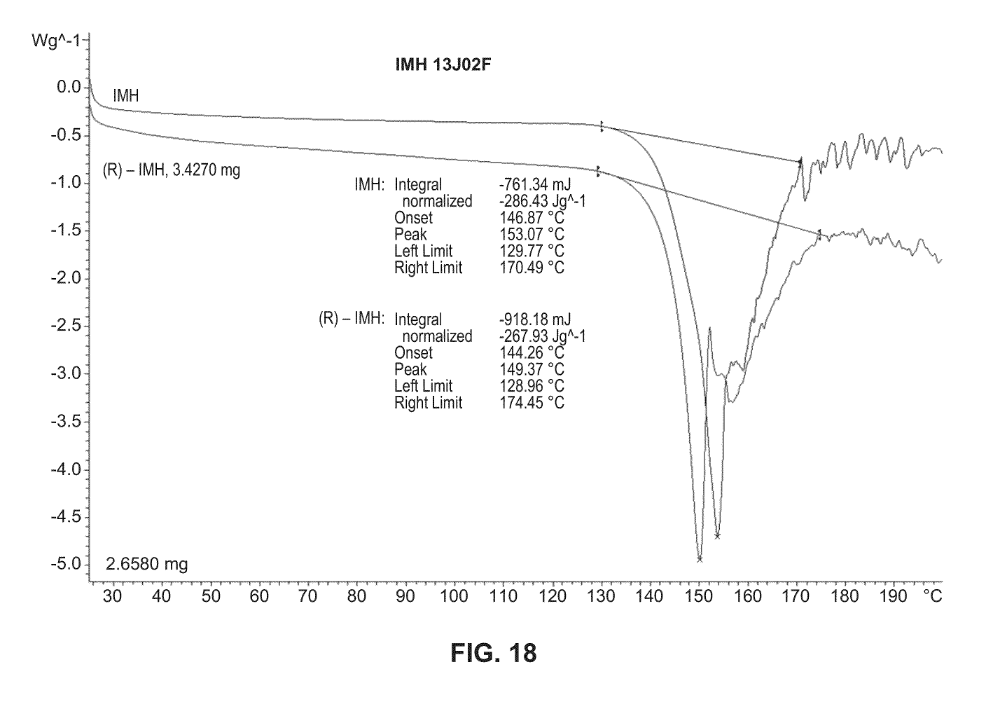

With (R)-isometheptene mucate alone, melting with decomposition was detected between 129° C. and 174° C. (onset at 144.3° C., ΔH=−267.9 J/g) (FIG. 18).

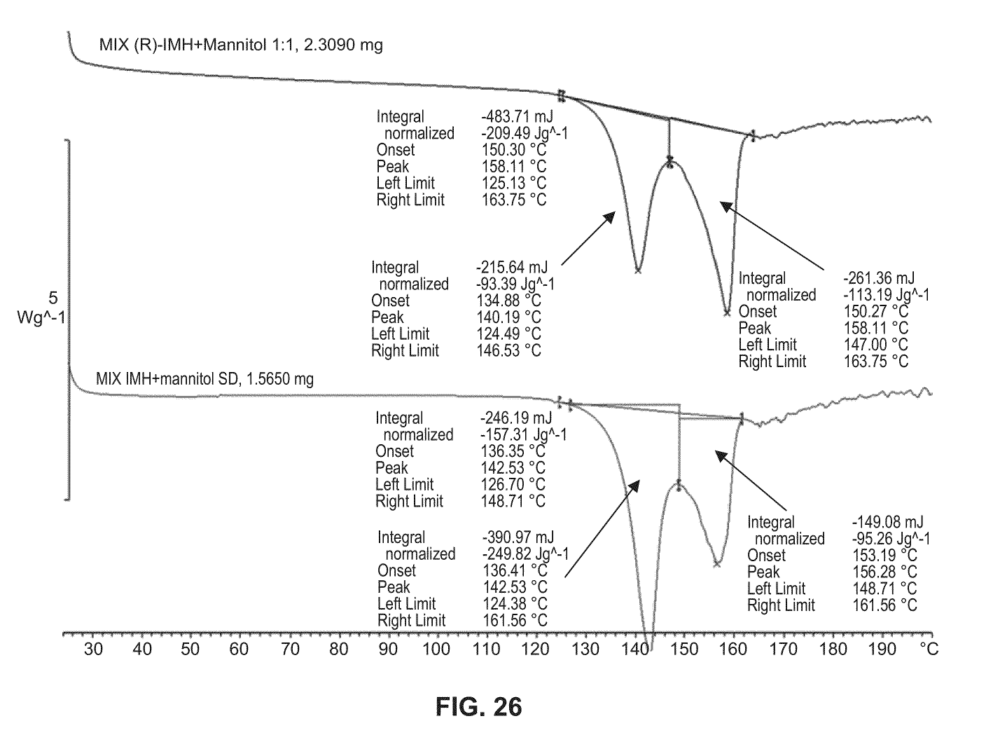

In a 1:1 mixture of (R)-isometheptene mucate and mannitol SD₂₀₀, the endothermic transitions were recorded in the range of 126° C. to 162° C. (onset at 136.9° C., ΔH=−209.49 J/g) (FIG. 26). Physical interaction was observed.

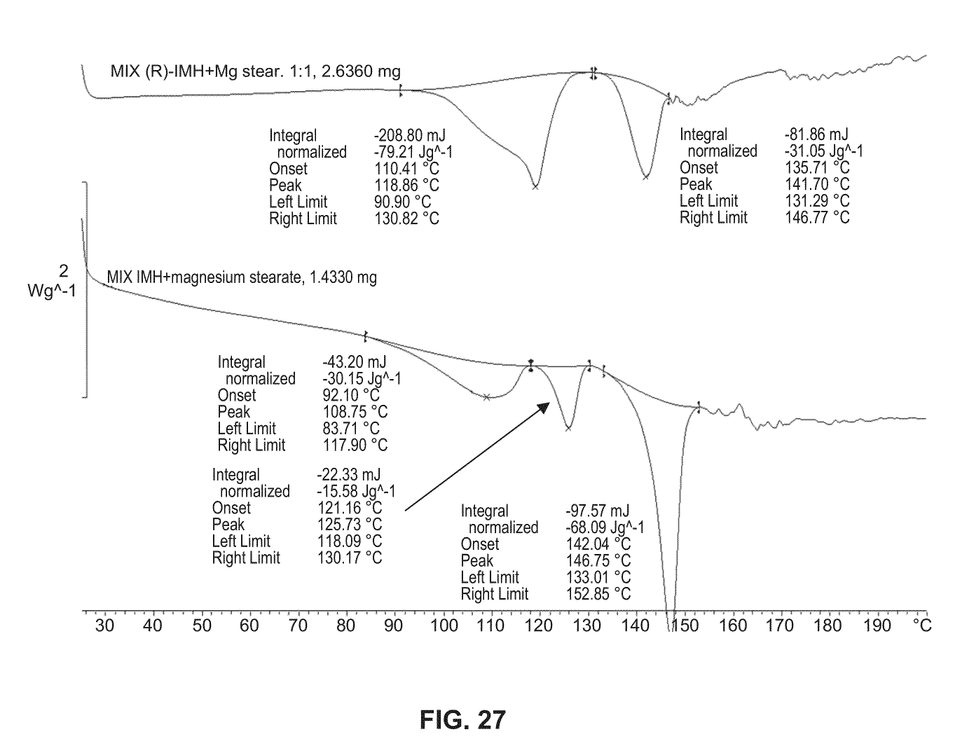

In a 1:1 mixture of (R)-isometheptene mucate and magnesium stearate, a small physical interaction was observed between (R)-isometheptene mucate and excipient, due to the excipient melting, that anticipates the (R)-isometheptene mucate melting. The excipient transition is observed between 90.9-130.8° C., while the (R)-isometheptene mucate melting peak was recorded between 131-147° C. (onset at 135.7° C., ΔH=−31.05 J/g) (FIG. 27).

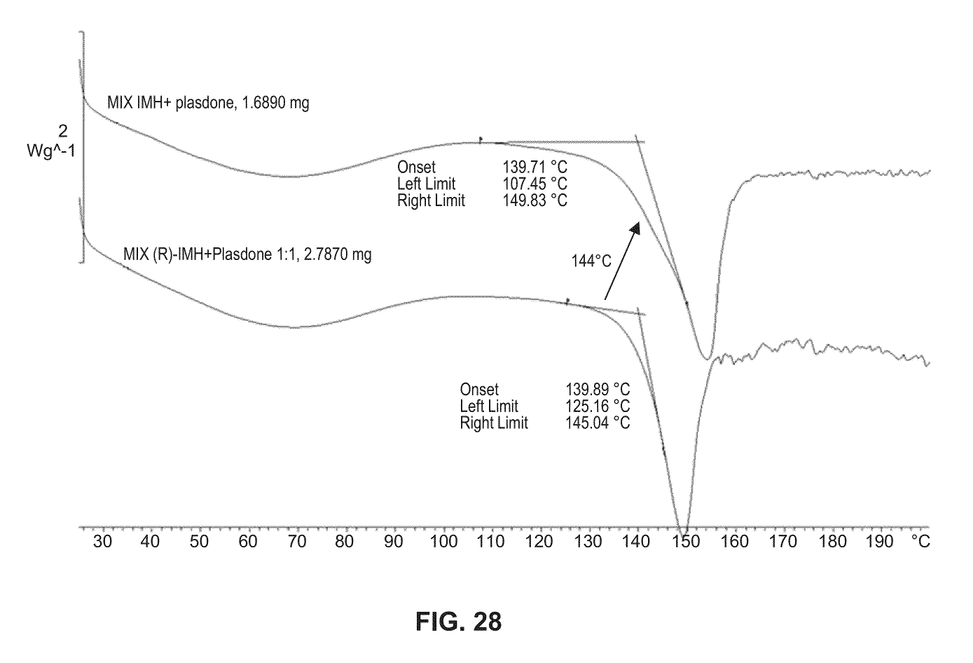

In a 1:1 mixture of (R)-isometheptene mucate and plasdone, the release of imbibition water was recorded between 30-100° C., followed by the melting/decomposition of (R)-isometheptene mucate from 107° C. (onset at 139.7° C.) (FIG. 28). No interaction was detected.

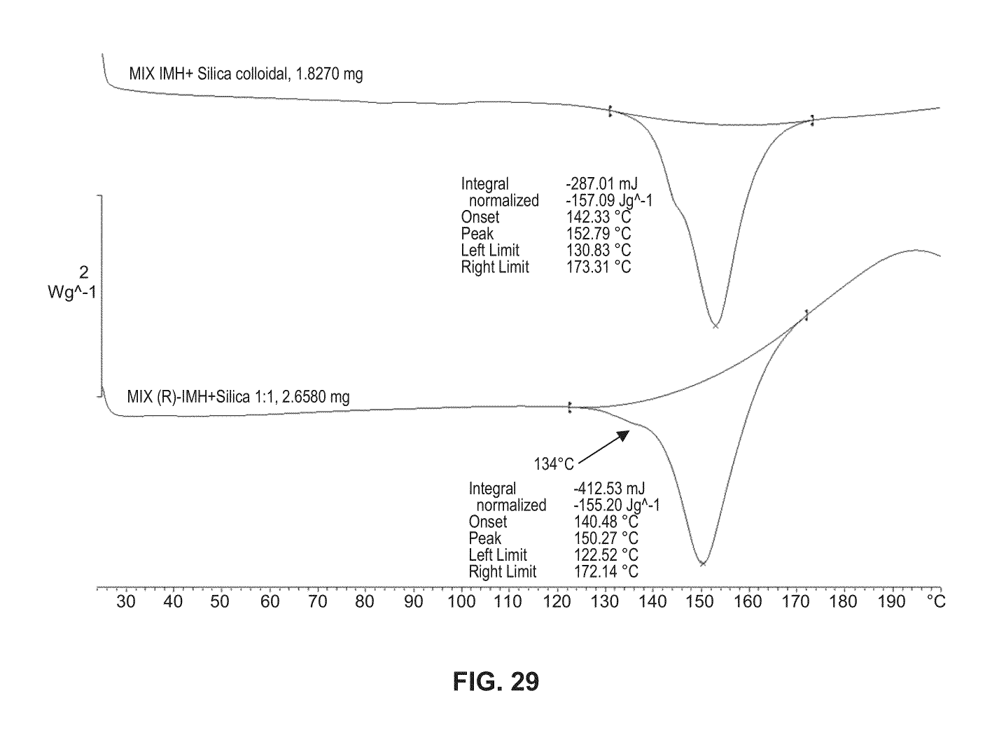

In a 1:1 mixture of (R)-isometheptene mucate and silicon (colloidal), the (R)-isometheptene mucate melting/decomposition peak was recorded between 122-172° C. (onset at 140.5° C., ΔH=−155.2 J/g) (FIG. 29). No interaction was detected, only a lowering of the degree of crystallinity of (R)-isometheptene mucate.

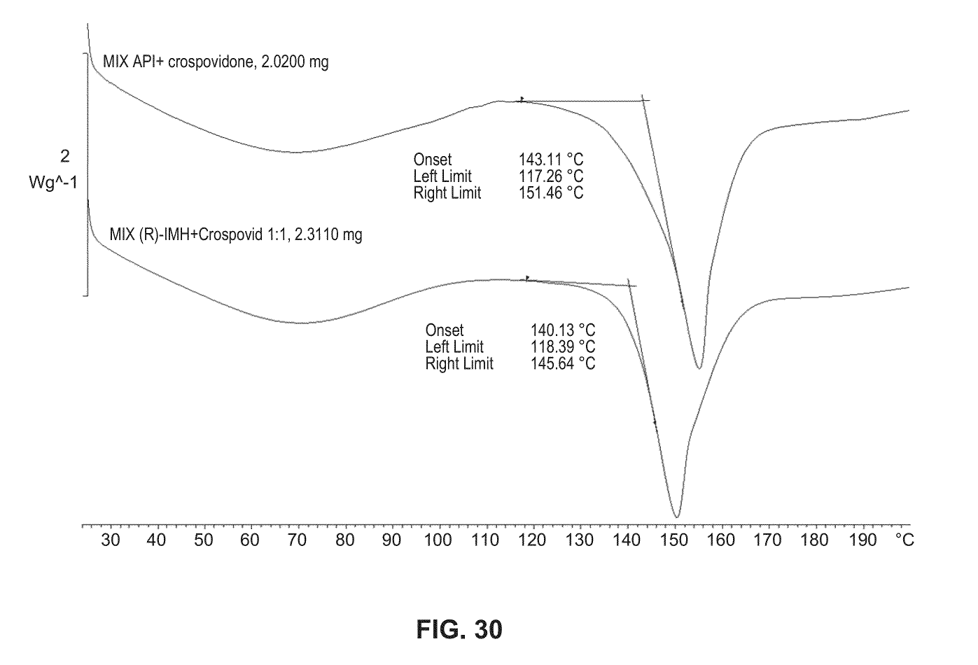

In a 1:1 mixture of (R)-isometheptene mucate and crospovidone, the release of imbibition water was recorded between 30-100° C., followed by the melting/decomposition of (R)-isometheptene mucate from 118° C. (onset at 140.1° C.) (FIG. 30). No interaction was detected.

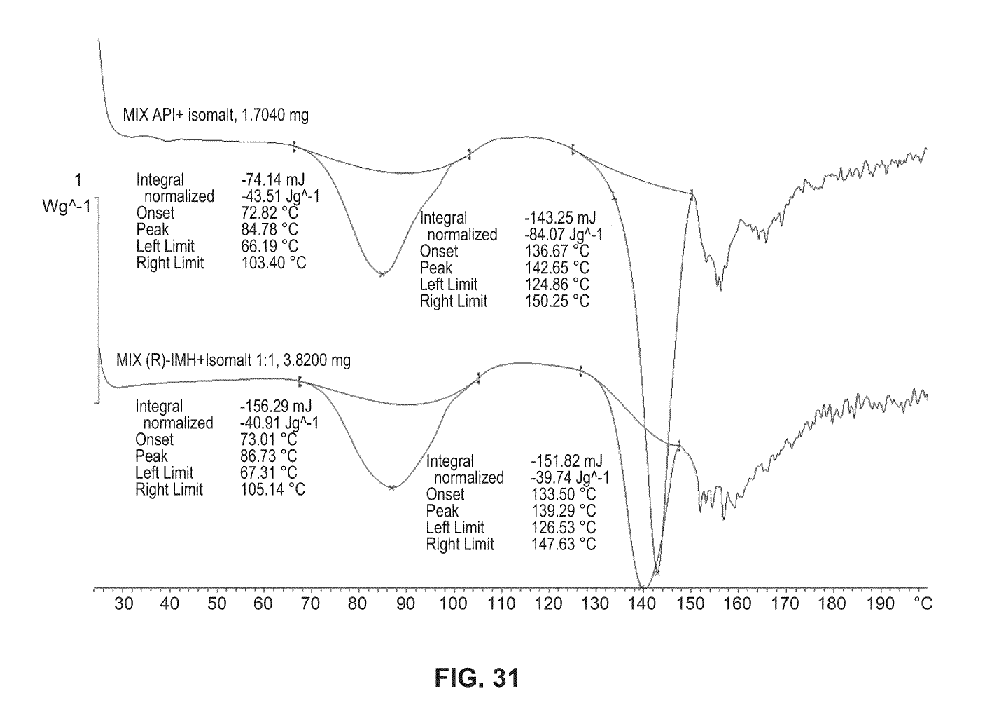

In a 1:1 mixture of (R)-isometheptene mucate and isomalt, the excipient transition was observed between 67-105° C. (onset at 73° C., ΔH=−40.9 J/g), while the (R)-isometheptene mucate melting peak was recorded between 126-148° C. (onset at 133.5° C., ΔH=−39.7 J/g) (FIG. 31). No interaction reported.

In summary, different types of interactions were observed among the excipients and the (R)-isometheptene mucate. A physical interaction was observed between mannitol and (R)-isometheptene mucate (in a ratio of 1:1). A physical interaction was observed with magnesium stearate (in a ratio of 1:1), probably due to the partial (R)-isometheptene mucate solubilization after excipient melting. Because in the final formulation these two excipients are present in very low amounts, the interaction could be considered unimportant or inconclusive. Lowering the degree of crystallinity of (R)-isometheptene mucate was observed with colloidal silicon. Table 3 summarizes the observations of the various excipients with (R)-isometheptene mucate API.

TABLE 3
Excipient reactions with (R)-isometheptene mucate

| Excipient | Mixture 1:1 |
|---|---|
| Mannitol SD₂₀₀ | Physical interaction (complex) |
| Magnesium Stearate | YES |
| | (physical interaction after melting) |
| Plasdone K29/32 | NO |
| Silicon colloidal | No interaction |
| | (reduction of crystallinity degree) |
| Crospovidone | NO |
| Isomalt | NO |

### Example 4

The compatibility of mannitol with (R)-isometheptene mucate was investigated by differential scanning calorimetry (DSC), and the resulting interactions were assessed. In particular, the formation of a eutectic between the mannitol and the (R)-isometheptene mucate during mixing improved the cohesion between the particles and provided better physical bonding between the (R)-isometheptene mucate active pharmaceutical ingredient (API) and the mannitol excipient.

The interaction between (R)-isometheptene mucate and mannitol SD₂₀₀ is an invariant physical interaction because it is in thermal equilibrium in which the two components are well mixed and stabilized. Physically, this means that the melted eutectic, solid eutectic, and solid mannitol all coexist at the same time and are in chemical equilibrium. The resulting solid macrostructure from the eutectic reaction depends on a few factors, including that the two solid solutions nucleate and grow together during a mechanical mixture.

Because mannitol is a common excipient in solid drug formulations, it was examined for compatibility with (R)-isometheptene mucate and investigated using DSC and the interactions occurring were assessed. Surprisingly, the formation of a eutectic during mechanical mixing was discovered. To confirm the formation of a eutectic and to characterize its physical properties, several binary mixtures at different ratios of (R)-isometheptene mucate and excipient were prepared and analyzed by DSC and by XRPD. The eutectic formation improved the cohesion between the (R)-isometheptene mucate and excipient particles and assured better physical linking between the two.

In order to confirm the eutectic formation and to characterize its physical properties, several binary mixtures at different ratios of (R)-isometheptene mucate-excipient were prepared and analyzed by DSC and by X-ray powder diffraction (XRPD). The mixtures were obtained by gently milling in agate mortar of micronized (R)-isometheptene mucate and mannitol, in order to obtain homogeneous distribution of the particles. For each DSC heating curve, the onset temperature and the enthalpy were evaluated both for the eutectic contribute and for the excess of component. The recorded values were plotted and a phase diagram between the two components was obtained with a characteristic profile of phase diagrams of eutectic mixtures.

Mixtures also were investigated by XRPD and compared with the patterns of pure components. These analyses were carried out to confirm that the eutectic compound is only a physical interaction between the two products and not a formation of a new entity with different chemical properties.

Aliquots of (R)-isometheptene mucate API and mannitol were weighed in the ratios described below and ground in an agate mortar, and the homogeneous mixtures subsequently analyzed.

| Mixture | API amount (%) |
|---|---|
| 1 | 0 |
| 2 | 25 |
| 3 | 40 |
| 4 | 50 |
| 5 | 60 |
| 6 | 75 |
| 7 | 90 |
| 8 | 100 |

### Differential Scanning Calorimetry (DSC)

DSC heating curves were obtained using a TA 821 DSC Mettler instrument under the following conditions:

| | |
|---|---|
| Heating rate | 10° C./min |
| Ambient | Nitrogen 30 mL/min |
| Sample order | Normal open aluminum pan |
| Temperature range | From 25° C. to 250° C. |
| Instrument calibration | Indium sample purity 99.999% |

### X-Ray Powder Diffraction (XRPD)

X-ray powder diffraction (XRPD) tests were performed with a ULTIMA IV (Rigaku) instrument, laying the sample on a static sample holder. The X-ray focusing slit had a variable width, interlocked with the 0 value. The X-ray tube had a copper target, with a current intensity of 40 mA and a voltage of 50 kV. The radiation generated by the Cockcroft-Walton method is constituted by K_{α1} (1.540562 Å) and K_{α2} (1.544398 Å). The analytical conditions were as follows:

Fixed Time: Sampling width, 0.02 deg; Scanning rate, 1.0 s/step

2θ range: 3/50 deg.
Sample holder: amorphous glass—equiangular 9200/2G, 0.2 mm deep. The sample was pressed with a glass plate.

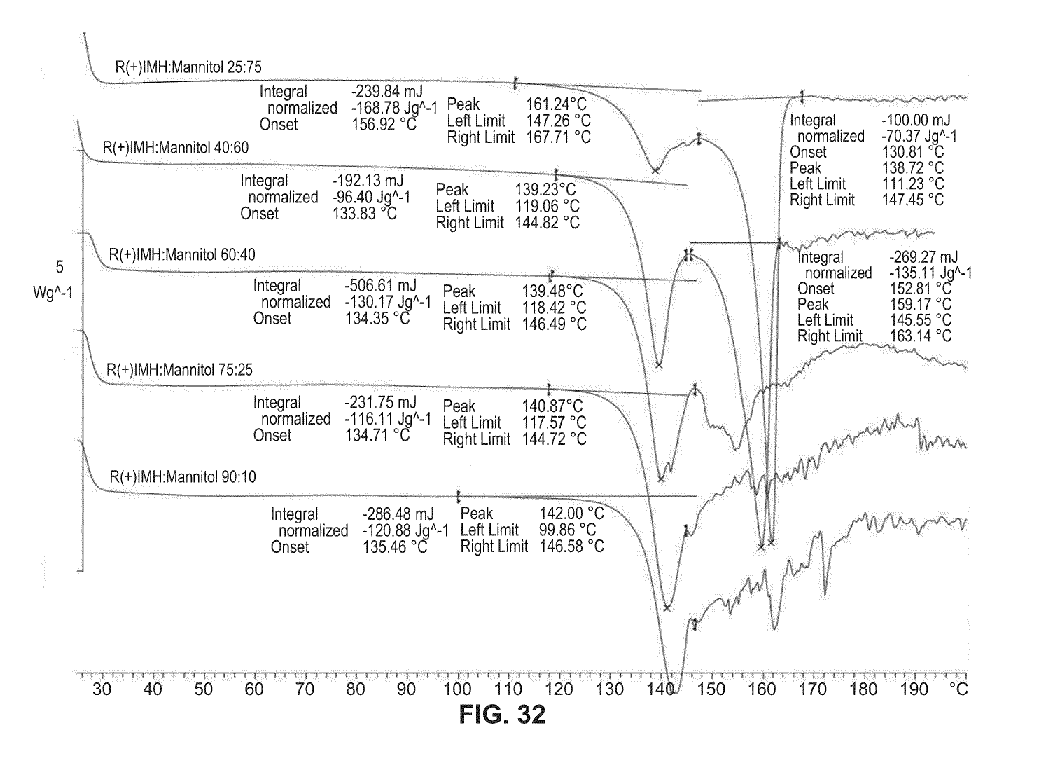

Pure components of (R)-isometheptene mucate and mannitol, as well as mixtures of the two, were analyzed with DSC. FIG. 18 depicts the melting curve with 100% (R)-isometheptene mucate. Melting with decomposition was detected starting from 129° C. (onset at 144° C.). FIG. 2 depicts the melting curve with 100% mannitol. Melting was detected between 151° C. and 172° C. (onset at 163.9° C., ΔH=−241.1 J/g). FIG. 32 depicts the various mixtures. Table 4 summarizes the data.

TABLE 4
Summary of DSC data

| | | | T onset | | ΔH | | |
|---|---|---|---|---|---|---|---|
| | % | T onset | 2nd | ΔH | 2nd | ΔH | |
| % | Man- | eutectic | transition | eutectic | effect | global | |
| API | nitol | (° C.) | (° C.) | (J/g) | (J/g) | (J/g) | Plot |
| 0 | 100 | — | 163.86 | — | — | 241.07 | FIG. 2 |
| 25 | 75 | 130.81 | 156.92 | 70.37 | 168.78 | — | FIG. 32 |
| 40 | 60 | 133.83 | 152.81 | 96.40 | 135.11 | — | FIG. 32 |
| 50 | 50 | 134.88 | 150.27 | 93.39 | 113.19 | — | FIG. 26 |
| 60 | 40 | 134.35 | — | 130.17 | — | — | FIG. 32 |
| 75 | 25 | 134.71 | — | 116.11 | — | — | FIG. 32 |
| 90 | 10 | 135.46 | — | 120.88 | — | — | FIG. 32 |
| 100 | 0 | — | 144.26 | — | — | 267.93 | FIG. 18 |

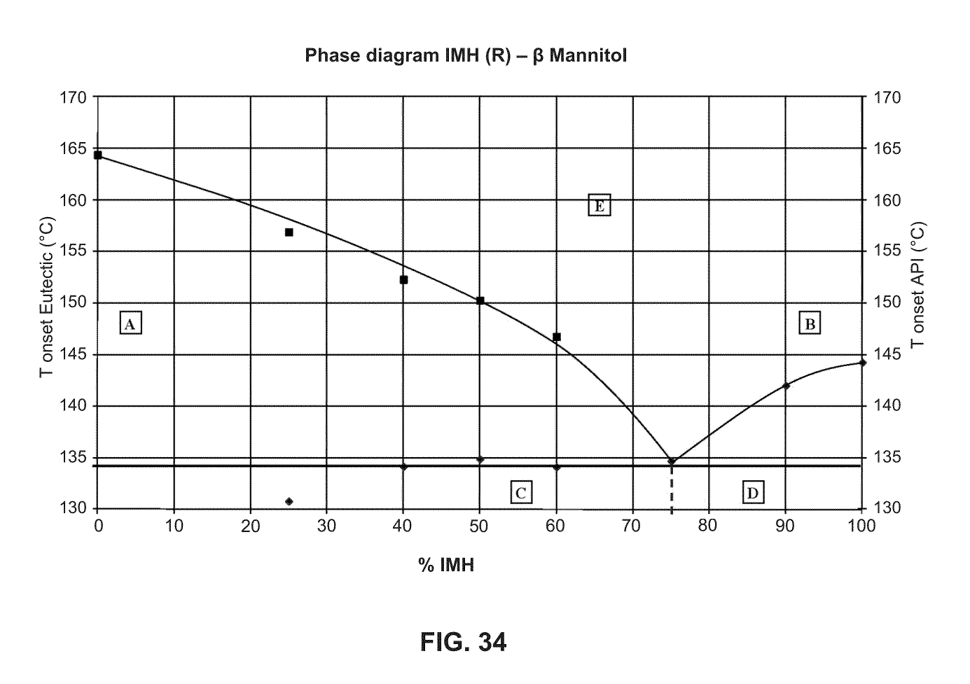

The above results demonstrated that the eutectic composition formed at approximately 75% (R)-isometheptene mucate (API) and 25% mannitol. Under approximately 75%, two distinct melting peaks were observed from the melting of the eutectic fraction and the excess of the individual components. FIG. 34 shows a phase diagram depicting the onset melting temperatures of the eutectic fraction and the excess components, plotted as function of (R)-isometheptene mucate percentage. Five distinct zones are present in the diagram:

Zone A: Excess of mannitol (liquid mannitol+solid eutectic)
Zone B: Excess of (R)-isometheptene mucate (solid eutectic+liquid (R)-isometheptene mucate)
Zone C: Solid eutectic with solid mannitol
Zone D: Solid eutectic with solid (R)-isometheptene mucate
Zone E: Liquid phase with mannitol and (R)-isometheptene mucate

In Zone A, when the percentage of (R)-isometheptene mucate increased, the onset temperature of the excess of mannitol decreased while the temperature of eutectic fraction remained constant around 134.7° C. Above the eutectic composition, the excess of (R)-isometheptene mucate led to an increase in the temperature (Zone B). In addition, there was a good correlation between mixtures and temperature. A few small deviations from the trend curve were due to an incompletely homogeneous powder mixture.

### XRPD

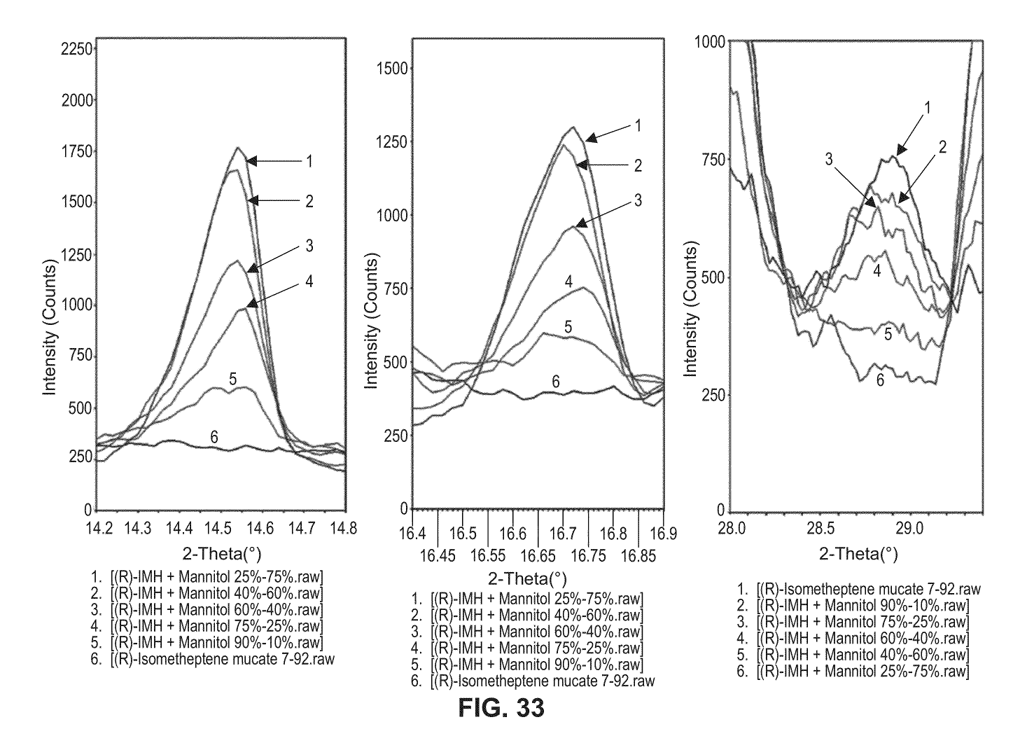

To confirm that the eutectic composition was only a physical mixture and that a new entity or adduct was not formed, the mixtures were analyzed by X-ray Powder Diffraction, where no thermal treatments were applied [pure (R)-isometheptene mucate, FIG. 33; pure mannitol, FIG. 4]. FIG. 33 shows the stacking of pure (R)-isometheptene mucate and mixtures of mannitol and (R)-isometheptene mucate, where it was possible to point out three distinct diffraction peaks: 14.5° 2θ, 16.7° 20 and 28.8° 20.

In summary, the data show that thermal behavior of the mixtures presents two endotherms, relating to the eutectic and to the melting of the excess of the main component. Thermal entities recorded for the mixtures agreed with the percentage of (R)-isometheptene mucate/mannitol ratio present in the eutectic mixture. At the eutectic composition, only one melting peak was visible. The eutectic composition was reached at about 75% (R)-isometheptene mucate and 25% mannitol. The eutectic composition confirmed the molar stoichiometry (ratio between the two components: 1.0:1.0). The melting temperature of the eutectic was about 134° C. and was recorded for all the investigated mixtures. By XRPD, no adduct interaction occurred between (R)-isometheptene mucate and mannitol, only a physical eutectic formation.

### Example 5

A mixture of racemic isometheptene mucate and (R)-isometheptene mucate were investigated by differential scanning calorimetry (DSC) to identify the different behaviors of the racemic isometheptene mucate and (R)-isometheptene mucate.

Mixtures also were investigated by XRPD and compared with the patterns of pure components.

Aliquots of (R)-isometheptene mucate (API) and racemic isometheptene mucate were weighed in the ratios described below and ground in an agate mortar, and the homogeneous mixtures subsequently analyzed.

| Mixture | (R)-IMH (%) |
|---|---|
| 1 | 0 |
| 2 | 10 |
| 3 | 25 |
| 4 | 37.5 |
| 5 | 45 |
| 6 | 50 |
| 7 | 70 |
| 8 | 75 |
| 9 | 100 |

### Differential Scanning Calorimetry (DSC)

DSC heating curves were obtained using a TA 821 DSC Mettler instrument under the following conditions:

| | |
|---|---|
| Heating rate | 10° C./min |
| Ambient | Nitrogen 30 mL/min |
| Sample order | Normal open aluminum pan |
| Temperature range | From 25° C. to 250° C. |
| Instrument calibration | Indium sample purity 99.999% |

### X-Ray Powder Diffraction (XRPD)

X-ray powder diffraction (XRPD) tests were performed with a ULTIMA IV (Rigaku) instrument, laying the sample on a static sample holder. The X-ray focusing slit had a variable width, interlocked with the 0 value. The X-ray tube had a copper target, with a current intensity of 40 mA and a voltage of 50 kV. The radiation generated by the Cockcroft-Walton method is constituted by K_{α1} (1.540562 Å) and K_{α2} (1.544398 Å). The analytical conditions were as follows:

Fixed Time: Sampling width, 0.02 deg; Scanning rate, 1.0 s/step
2θ range: 3/50 deg.
Sample holder: amorphous glass—equiangular 9200/2G, 0.2 mm deep. The sample was pressed with a glass plate.

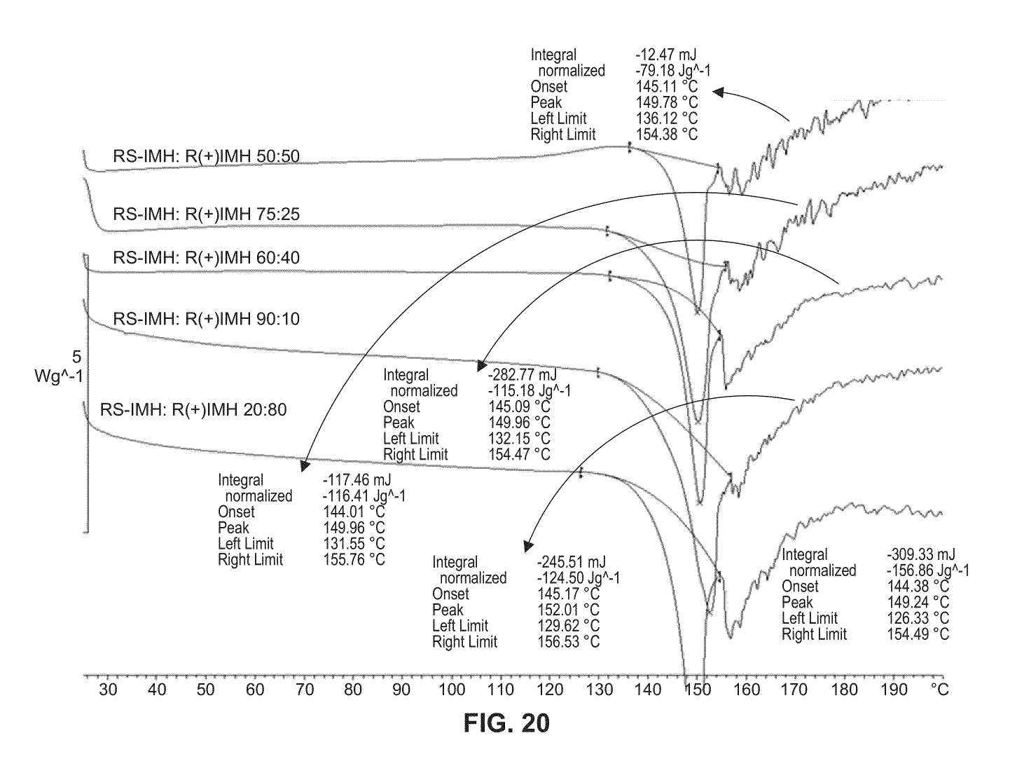

Pure components of (R)-isometheptene mucate and racemic isometheptene mucate, as well as mixtures of the two, were analyzed with DSC. FIG. 18 depicts the melting curve with 100% (R)-isometheptene mucate. Melting with decomposition was detected starting from 129° C. (onset at 144° C.). FIG. 18 depicts the melting curve with 100% racemic isometheptene mucate. Melting with decomposition was detected starting from 130° C. (onset at 147° C.). FIG. 20 depicts the various mixtures. Table 5 summarizes the data.

TABLE 5
Summary of DSC data

| | | | ΔH | |
|---|---|---|---|---|
| % (R)- | % (RS)- | T onset | global | |
| IMH | IMH | (° C.) | (J/g) | Plot |
| 0 | 100 | 146.87 | 286.43 | FIG. 18 |
| 10 | 90 | 145.17 | 124.50 | FIG. 20 |
| 25 | 75 | 144.01 | 116.41 | FIG. 20 |
| 40 | 60 | 145.09 | 115.18 | FIG. 20 |
| 50 | 50 | 145.11 | 79.18 | FIG. 20 |
| 80 | 20 | 144.38 | 156.86 | FIG. 20 |
| 100 | 0 | 144.26 | 267.93 | FIG. 18 |

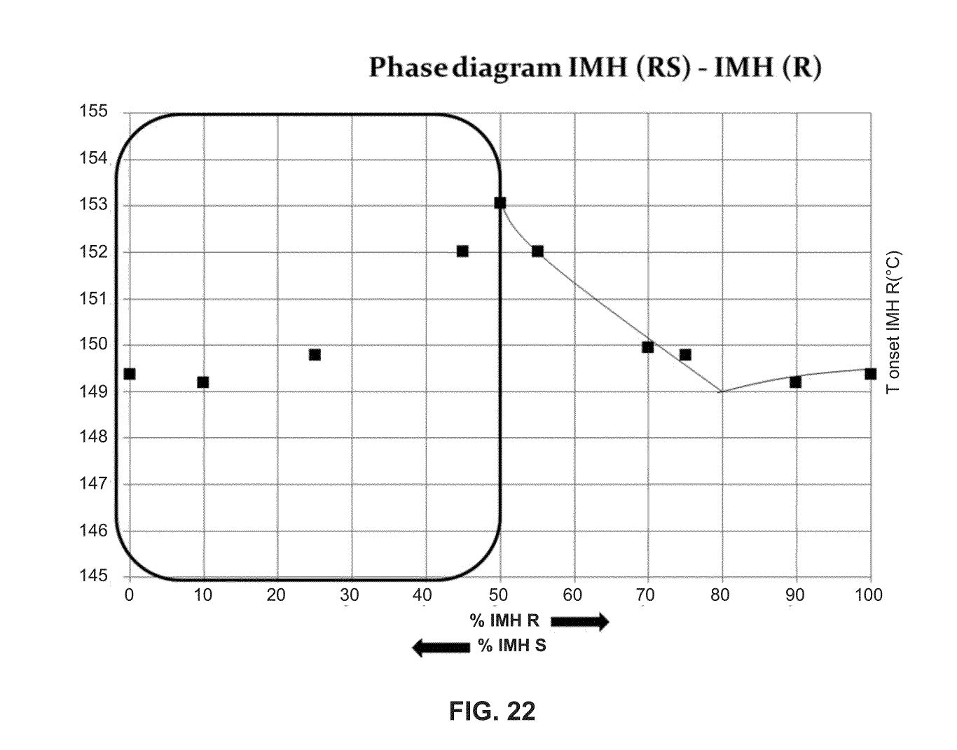

The above results demonstrated that the lowest melting point was found at approximately 80% (R)-isometheptene mucate (API) and 20% (S)-isometheptene mucate and the highest melting point found at 50% (R)-isometheptene and 50% (S)-isometheptene, this racemic mixture corresponds to a so called “racemate”. The data collected confirms that the racemic mixture is a racemate and not a “conglomerate” as expected for the same crystal structure of each pure enantiomer. FIG. 22 shows a phase diagram depicting the onset melting temperatures, plotted as function of (R)-isometheptene mucate percentage.

### XRPD

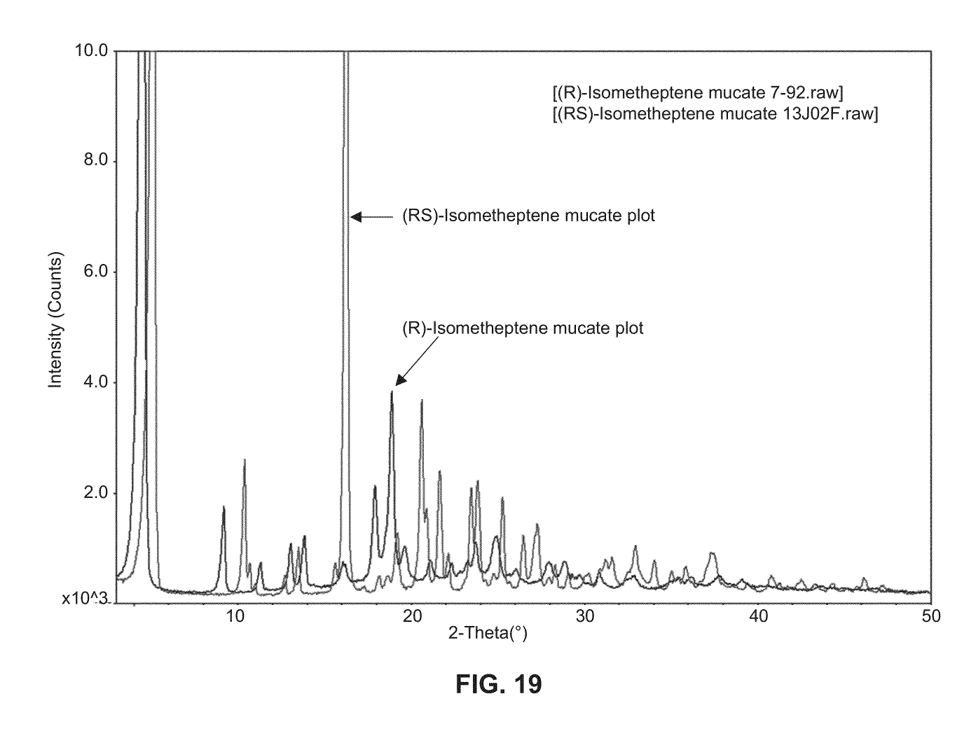

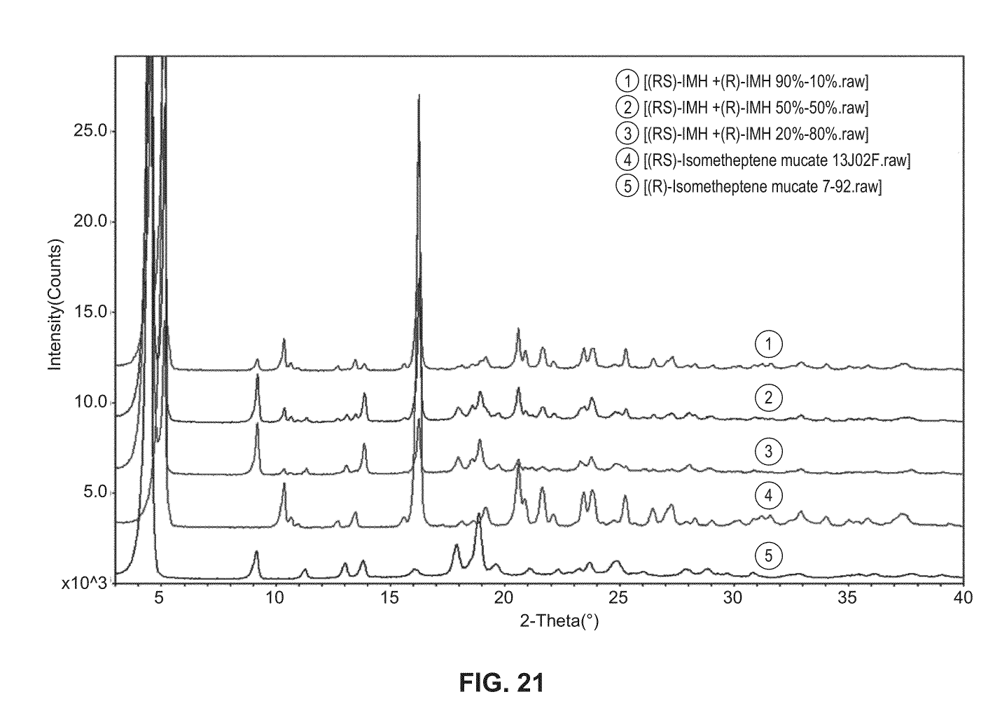

The mixtures were analyzed by X-ray Powder Diffraction, where no thermal treatments were applied (pure (R)-isometheptene mucate, FIG. 19; pure racemic isometheptene mucate, FIG. 19). FIG. 21 shows the stacking of pure (R)-isometheptene mucate, pure racemic isometheptene mucate and mixtures thereof. No other interactions were observed, but it was confirmed that as expected for a “racemate,” the racemic mixture corresponds to a different crystal structure than that of the pure enantiomer (R)-isometheptene mucate (API).

### Example 6

Thermal analytical techniques were used to analyze various methods of preparation of a drug product containing (R)-isometheptene mucate (API) and mannitol in a ratio of 75:25. Based on the thermal events recorded for each component and for the mixtures, the analyses were carried out by investigating the peaks recorded by differential scanning calorimetry (DSC) in mixture between (R)-isometheptene mucate and the excipients. Differences in thermal profiles between the single compound and the related mixture obtained after milling the products in an agate mortar (dry-granulation), milling the product in an agate mortar with a small amount of water (wet-granulation), preparing the sample with fast evaporation with 1:1 water:ethanol, and preparing the sample with spray dry (SD, mixture in 1:1 water:ethanol) were evaluated.

The following raw materials were used:

(R)-isometheptene mucate

### Mannitol SD₂₀₀

### Water

### Ethanol

Aliquots of (R)-isometheptene mucate and mannitol were weighed in a ratio of 75:25 and ground in an agate mortar (dry-granulation), ground in an agate mortar with a small amount of water (wet-granulation), prepared using fast evaporation with 1:1 water:ethanol, or prepared using spray dry (mixture in 1:1 water:ethanol). The homogeneous mixtures then were analyzed.

### Differential Scanning Calorimetry (DSC)

The DSC heating curves were obtained with a TA 821 DSC Mettler instrument under the following conditions:

Heating rate: 10° C./min
Ambient: Nitrogen 30 mL/min
Sample holder: normal open aluminum pan
Temperature range: from 25° C. to 250° C.
Instrument calibration: Indium sample purity 99.999%

### X-Ray Powder Diffraction (XRPD)

X-ray powder diffraction (XRPD) tests were performed with the ULTIMA IV instrument (Rigaku), laying the sample on a static sample holder. The X-ray focusing slit has a variable width, interlocked with the q value. The X-ray tube has a copper target, with a current intensity of 40 mA and a voltage of 40 kV. Radiation was generated by the Cockcroft-Walton method, and was constituted by K_{α1} (1.540562 Å) and K_{α2} (1.544398 Å). The analytical conditions were:

Fixed Time; sampling width 0.02 deg, scanning rate 1.3 s/step, 2 q range 3.35 deg and sample holder; amorphous glass equiangular 9200/2G, 0.2 mm deep. The sample was pressed with a glass plate.

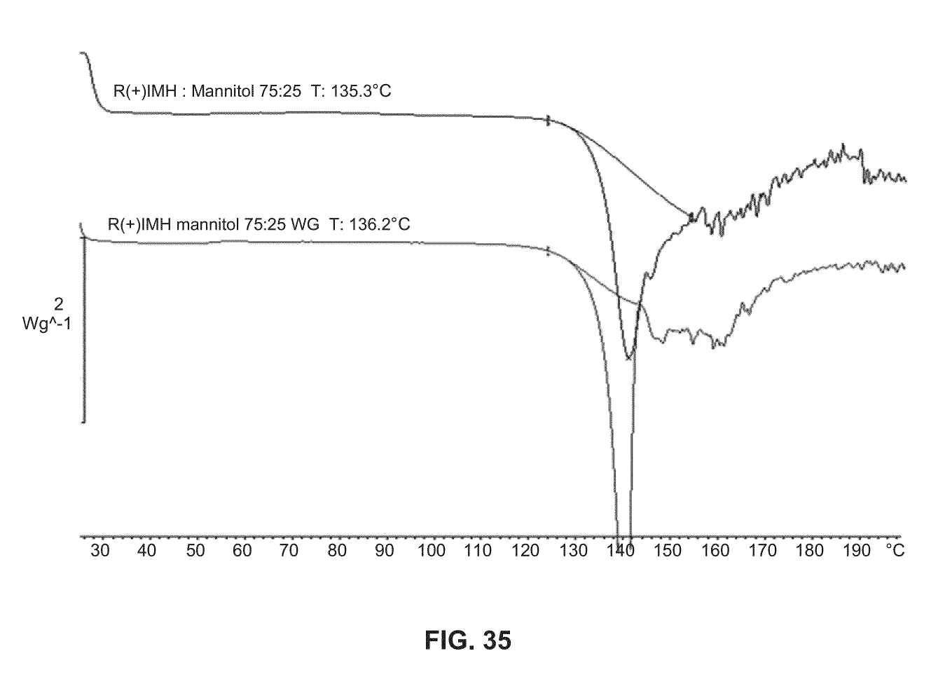

In a 75:25 eutectic mixture of (R)-isometheptene mucate and mannitol prepared by dry-granulation in an agate mortar the onset of melting was observed at 135.3° C. (FIG. 35). If a small amount of water was added to the mixture during granulation (wet-granulation), the onset of melting for the resulting mixture was observed at 136.2° C. (FIG. 35).

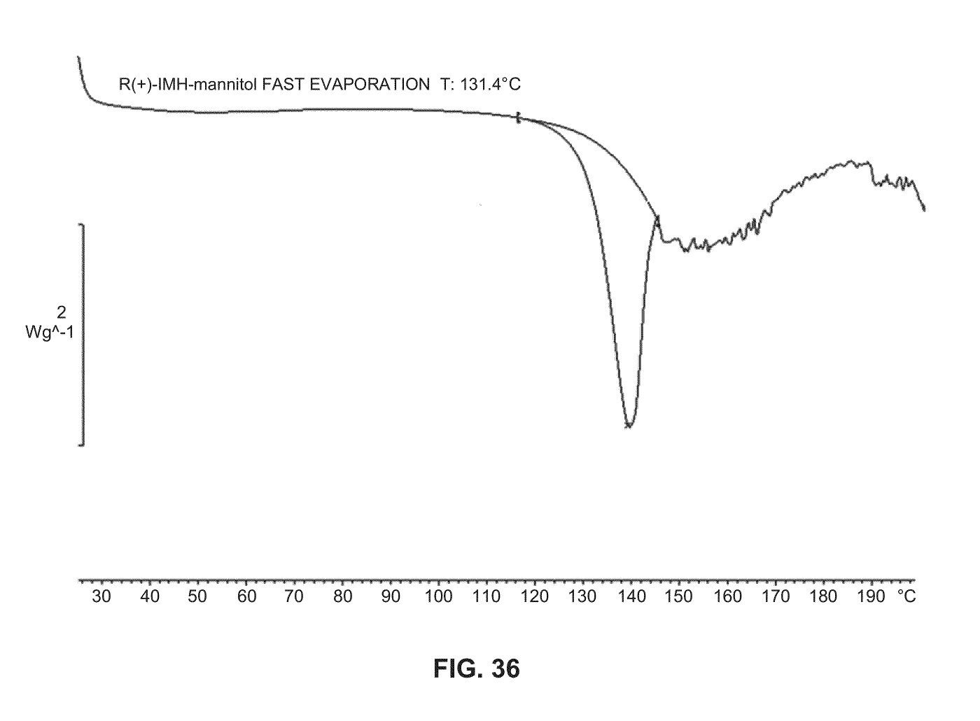

In a 75:25 eutectic mixture of (R)-isometheptene mucate and mannitol prepared by fast evaporation of a solution of the mixture in 1:1 water:ethanol, the onset of melting was observed at 131.4° C. (FIG. 36).

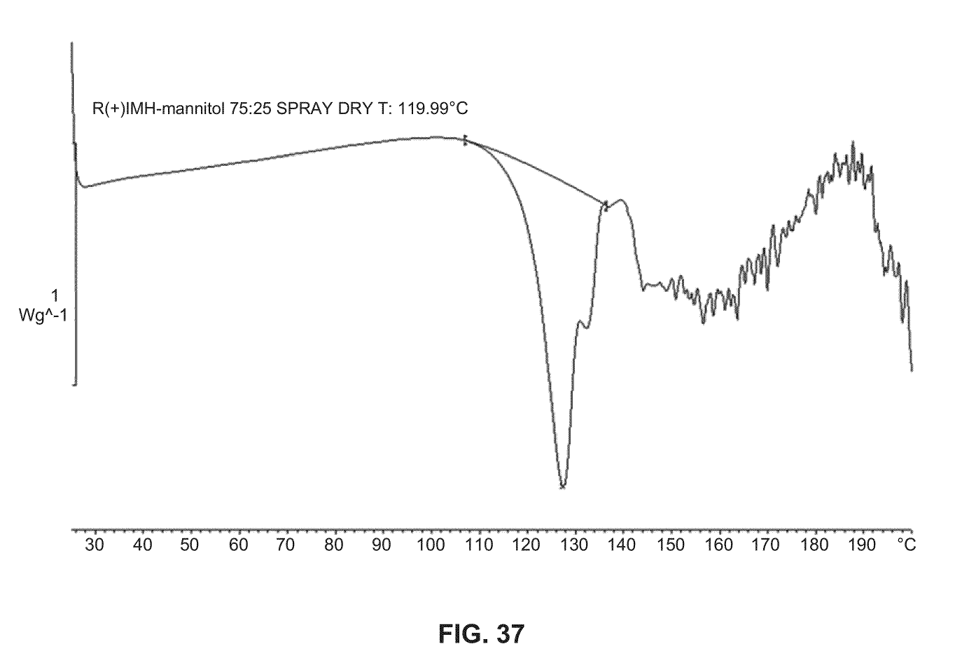

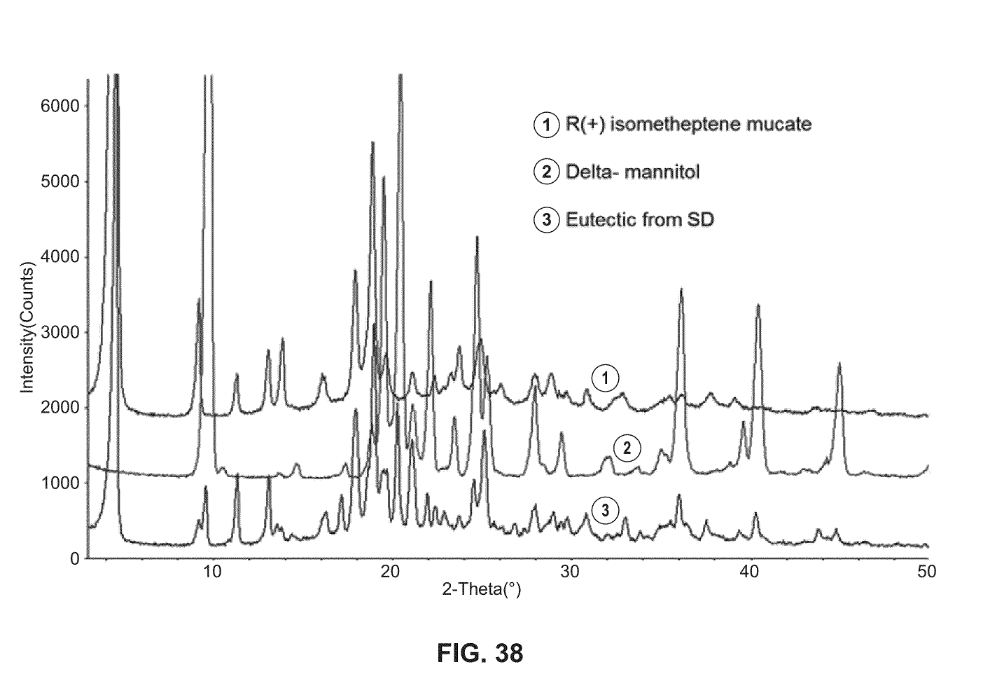

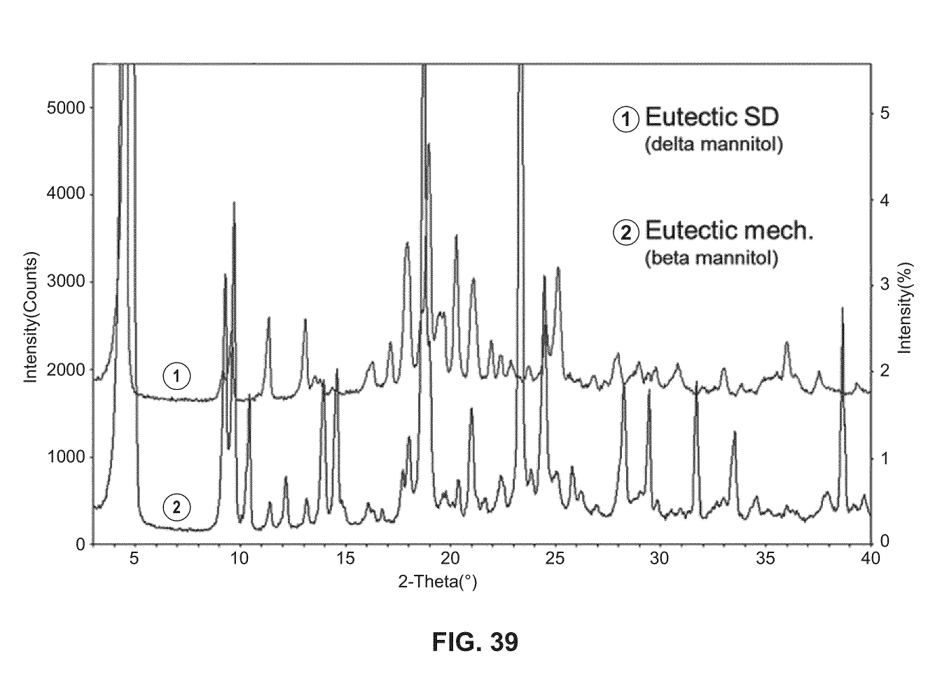

In a 75:25 eutectic mixture of (R)-isometheptene mucate and mannitol prepared by spray drying of a solution of the mixture in 1:1 water:ethanol, the onset of melting was observed at 119.99° C. (FIG. 37). This formulation generates a eutectic with δ-mannitol instead of β-mannitol. XRPD analysis of the sample prepared by spray drying confirmed the physical interaction between δ-mannitol and (R)-isometheptene mucate (FIG. 38). In FIG. 39, a comparison of the XRPD patterns of a eutectic obtained by spray dry and a eutectic obtained by dry-granulation is reported. The eutectic obtained after the spray dry process shows a eutectic with δ-mannitol.

The above results confirmed that the eutectic composition formed at approximately 75% (R)-isometheptene mucate and 25% mannitol regardless of the method of eutectic preparation. Preparation of the formulation using the spray dry technique led to formulation of a eutectic between δ-mannitol and (R)-isometheptene mucate. The δ-mannitol and (R)-isometheptene mucate eutectic had a lower melting temperature than the β-mannitol and (R)-isometheptene mucate eutectic, an advantage over the β-mannitol eutectic.

### Example 7

Thermal analytical techniques were used to analyze mixtures of several (R)-isometheptene salts and β-mannitol in a ratio of 75:25. (R)-isometheptene salts tested were as follows: (R)-isometheptene maleate, (R)-isometheptene malate, and (R)-isometheptene tartrate. Based on the thermal events recorded for each component and for the mixtures, the analyses were carried out by investigating the peaks recorded by differential scanning calorimetry (DSC) in mixture between (R)-isometheptene mucate and the excipients. Differences in thermal profiles between the single compound and the related mixture were evaluated.

The following raw materials were used:

(R)-isometheptene malate
(R)-isometheptene maleate
(R)-isometheptene tartrate

### β-Mannitol SD₂₀₀

### Water

Aliquots of (R)-isometheptene salts and each excipient were weighed in a ratio of 1:1 (unless specified otherwise) and ground in an agate mortar with (wet-granulation) or without (dry-granulation) a small amount of water. The homogeneous mixtures then were analyzed.

### Differential Scanning Calorimetry (DSC)

The DSC heating curves were obtained with a TA 821 DSC Mettler instrument under the following conditions:

Heating rate: 10° C./min
Ambient: Nitrogen 30 mL/min
Sample holder: normal open aluminum pan
Temperature range: from 25° C. to 250° C.
Instrument calibration: Indium sample purity 99.999%

### X-Ray Powder Diffraction (XRPD)

X-ray powder diffraction (XRPD) tests were performed with the ULTIMA IV instrument (Rigaku), laying the sample on a static sample holder. The X-ray focusing slit has a variable width, interlocked with the q value. The X-ray tube has a copper target, with a current intensity of 40 mA and a voltage of 40 kV. Radiation was generated by the Cockcroft-Walton method, and was constituted by K_{α1} (1.540562 Å) and K_{α2} (1.544398 Å). The analytical conditions were:

Fixed Time; sampling width 0.02 deg, scanning rate 1.3 s/step, 2 q range 3.35 deg and sample holder; amorphous glass equiangular 9200/2G, 0.2 mm deep. The sample was pressed with a glass plate.

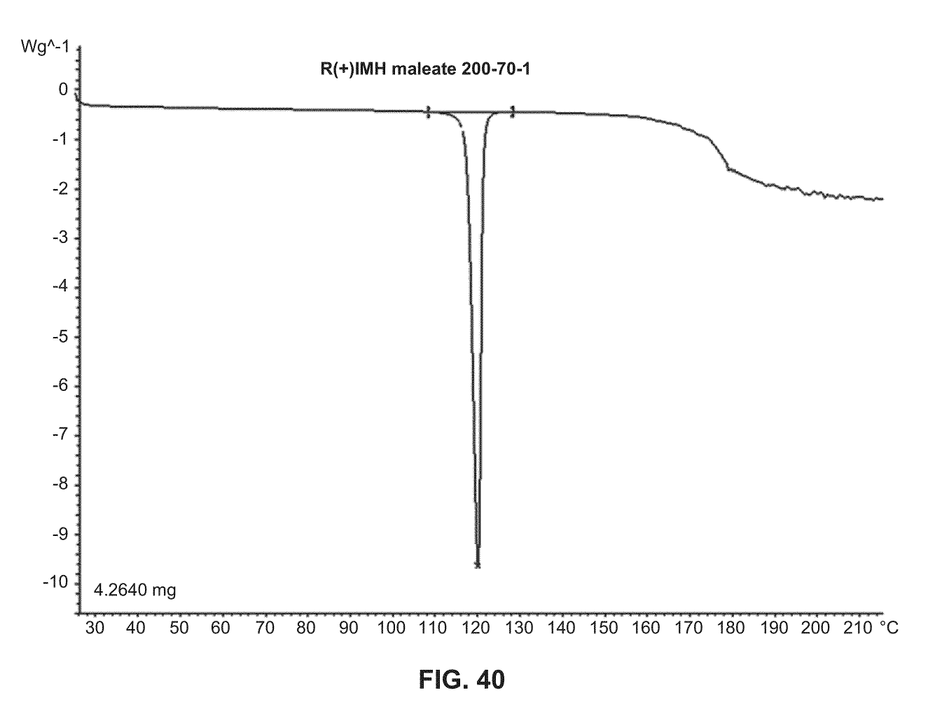

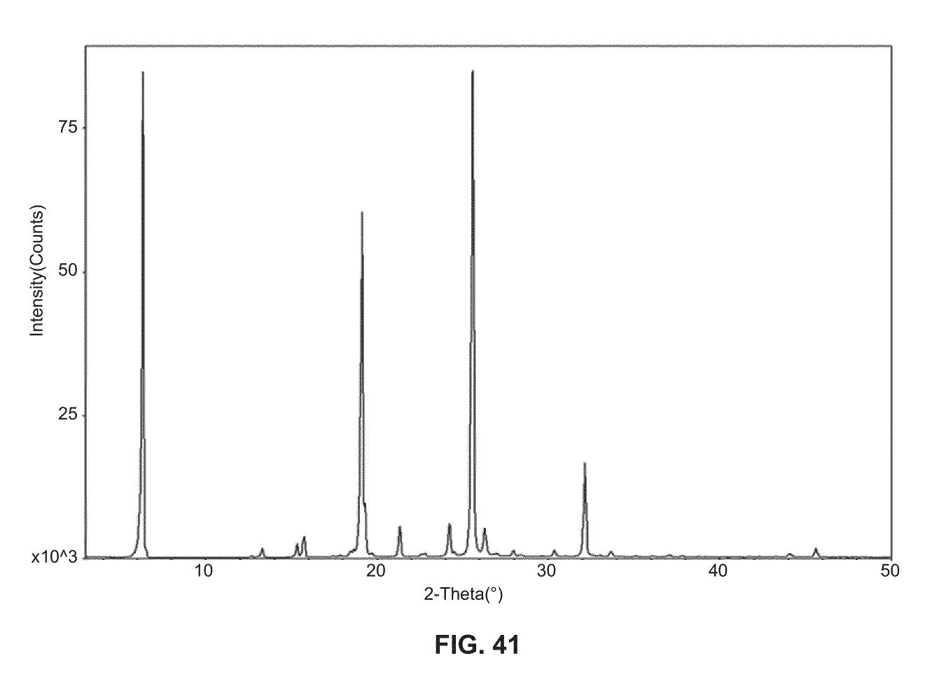

In a sample of (R)-isometheptene maleate, the onset of melting was observed at 117.1° C. (FIG. 40). In FIG. 41, the XRPD pattern is reported

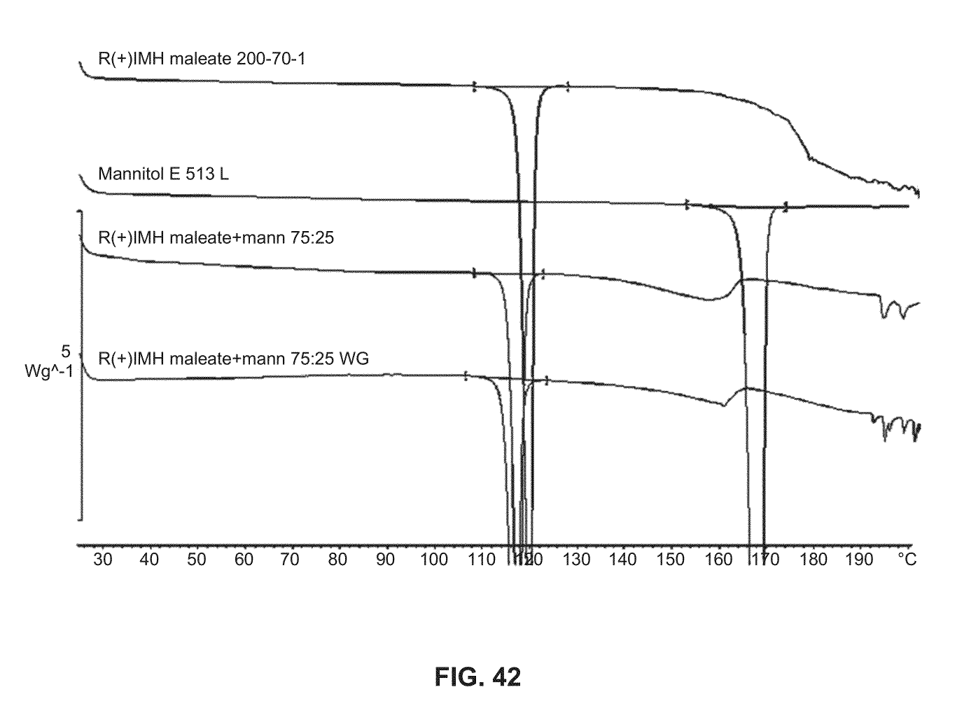

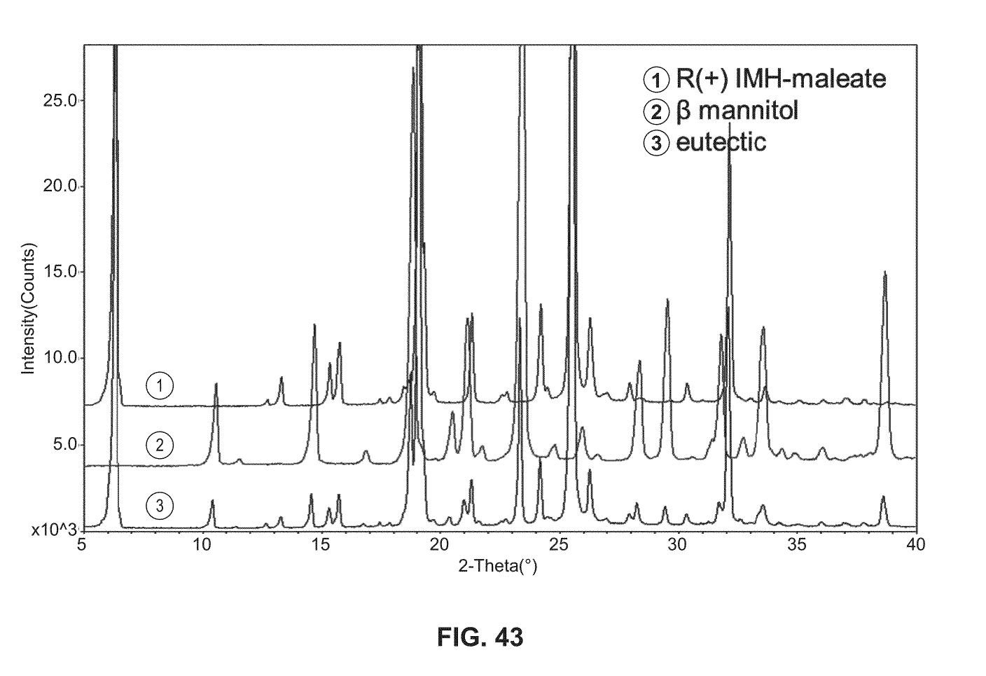

In a 75:25 mixture of (R)-isometheptene maleate and β-mannitol, dry-granulated or wet-granulated, the onset of melting was observed at 115.1° C. (FIG. 42). No physical interaction was observed as confirmed by XRPD (FIG. 43).

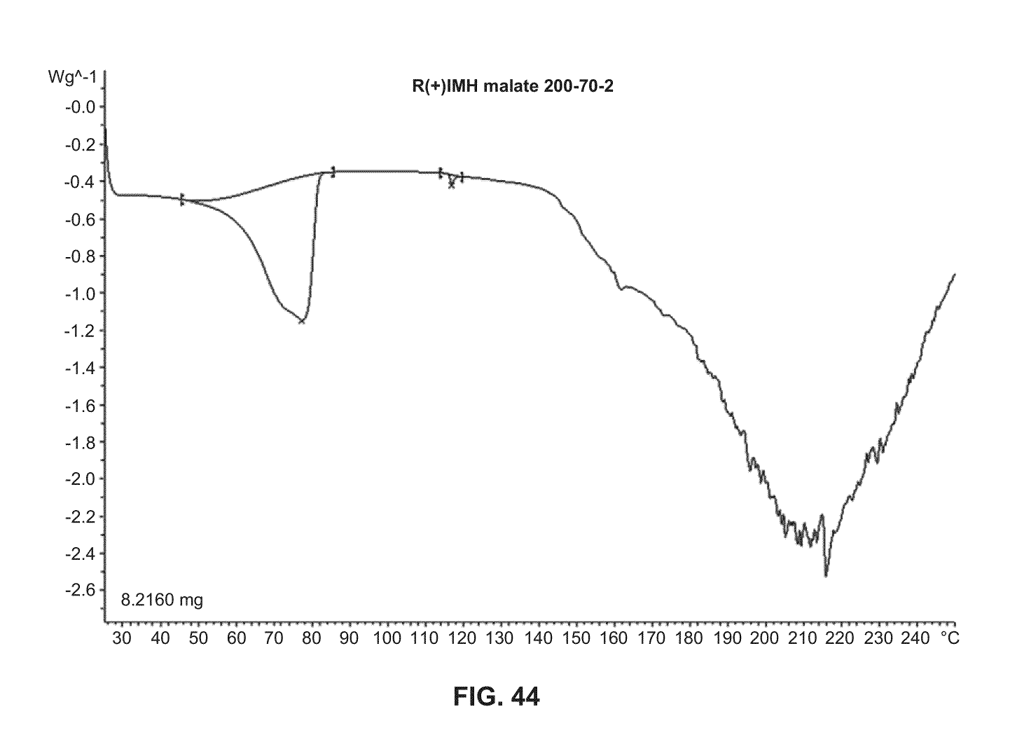

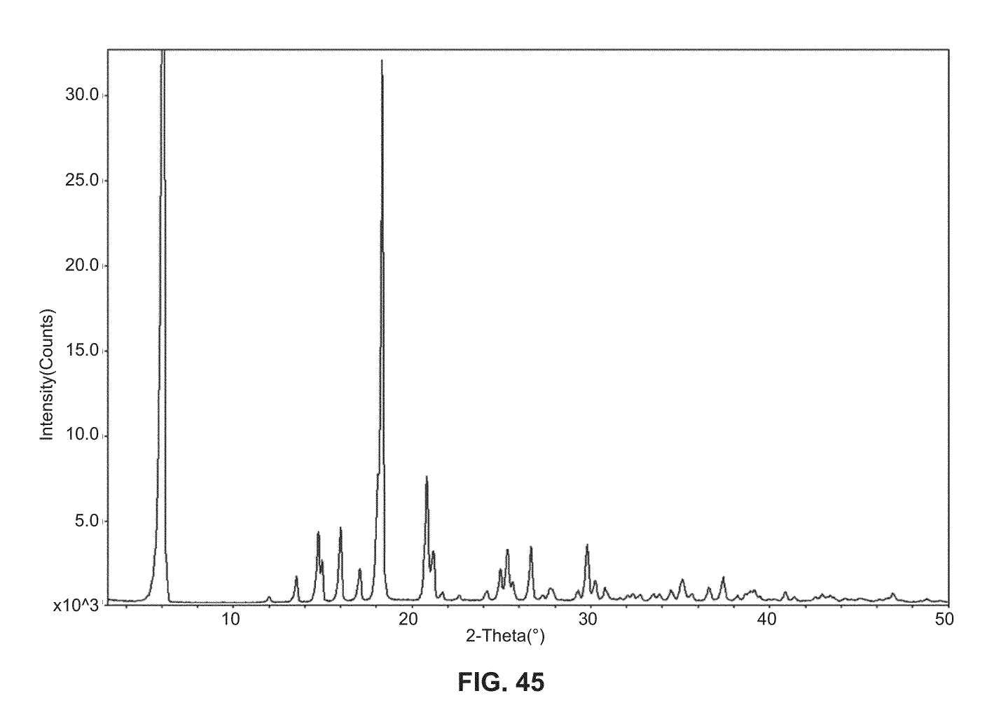

In a sample of (R)-isometheptene malate, the onset of melting was observed at 59.7° C. (FIG. 44). In FIG. 45, the XRPD pattern is reported.

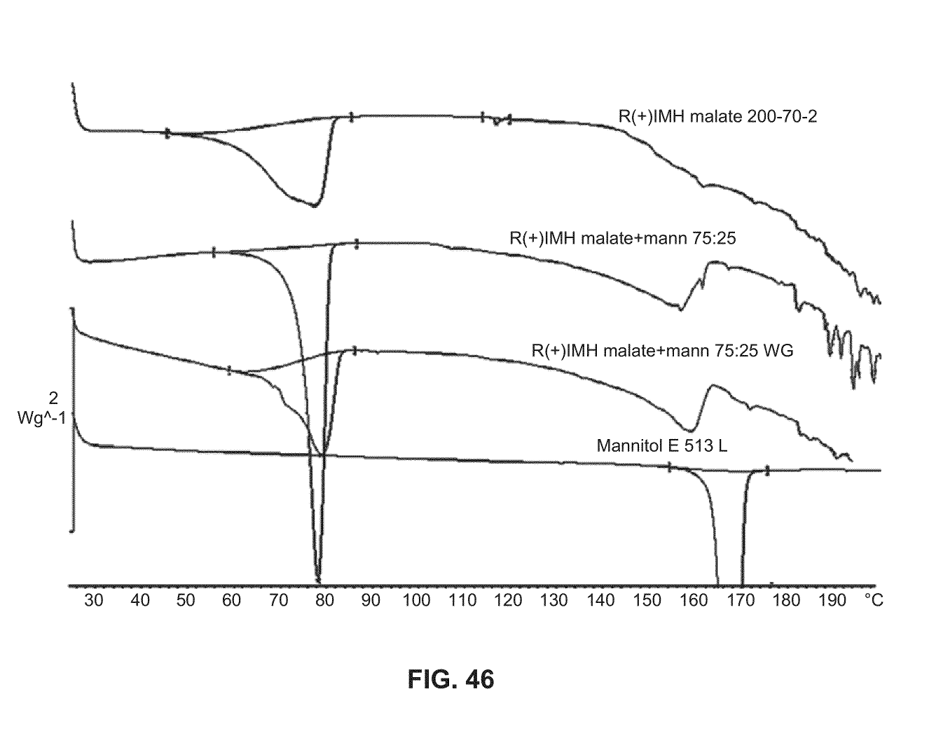

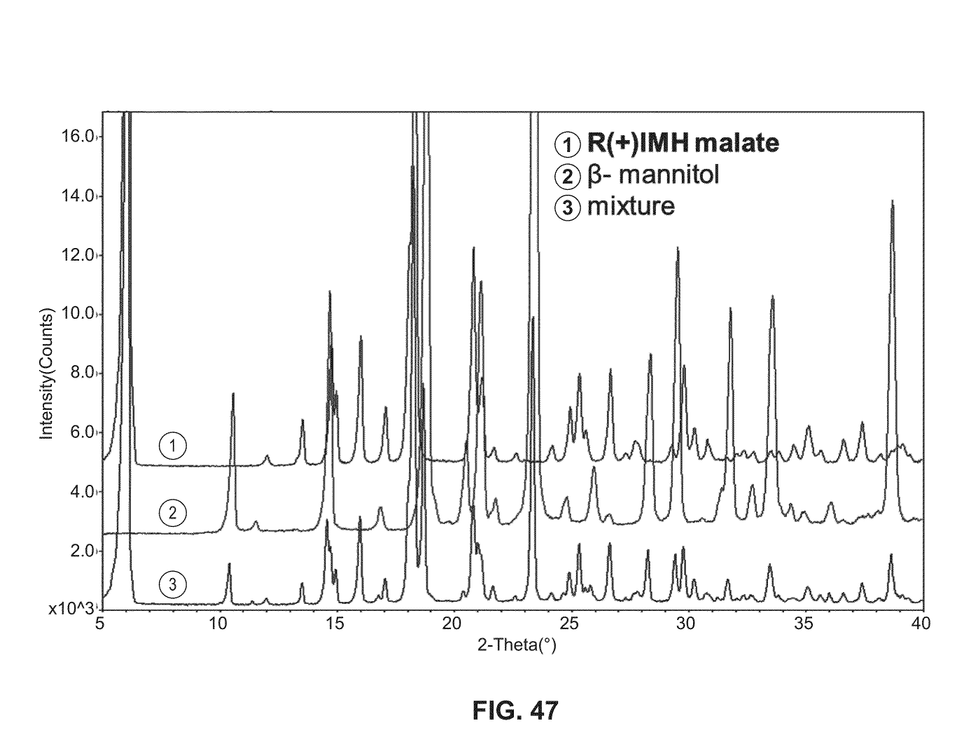

In a 75:25 mixture of (R)-isometheptene malate and β-mannitol, dry-granulated or wet-granulated, the onset of melting was observed at 74° C. (FIG. 46). No physical interaction was observed as confirmed by XRPD (FIG. 47).

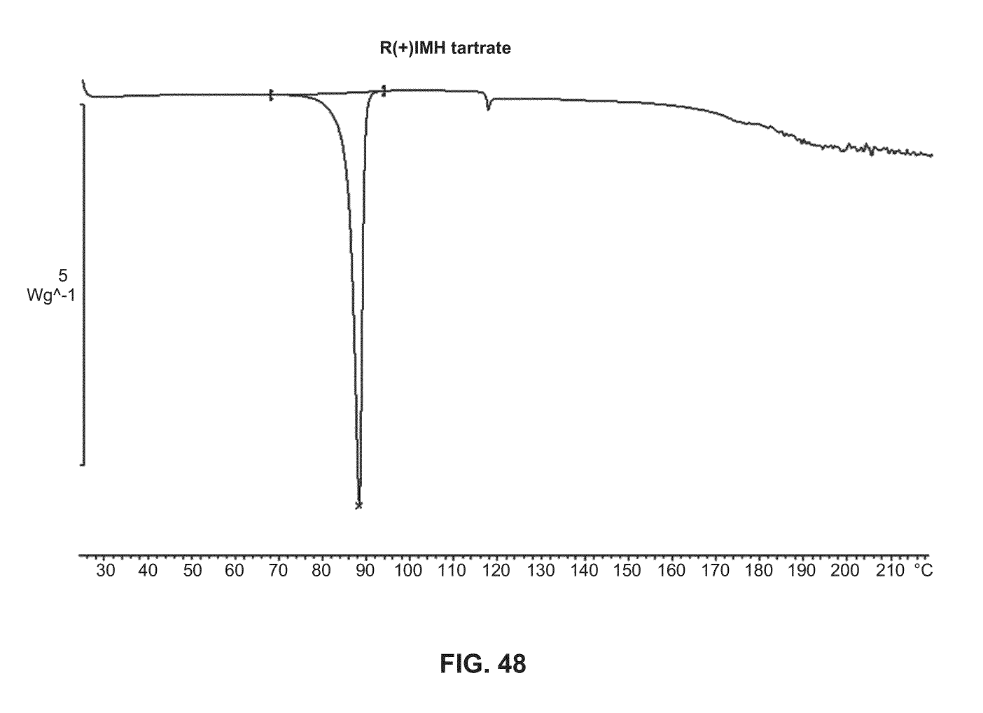

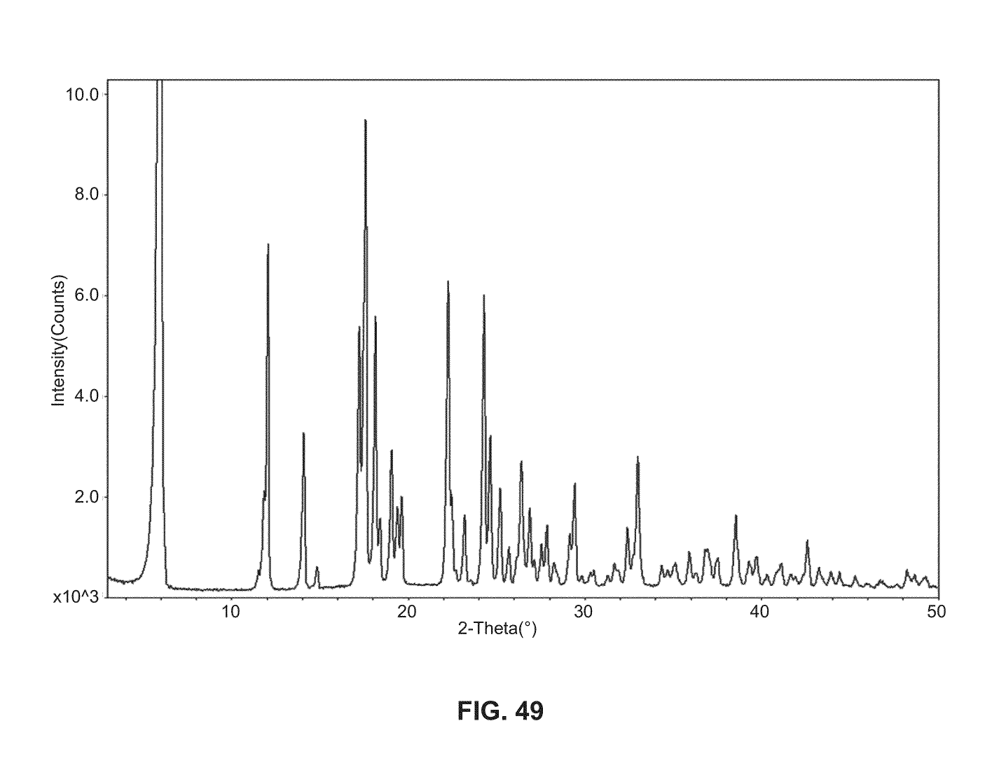

In a sample of (R)-isometheptene tartrate, the onset of melting was observed at 85.5° C. (FIG. 48). In FIG. 49, the XRPD pattern is reported.

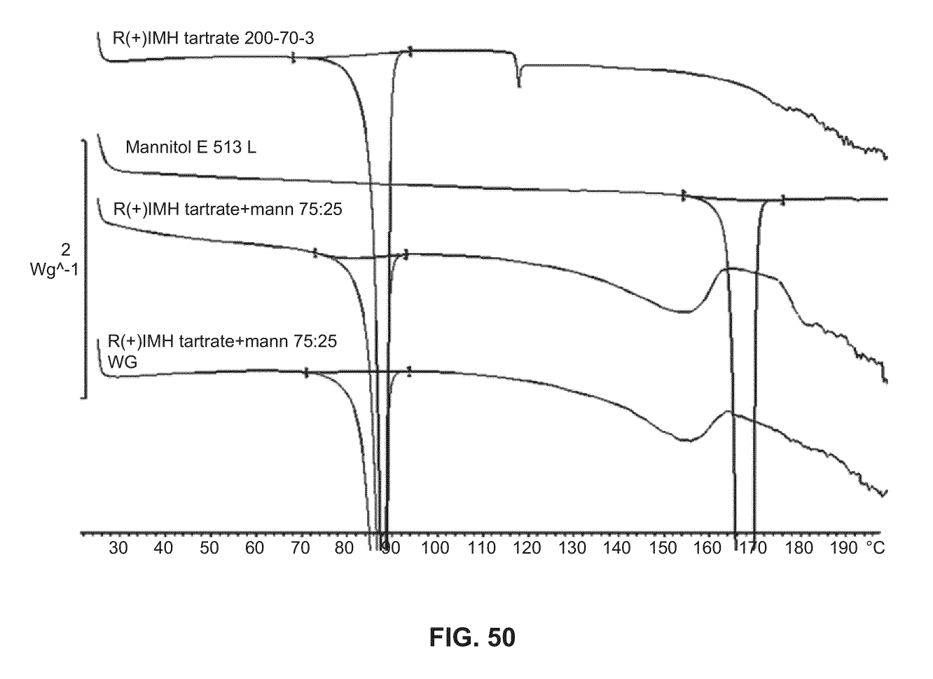

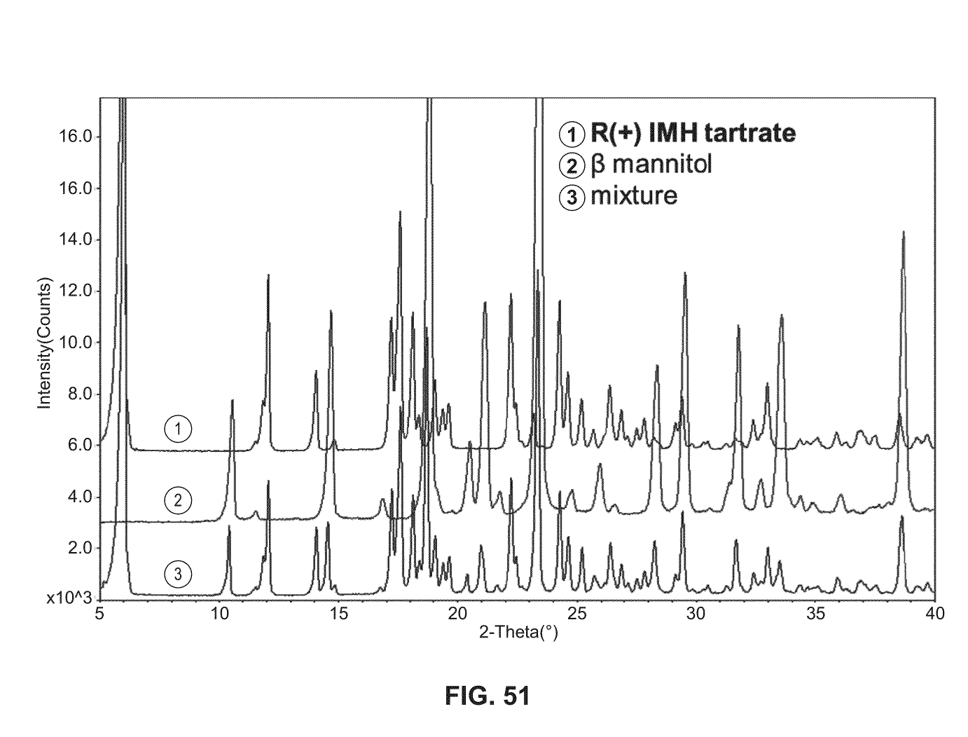

In a 75:25 eutectic mixture of (R)-isometheptene tartrate and mannitol, the onset of melting was observed at 84.7° C. (FIG. 50). No physical interaction was observed as confirmed by XRPD (FIG. 51).

The above results illustrated that (R)-isometheptene tartrate, (R)-isometheptene maleate, and (R)-isometheptene malate do not form a eutectic composition with mannitol. Without wishing to be bound by theory, it is possible that the extra hydroxyl groups on mucate compared to maleate, malate, or tartrate may interact more strongly with mannitol and are necessary in the formation of the eutectic.

### Example 8

Thermal analytical techniques were used to assess the stability of drug products (tablets) containing (R)-isometheptene mucate (API) and β-mannitol. The solid state characterization of different tablets obtained from wet-granulation with an agate mortar were assessed by thermal analysis (DSC) and XRPD techniques in order to confirm the eutectic formation and its physical properties. Stability and compatibility were assessed on the final drug product after stress conditions at 50° C. for one month.

The following formulations (tablets) prepared by wet-granulation were analyzed:

Formulation 1:
Formulation 1

| | |
|---|---|
| R-IMH Mucate | 61.0 mg |
| PVP | 2.1 mg |
| Mannitol PF | 40.7 mg |
| Mannitol SD₂₀₀ | 80.6 mg |
| Silica Colloidal | 4.6 mg |
| Crospovidone | 6.4 mg |
| Magnesium Stearate | 4.6 mg |
| Total | 200.0 mg |

Formulation 2:
Formulation 2

| | |
|---|---|
| R-IMH Mucate | 61.0 mg |
| PVP | 2.1 mg |
| Mannitol PF | 40.7 mg |
| Mannitol SD₂₀₀ | 10.0 mg |
| Methocel E3 | 10.0 mg |
| Methocel K100 | 90.0 mg |
| Silica Colloidal | 4.0 mg |
| Magnesium Stearate | 4.2 mg |
| Total | 222.0 mg |

### Differential Scanning Calorimetry (DSC)

The DSC heating curves were obtained with a TA 821 DSC Mettler instrument under the following conditions:

Heating rate: 10° C./min
Ambient: Nitrogen 30 mL/min
Sample holder: normal open aluminum pan
Temperature range: from 25° C. to 250° C.
Instrument calibration: Indium sample purity 99.999%

### X-Ray Powder Diffraction (XRPD)

X-ray powder diffraction (XRPD) tests were performed with the ULTIMA IV instrument (Rigaku), laying the sample on a static sample holder. The X-ray focusing slit has a variable width, interlocked with the q value. The X-ray tube has a Copper target, with a current intensity of 40 mA and a voltage of 40 kV. Radiation was generated by the Cockcroft-Walton method, and was constituted by K_{α1} (1.540562 Å) and K_{α2} (1.544398 Å). The analytical conditions were:

Fixed Time; sampling width 0.02 deg, scanning rate 1.3 s/step, 2 q range 3.35 deg and sample holder; amorphous glass equiangular 9200/2G, 0.2 mm deep. The sample was pressed with a glass plate.
Formulation 1, t=0 and 1 Month 50° C.

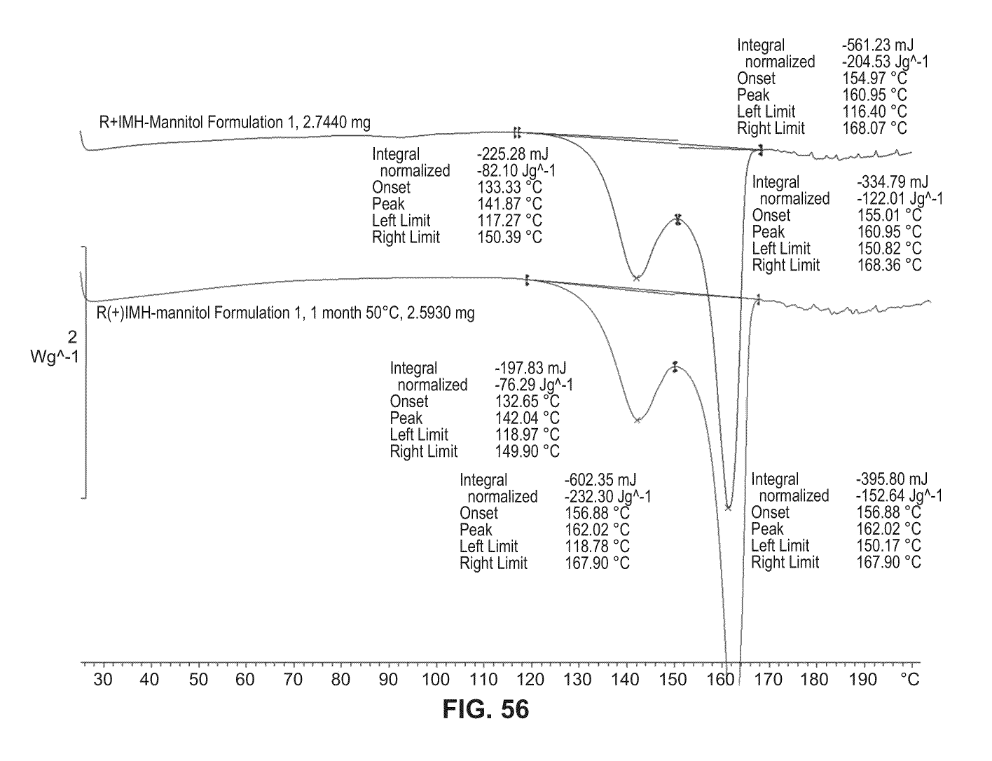

In FIG. 56 the DSC heating curves for Formulation 1 at t=0 and after 1 month at 50° C. are reported. The eutectic melting was visible at about 132-133° C. and the mannitol excess was visible at about 156° C.

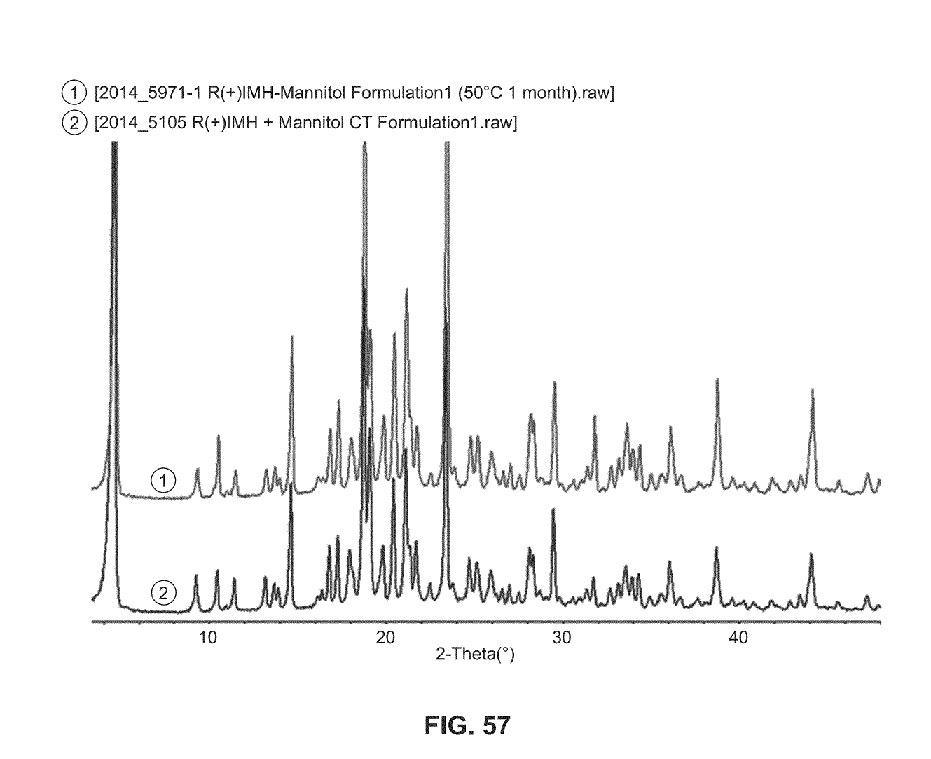

In FIG. 57 the XRPD patterns for the samples analyzed are reported. The diffraction peaks are related to the crystalline (R)-isometheptene and β-mannitol. No other peaks due to polymorphs are detected. All the samples confirm the eutectic formation with β-mannitol.

Formulation 2, t=0 and 1 Month 50° C.

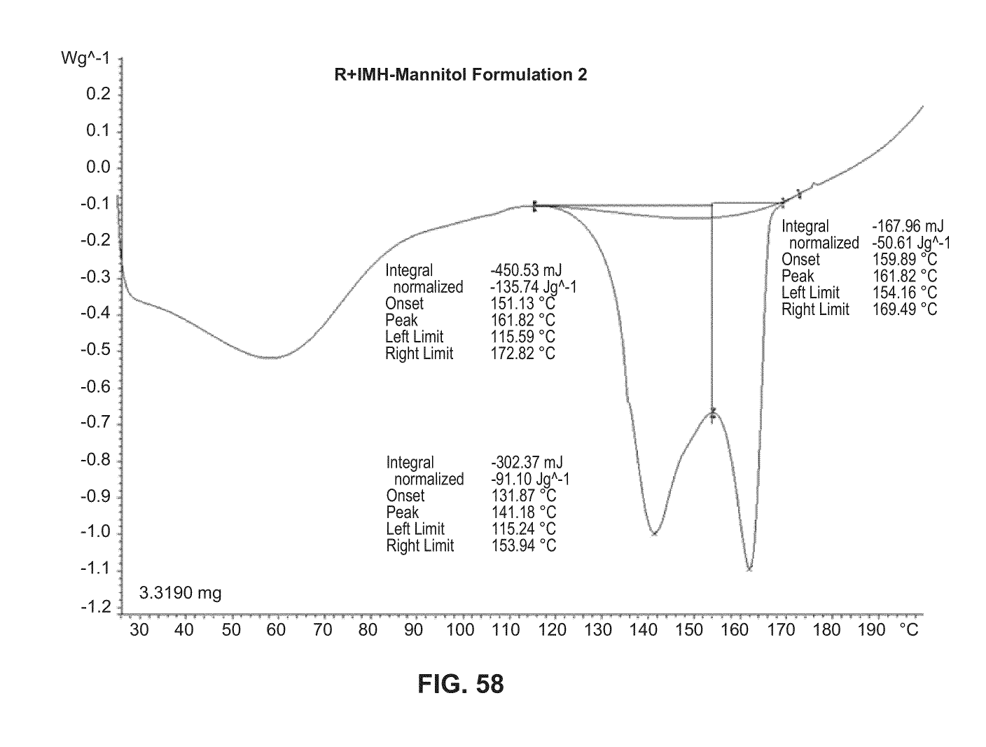

In FIG. 58 the DSC heating curves for Formulation 2 at t=0 and after 1 month at 50° C. are reported. The eutectic melting was visible between 115-173° C. (onset at 131.9° C.). The mannitol excess was visible at about 159° C.

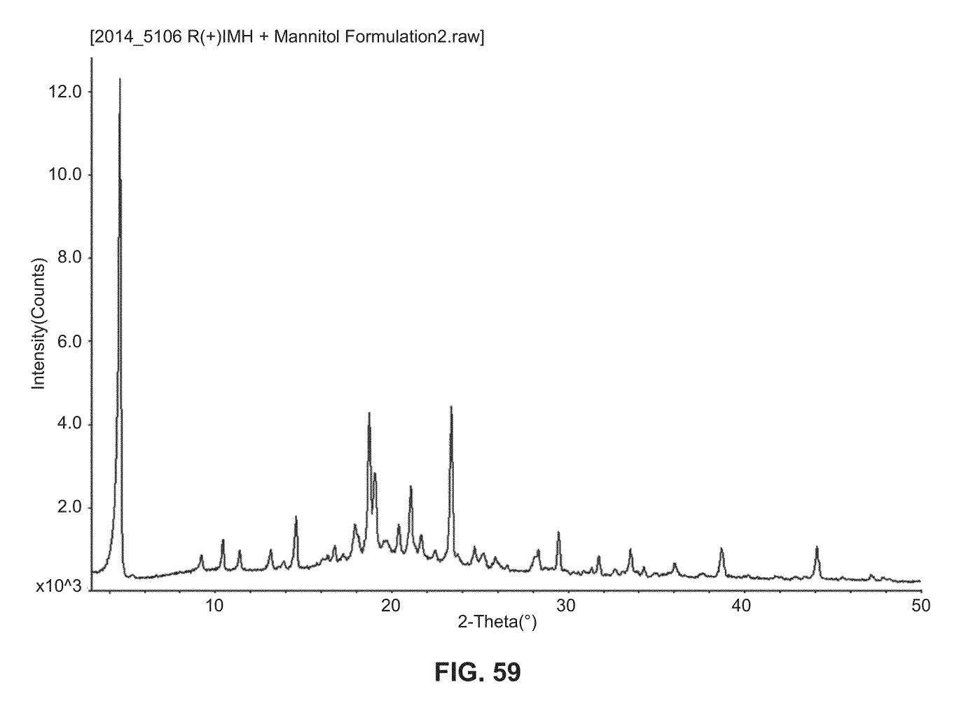

In FIG. 59 the XRPD patterns for the samples analyzed are reported. The diffraction peaks are related to the crystalline (R)-isometheptene and β-mannitol. No other peaks due to polymorphs are detected. All the samples confirm the eutectic formation with β-mannitol.

Both tablet formulations showed the presence of the eutectic with mannitol and excess mannitol at t=0 and after 50° C. for 1 month. No substantial modifications occurred to the eutectic in either formulation during the stress test for one month at 50° C.

### Example 9

Mice were given an intraplantar injection of 5% formalin (25 μL) into one posterior hindpaw. This treatment induced paw licking in control animals. Mice were briefly observed at one minute intervals between 15 and 50 minutes after the injection of formalin and the number of occasions that the mice were observed licking the injected paw was recorded. Overall, the method to detect analgesic/anti-inflammatory activity followed that described by Wheeler-Aceto, et al. (Psychopharmacology, 104. 35-44, 1991).

10 mice were studied per group. The test was performed partially blind.

The test substances selected from (R)-isometheptene mucate, and (S)-isometheptene mucate were evaluated at three doses, administered p.o. 15 minutes before the test i.e. immediately before formalin, and compared with a vehicle control group.

Morphine (32 mg/kg p.o.), 60 minutes before the test i.e. 45 minutes before formalin, was used as a reference substance.

The experiment included eight groups. Because of the number of animals, the experiment was divided into two sub-experiments (n=5 mice/group/day).

Inter-group comparison was performed for the test substance using a Ktuskaii-Waflls test, followed by Mann-Whitney U tests in case of significant group effect. For the reference substance, the treated group was compared with vehicle control using Mann-Whitney U test.

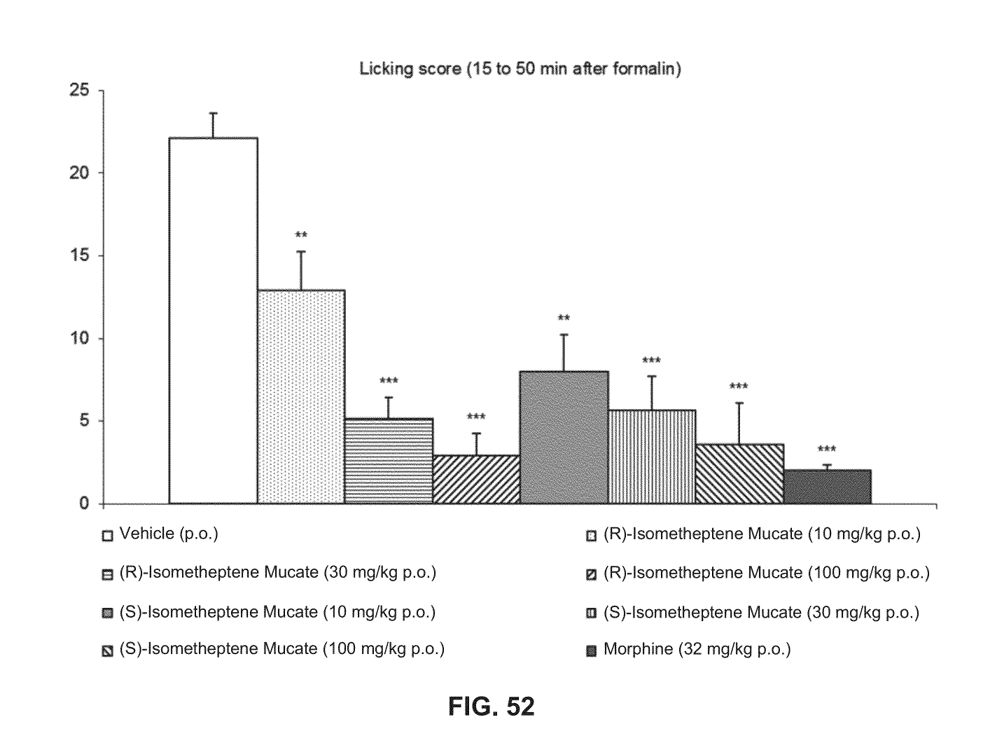

Data for (R)-isometheptene mucate, and (S)-isometheptene mucate are shown in FIG. 52. Both compounds led to a reduction in licking score compared to mice injected only with vehicle, demonstrating an analgesic effect with these compounds.

### Example 10

Mice were placed onto a hot metal plate maintained at 54° C. surrounded by a Plexiglas cylinder (height: 13 cm; diameter: 19 cm). The latency to the first foot-lick was measured (maximum: 30 seconds). Overall, the method of detecting analgesic activity followed that described by Eddy and Leimbach (J. Pharmacol. Exp. Ther., 107, 385-393, 1953).

10 mice were studied per group. The test was performed partially blind.

The test substances selected from (R)-isometheptene mucate, and (S)-isometheptene mucate were evaluated at three doses (10, 30 and 100 mg/kg), administered p.o. 15 minutes before the test, and compared with a vehicle control group.

Morphine (32 mg/kg p.o.) administered 60 minutes before the test, was used as reference substance. The experiment included 8 groups.

Data with the test substance were analyzed by comparing treated groups with vehicle control using ANOVA followed by post-hoc Dunnett's tests. Data with the reference substance were analyzed using unpaired Student's t tests.

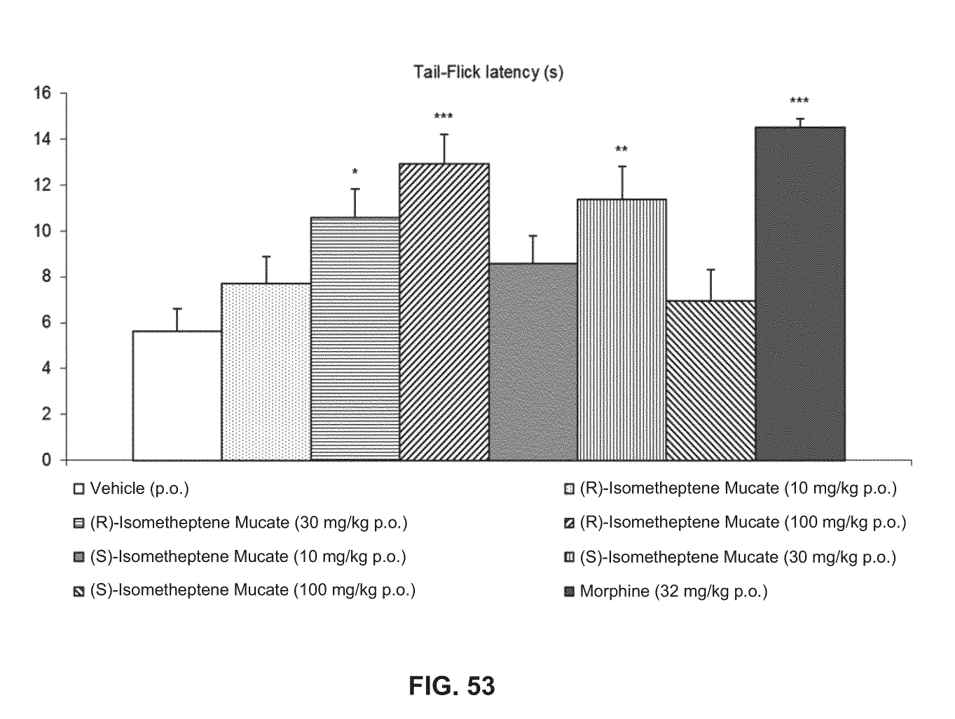

Data for compound (R)-isometheptene mucate, and (S)-isometheptene mucate, are shown in FIG. 53. Both compounds led to longer foot-licking latency times compared to mice injected only with vehicle, demonstrating an analgesic effect with these compounds.

### Example 11

A method for detecting analgesic activity followed that described by D'Amour and Smith (J. Pharmacol. Exp. Ther., 1, 74-79, 1941). The mouse's tail was heated by means of a thermal light source (20 volts). The latency before the animal withdraws its tail was measured (maximum: 15 seconds).

Ten mice were studied per group. The test was performed partially blind.

The test substances selected from (R)-isometheptene mucate, and (S)-isometheptene mucate were evaluated at three doses (10, 30, and 100 mg), administered p.o. 15 minutes before the test, and compared with a vehicle control group.

Morphine (32 mg/kg p.o.) 60 minutes before the test, was used as reference substance. The experiment included eight groups.

Data with the test substance were analyzed by comparing treated groups with vehicle control using ANOVA followed by post-hoc Dunnett's tests. Data with the reference substance were analyzed using unpaired Student's t tests.

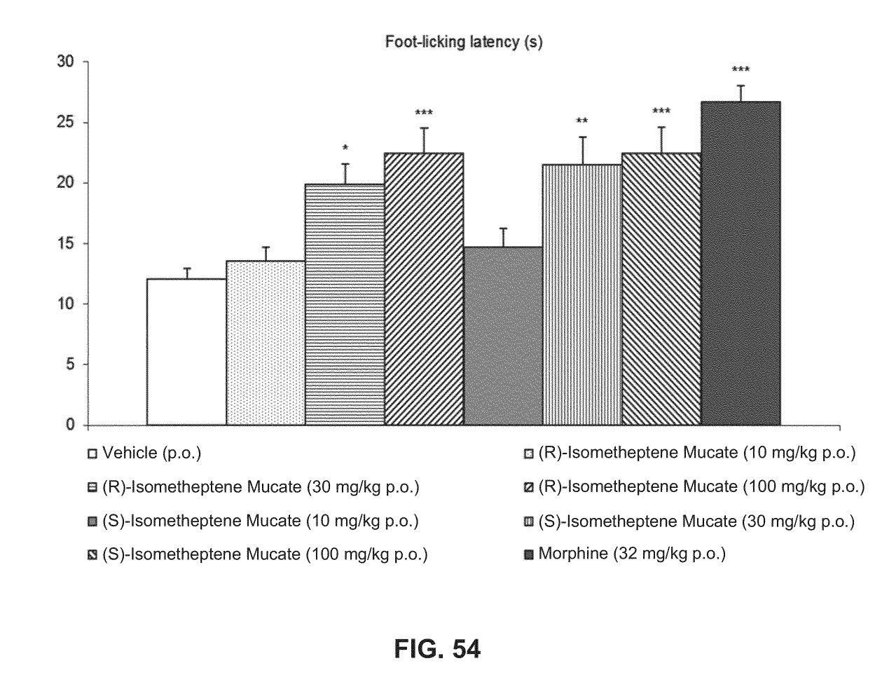

Data for (R)-isometheptene mucate and (5)-isometheptene mucate, are shown in FIG. 54. Both compounds led to longer tail flick latency times compared to mice injected only with vehicle, demonstrating an analgesic effect with these compounds. (R)-isometheptene mucate at 100 mg/kg p.o. showed activity similar to that of morphine at 32 mg/kg.

### Example 12

Spontaneous trigeminal allodynia (STA) rats are rats with the inherited trait of spontaneously changing trigeminal von Frey thresholds. Protocols for testing these rats were adapted from Oshinsky, M. L., et al., Spontaneous Trigeminal Allodynia in Rats: A Model of Primary Headache, 2012, 52: 1336-1349. Oshinsky et al. describes these rats as a novel model of spontaneous headache that can be used as a model of primary headache.

STA rats and litter mates without the trait were injected with compounds selected from (R)-isometheptene mucate and (5)-isometheptene mucate on days when their thresholds in spontaneous allodynia rats were 4 g or below for STA rats. Testing days for each of the compounds were separated by at least one week. Tactile sensory thresholds were recorded prior to and following injections at 0.5 hours, 1.5 hours, 2.5 hours, 3.5 hours, and 24 hours.

### Tactile Sensory Testing

Rats were trained and acclimated to a plastic tube restraint and entered uncoaxed. This restrainer allowed the rats to undergo sensory testing.

Periorbital, hind-paw, and jaw-pressure thresholds were determined by applying von Frey monofilaments (Stoelting Co., Wood Dale, Ill., USA). Each monofilament was identified by manufacturer-assigned force values (26, 15, 10, 8, 6, 4, 2, 1.4, 1, 0.6, 0.4, 0.07 g). For trigeminal testing, the filaments were tested on both the left and right sides of the face, over the rostral portion of the eye for periorbital testing, and on the skin over the masseter muscle for jaw testing. The vibrissae were not touched during testing. For the hind-paw testing, the filaments were applied to the mid-plantar region of the left and right hind paws, avoiding the less sensitive foot pads. For the hind-paw testing, the maximum value tested was 26 g; the rats that did not respond to this stimulus were assigned this value. Left and right threshold data were recorded separately. The von Frey stimuli were presented in sequential order, either ascending or descending, as necessary, to determine the threshold of response. After a positive response, a weaker stimulus was presented, and after a negative response, a stronger stimulus is presented.

Results were presented either as the threshold in grams±standard error of the mean (SEM), or as a percent change from baseline on the side that has the lowest value. The threshold was defined as a positive response to 2 of 3, or in some cases 3 of 5 trials of a single von Frey monofilament. The value of the von Frey filament that elicited head withdrawal in 2 of 3 repetitions of the stimulus was designated as that day's threshold. Several behaviors were considered a positive head-withdrawal response, including when the rat vigorously stroked its face with the ipsilateral forepaw and quickly recoiled its head away from the stimulus or vocalized. For the periorbital von Frey testing, rats that did not respond to the 10-g stimulus were assigned 10 g as their threshold.

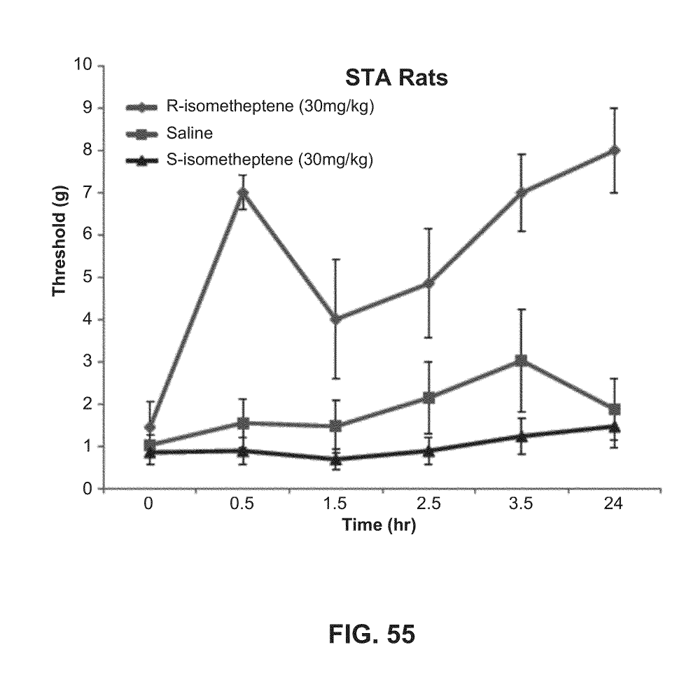

Data for (R)-isometheptene mucate and (5)-isometheptene mucate are shown in FIG. 55. STA rats treated with (R)-isometheptene mucate showed a dramatic increase in threshold values versus STA rats treated with (S)-isometheptene mucate or the control rats, demonstrating the analgesic effect of (R)-isometheptene mucate.

What is claimed is:

1. A pharmaceutical composition comprising a eutectic of mannitol and (R)-isometheptene mucate.

2. The pharmaceutical composition of claim 1, wherein the mannitol is (3-mannitol.

3. The pharmaceutical composition of claim 2, wherein the eutectic melts at 134±4° C.

4. The pharmaceutical composition of claim 1, wherein the mannitol is 6-mannitol.

5. The pharmaceutical composition of claim 4, wherein the eutectic melts at 120±4° C.

6. The pharmaceutical composition of claim 1, comprising 60%-90% (R)-isometheptene mucate and 40%-10% mannitol by weight.

7. The pharmaceutical composition of claim 6, comprising amounts of (R)-isometheptene mucate and mannitol selected from: 60%±2% (R)-isometheptene mucate and 40%±2% mannitol by weight, 65%±2% (R)-isometheptene mucate and 35%±2% mannitol by weight, 70%±2% (R)-isometheptene mucate and 30%±2% mannitol by weight, 75%±2% (R)-isometheptene mucate and 25%±2% mannitol by weight, 80%±2% (R)-isometheptene mucate and 20%±2% mannitol by weight, 85%±2% (R)-isometheptene mucate and 15%±2% mannitol by weight, and 90%±2% (R)-isometheptene mucate and 10%±2% mannitol by weight.

8. The pharmaceutical composition of claim 7, comprising 75%±2% (R)-isometheptene mucate and 25%±2% mannitol by weight.

9. The pharmaceutical composition of claim 1, further comprising one or more excipients.

10. A method of treating a condition selected from pain, tension-type headache (TTH), allodynia, and fibromyalgia in a patient in need thereof, comprising administering to said patient a therapeutically effective amount of the pharmaceutical composition according to claim 1.

11. The method of claim 10, wherein the condition is pain.

12. The method of claim 10, wherein the condition is tension-type headache (TTH).

13. The method of claim 10, wherein the condition is allodynia.

14. The method of claim 10, wherein the condition is fibromyalgia.

15. The method of claim 10, wherein the pharmaceutical composition is administered with one or more substances selected from the group consisting of acetaminophen, non-steroidal anti-inflammatory drugs (NSAIDs), ibuprofen, naprosyn, cyclooxygenase-2 inhibitors, aspirin, caffeine, dichloralphenazone, triptans, antidepressants, serotonin-norepinephrine reuptake inhibitors (SNRIs), and gabapentinoids.

16. The method of claim 10, wherein the pharmaceutical composition is administered with one or more opiates.
